(19) United States
(12) Patent Application Publication
(10) Pub. No.: US 2025/0339712 A1
(43) Pub. Date: Nov. 6, 2025

(54) ADAPTIVE CORRELATION FILTER FOR RADIOTHERAPY

(71) Applicant: RefleXion Medical, Inc., Hayward, CA (US)

(72) Inventors: Shiyu XU, San Ramon, CA (US); Jayakrishnan JANARDHANAN, Union City, CA (US); Peter Demetri OLCOTT, Los Gatos, CA (US); George Andrew ZDASIUK, Portola Valley, CA (US)

(21) Appl. No.: 19/208,034

(22) Filed: May 14, 2025

Related U.S. Application Data
(63) Continuation of application No. PCT/US2023/079652, filed on Nov. 14, 2023.
(60) Provisional application No. 63/383,851, filed on Nov. 15, 2022.

(51) Int. Cl. A61N 5/10; G06T 7/13; G06T 7/32; G06T 7/33; G06T 7/73; G16H 20/40; G16H 30/40

(52) U.S. Cl. CPC A61N 5/1039; A61N 5/1049; A61N 5/1067; G06T 7/13; G06T 7/32; G06T 7/337; G06T 7/74; G16H 20/40; G16H 30/40; A61N 2005/1052; A61N 5/1075; G06T 2207/10081; G06T 2207/10104; G06T 2207/20081; G06T 2207/30096

(57) ABSTRACT

Disclosed herein are systems and methods of applying a tracking filter, such as an adaptive correlation filter (ACF), to imaging data acquired during a radiotherapy session and using the filtered image to guide the delivery of radiation. The filtered image may provide information about the real-time location of the target anchor and the radiotherapy system may then calculate a radiation fluence using the target anchor location information. The methods described herein may be used with image-guided radiotherapy (such as IMRT/SBRT/SRS), as well as biology-guided radiotherapy (BgRT), which is a type of radiotherapy that converts biologically-related imaging data acquired on the day of treatment into radiation fluences for delivery.

### CROSS-REFERENCE TO RELATED APPLICATIONS

This application is a continuation of International Patent Application No. PCT/US2023/079652, filed Nov. 14, 2023, which claims priority to U.S. Provisional Patent Application No. 63/383,851 filed Nov. 15, 2022, which is hereby incorporated by reference in its entirety.

### BACKGROUND

Various radiotherapy treatments, such as stereotactic body radiation therapy (SBRT), intensity modulated radiation therapy (IMRT), or biology-guided radiotherapy (BgRT) use imaging data (e.g., CT images) to position a patient during a radiotherapy session. BgRT uses imaging data to guide radiotherapy delivery. In various cases, a location of a tumor requiring treatment may need to be determined based on the available imaging data. Accordingly, systems and methods for making such determinations are desirable.

### SUMMARY

Disclosed herein are systems and methods for determining a radiation fluence for delivery such that radiation is delivered to a real-time location of a tumor. In one variation, a method may include acquiring imaging data of a target region, generating a filtered image by applying a tracking filter (such as an adaptive correlation filter) to the imaging data, determining a target anchor location from the filtered image, and calculating a radiation fluence to be delivered to the target region using the target anchor location.

In some variations of the method, calculating the radiation fluence includes applying a firing filter to an image of the target anchor location, where the firing filter is calculated based on previously-acquired imaging data of the target region.

In some variations of the method, calculating the radiation fluence includes shifting a planned fluence according to the target anchor location.

In some variations, the method further includes delivering the calculated radiation fluence to the target region with a therapeutic radiation source. While the therapeutic radiation source may be used for patient treatment, in some variations, the therapeutic radiation source may be used in a non-therapeutic session, for example, for quality assurance and/or testing purposes, where a phantom and/or radiation measurement devices are irradiated, and not a patient.

In some variations, the method further includes updating the adaptive correlation filter with additional imaging data.

In some variations, the method further includes acquiring the additional imaging data and updating the adaptive correlation filter before generating the filtered image.

In some variations of the method, additional imaging data is simulated imaging data.

In some variations of the method, the acquired imaging data is obtained using a positron emission tomography (PET) imaging system, for example, a diagnostic PET imaging system.

In some variations, the method further includes determining a location of a planning contour using the acquired imaging data. In some variations, determining the target anchor location includes locating the target anchor relative to the location of the planning contour.

In some variations of the method, the planning contour may be the boundary of a planning target volume (PTV), and the target anchor location is a location of a centroid of the PTV.

In some variations of the method, the planning contour may be the boundary of a planning target volume (PTV), and the target anchor location may be a point (or region) on or within the boundary of the PTV.

In some variations of the method, a planning contour may be the boundary of an organ-at-risk (OAR), and the target anchor location may be a location on or within the boundary of the OAR. In some variations, the method further includes delivering the calculated radiation fluence, acquiring additional imaging data, updating the target anchor location, calculating a second radiation fluence using the updated target anchor location, and delivering the second radiation fluence. While the radiation may be delivered for patient treatment, in some variations, the radiation may be delivered in a non-therapeutic session, for example, for quality assurance and/or testing purposes where a phantom and/or radiation measurement devices are irradiated, and not a patient.

In some variations of the method, the acquired imaging data may be obtained using a diagnostic positron emission tomography (PET) imaging system, and/or the acquired additional image data may be obtained using a biology-guided radiotherapy (BgRT) PET imaging system.

In some variations of the method, the planned fluence includes a segmented multi-leaf collimator (MLC) leaf pattern and calculating the radiation fluence includes shifting the segmented MLC leaf pattern according to the target anchor position.

In some variations of the method, applying the adaptive correlation filter to the imaging data includes cross-correlating the adaptive correlation filter with the imaging data.

In some variations of the method, applying the firing filter to the target anchor location includes convolving an image of the target anchor location with the firing filter.

In some variations of the method, the imaging data includes a set of PET images of the target region obtained over a plurality of patient platform positions during a PET pre-scan. Further, in some variations, the method may include, prior to generating the filtered image, aligning one or more images from the set of PET images with at least one CT image using a planned anchor location and/or contour of the target region, updating the adaptive correlation filter based on the aligned set of PET images, and acquiring additional imaging data.

In some variations, the method further includes, after calculating the radiation fluence, delivering the calculated radiation fluence to the target region while acquiring additional PET imaging data, updating the anchor location based on the further additional PET imaging data, and updating the adaptive correlation filter based on the additional PET imaging data. While the radiation may be delivered for patient treatment, in some variations, the radiation may be delivered in a non-therapeutic session, for example, for quality assurance and/or testing purposes where a phantom and/or radiation measurement devices are irradiated, and not a patient.

In some variations, the method further includes evaluating a confidence metric of the filtered image to determine whether the confidence metric exceeds a threshold confidence value.

In some variations, the method further includes delivering the calculated radiation fluence while acquiring additional PET imaging data at a position of a patient platform and updating the adaptive correlation filter based on the additional PET imaging data. While the radiation may be delivered for patient treatment, in some variations, the radiation may be delivered in a non-therapeutic session, for example, for quality assurance and/or testing purposes where a phantom and/or radiation measurement devices are irradiated, and not a patient.

In some variations of the method, the patient platform may be placed into a plurality of positions. Further, in some variations the method includes repeating at least once: moving the patient platform to a next position, such that the next position becomes the position at which the calculated radiation fluence is being delivered, and at which the adaptive correlation filter is being updated. While the radiation may be delivered for patient treatment, in some variations, the radiation may be delivered in a non-therapeutic session, for example, for quality assurance and/or testing purposes where a phantom and/or radiation measurement devices are irradiated, and not a patient.

In some variations, the method further includes repeating steps of delivering the calculated radiation fluence to the target region while acquiring additional PET imaging data, updating the anchor location based on the further additional PET imaging data, and updating the adaptive correlation filter based on the additional PET imaging data, if the prescribed radiation is not delivered.

In some variations, the method further includes generating a graphical representation of the acquired PET imaging data.

In some variations of the method, the calculated radiation fluence may be derived from the acquired PET imaging data that has been filtered by the adaptive correlation filter.

In some variations, the method further includes delivering the calculated radiation fluence while acquiring additional PET imaging data during a patient platform shuttle pass and updating the adaptive correlation filter based on the additional PET imaging data. While the radiation may be delivered for patient treatment, in some variations, the radiation may be delivered in a non-therapeutic session, for example, for quality assurance and/or testing purposes where a phantom and/or radiation measurement devices are irradiated, and not a patient.

In some variations of the method, a plurality of couch shuttle passes may be performed, and where steps of the method, such as, delivering the calculated radiation fluence while acquiring additional PET imaging data during a patient platform shuttle pass and updating the adaptive correlation filter based on the additional PET imaging data, when it is determined that prescribed radiation for a treatment session has not been delivered, are performed for each pass of the plurality of couch shuttle passes. While the radiation may be delivered for patient treatment, in some variations, the radiation may be delivered in a non-therapeutic session, for example, for quality assurance and/or testing purposes where a phantom and/or radiation measurement devices are irradiated, and not a patient.

In other variations of the method, the target anchor location may be the location of a first target anchor, the adaptive correlation filter is a first adaptive correlation filter, and the filtered image is a first filtered image, and the method may further comprise determining a target region rotation or tilt using the location of the first target anchor, a location of a second target anchor and a location of a third target anchor, and where calculating the radiation fluence to be delivered may comprise applying the target region rotation or tilt to a planned radiation fluence. Determining the target region rotation or tilt may comprise generating a second filtered image by applying a second adaptive correlation filter to the imaging data, determining the second target anchor location from the second filtered image, generating a third filtered image by applying a third adaptive correlation filter to the imaging data, determining the third target anchor location from the third filtered image, defining a delivery orientation plane using the first, second and third target anchor locations, and determining an angular rotation or tilt of the target region by comparing the delivery orientation plane with a planning orientation plane of the target region. The acquired imaging data may be 3-D imaging data, and the first target anchor location, the second target anchor location, and the third target anchor location may be defined using 3-D coordinates.

Also disclosed herein is a radiation therapy system. In one variation, the radiation therapy system includes a rotatable gantry, a therapeutic radiation source mounted on the gantry, one or more imaging sensors mounted on the gantry to acquire imaging data of a target region, and a controller in communication with the gantry, the therapeutic radiation source, and the one or more imaging sensors, the controller configured to apply an adaptive correlation filter to the acquired imaging data to generate a cross-correlation image, determine a target anchor location from the cross-correlation image, and to calculate a radiation fluence to be delivered to the target region by convolving an image of the target anchor location with a firing filter.

In some variations of the system, the firing filter may be calculated based on previously-acquired imaging data of the target region, for example, previously-acquired imaging data of the target anchor location, which may include a Gaussian or delta function centered over the target anchor location.

In some variations of the system, the controller may be further configured to deliver the calculated radiation fluence to the target region with a therapeutic radiation source.

In some variations of the system, the controller may be further configured to update the adaptive correlation filter with additional imaging data.

In some variations of the system, the controller may be further configured to acquire the additional imaging data prior to updating the adaptive correlation filter.

In some variations of the system, the additional imaging data may be simulation imaging data.

In some variations of the system, the acquired imaging data may be obtained using a positron emission tomography (PET) imaging system, for example, a diagnostic PET imaging system.

In some variations of the system, the controller may be further configured to determine a location of a planning contour using the acquired imaging data. In some variations, determining the target anchor location may comprise locating the target anchor relative to the location of the planning contour.

In some variations of the system, the planning contour may be the boundary of a planned target volume (PTV), and the target anchor location is a location of a centroid of the PTV. In some variations, the planning contour may be the boundary of an organ-at-risk (OAR), and the target anchor location may be a point on or within the boundary of the OAR.

In some variations of the system, the planning contour may be the boundary of a planned target volume (PTV), and the target anchor location may be a point on or within the boundary of the PTV.

In some variations of the system, the controller is further configured to deliver the calculated radiation fluence, acquire additional imaging data, update the target anchor location, calculate a second radiation fluence using the updated target anchor location, and deliver the second radiation fluence.

In some variations of the system, the acquired imaging data may be obtained using a diagnostic positron emission tomography (PET) imaging system, and/or the acquired additional image data may be obtained using a biology-guided radiotherapy (BgRT) PET imaging system.

In some variations of the system, applying the adaptive correlation filter to the imaging data may comprise cross-correlating the adaptive correlation filter with the imaging data.

In some variations of the system, the imaging data includes a set of PET images of the target region obtained over a plurality of patient platform positions during a PET pre-scan. In some variations, the controller may be further configured to align one or more images from the set of PET images with at least one CT image using a planned target anchor location and/or contour of the target region, update the adaptive correlation filter based on the aligned set of PET images, and acquire additional imaging data.

In some variations of the system, the controller may be further configured to, after calculating the radiation fluence, deliver the calculated radiation fluence to the target region while acquiring additional PET imaging data, update the target anchor location based on the further additional PET imaging data, and update the adaptive correlation filter based on the additional PET imaging data.

In some variations of the system, applying the adaptive correlation filter to the imaging data includes cross-correlating the adaptive correlation filter with the imaging data resulted in a filtered image. Further, in some variations, the controller is further configured to evaluate a confidence metric of the filtered image to determine whether the confidence metric exceeds a threshold confidence value.

In some variations of the system, the controller may be further configured to deliver the calculated radiation fluence while acquiring additional PET imaging data at a position of a patient platform and update the adaptive correlation filter based on the additional PET imaging data, e.g., when it is determined that prescribed radiation for a treatment session has not been delivered.

In some variations of the system, the patient platform may be placed into a plurality of positions. Further, in some variations, the controller may be configured to repeat at least once: move the patient platform to a next position, such that the next position becomes the position at which the calculated radiation fluence is being delivered, and at which the adaptive correlation filter is being updated.

In some variations, the system may include a display. Further, in some variations, the controller may be configured to generate a signal corresponding to a graphical representation of the acquired PET imaging data and output the signal to the display.

In some variations of the system, the calculated radiation fluence may be derived from the acquired PET imaging data that has been filtered by the adaptive correlation filter.

In some variations, the target anchor location may be the location of a first target anchor location, the adaptive correlation filter may be a first adaptive correlation filter, and the cross-correlation image may be a first cross-correlation image, and the controller may be configured to determine a target region rotation or tilt using the location of the first target anchor, a location of a second target anchor and a location of a third target anchor. Calculating the radiation fluence to be delivered may comprise applying the target region rotation or tilt to a planned radiation fluence. In some variations, determining the target region rotation or tilt may comprise generating a second cross-correlation image by applying a second adaptive correlation filter to the imaging data, determining the second target anchor location from the second cross-correlation image, generating a third cross-correlation image by applying a third adaptive correlation filter to the imaging data, determining the third target anchor location from the third cross-correlation image, defining a delivery orientation plane using the first, second and third target anchor locations, and determining an angular rotation or tilt of the target region by comparing the delivery orientation plane with a planning orientation plane of the target region. The acquired imaging data may be 3-D imaging data, and the first target anchor location, the second target anchor location, and the third target anchor location may be defined using 3-D coordinates.

Also disclosed herein is a method of generating an adaptive correlation filter. The method may include acquiring CT imaging data and PET imaging data of a target region, selecting a target anchor location based on a contour of the target region defined in the CT imaging data, aligning PET imaging data with the contour, defining an image of an anchor function, and generating an adaptive correlation filter using the set of PET images and the image of the anchor function. The anchor function may be centered on the target anchor location.

In some variations of the method, generating an adaptive correlation filter may include selecting the adaptive correlation filter that minimizes a measure function based on a difference between results of applying the adaptive correlation filter to the set of PET images and the image anchor function.

In some variations of the method, the anchor function may be a delta function centered on the target anchor location. In some variations, the anchor function may comprise a plurality of delta functions (e.g., two or more) around or centered on the target anchor location.

Further, disclosed herein is another variation of a method of generating an adaptive correlation filter. The method includes acquiring imaging data of a target region over a plurality of patient platform positions, selecting a planned target anchor location based on a contour of the target region in the acquired imaging data, aligning the acquired imaging data with the contour, defining an image of an anchor function centered on the planned target anchor location, and generating an adaptive correlation filter using the acquired imaging data and the image of the anchor function.

In some variations of the method, generating an adaptive correlation filter may include selecting the adaptive correlation filter that minimizes a measure function based on a difference between results of applying the adaptive correlation filter to the acquired imaging data and the image of the anchor function.

In some variations, the method includes updating the adaptive correlation filter. In some variations, updating the adaptive correlation filter may include obtaining updated imaging data, aligning the updated imaging data with respect to the planned target anchor position and/or the contour, and updating the adaptive correlation filter using the aligned updated imaging data and the image of the anchor function.

In some variations of the method, the anchor function may comprise a Gaussian function centered around or on the target anchor location.

In some variations of the method, the imaging data may be CT imaging data.

In some variations of the method, the anchor function may be a delta function centered on the planned target anchor location. In some variations, the anchor function may comprise a plurality of delta functions (e.g., two or more) around or centered on the target anchor location. In some variations, the anchor function may comprise a Gaussian function centered around or on the target anchor location.

Further, disclosed herein is a system for generating an adaptive correlation filter using CT imaging data and PET imaging data. The system includes a memory for storing imaging data and instructions, and a processor configured to execute the instructions to perform operations. The operations may include receiving an input from a user to select a planned anchor location based on a contour of the target region in the CT imaging data, aligning the PET imaging data with respect to the contour of the target region, defining an image of an anchor function centered on the planned anchor location, and generating an adaptive correlation filter using the PET imaging data and the image of the anchor function.

In some variations, the system includes a display for displaying a graphical user interface for interacting with the processor.

In some variations of the system, the processor is further configured to generate a graphical user interface (GUI) that includes the CT imaging data and/or PET imaging data and to output the GUI to the display.

In some variations of the system, the GUI may include a graphical representation of the planned target anchor location and/or image of the anchor function depicted simultaneously with the contour of the target region.

Also disclosed herein is a method for calculating a radiation fluence for delivery using one or more target anchors for a target region. In one variation, the method may comprise defining a plurality of target anchors for a contour of a target region on a planning image, wherein the target anchors define a planning orientation volume, generating an adaptive correlation filter corresponding to each of the plurality of target anchors and the target region contour, acquiring imaging data of the target region during radiation delivery session, applying the adaptive correlation filters to the acquired imaging data to generate a cross-correlation image corresponding to each of the plurality of target anchors, determining a target anchor locations in each of the cross-correlation images, defining a delivery orientation volume using the identified target anchor locations, determining an angular rotation or tilt of the target region by comparing the delivery orientation volume with the planning orientation volume, and calculating a delivery radiation fluence by applying the angular rotation or tilt to a planned radiation fluence map. The planning orientation volume may be a planning orientation plane and the delivery orientation volume may be a delivery orientation plane. Optionally, the method may further comprise training each of the generated adaptive correlation filters with additional imaging data. The plurality of target anchors may comprise one or more of distinctive features or landmarks relative to the contour of the target region. The distinctive features or landmarks may comprise locations that are geometrically-derived from the contour of the target region. For example, the distinctive features or landmarks may comprise locations of anatomical structures. The plurality of target anchors may comprise points on projections of the target region contour on orthogonal planes in a 3-D coordinate system. The method may further comprise segmenting the delivery radiation fluence into a set of radiotherapy machine instructions. The radiotherapy machine instructions may comprise multi-leaf collimator configurations and therapeutic radiation source pulse characteristics. Optionally, some methods may further comprise delivering radiation beamlets according to the radiotherapy machine instructions.

The methods described herein may also be used for patient localization. In some variation, a method for localizing a patient for radiotherapy may comprise acquiring PET imaging data of a patient in a treatment position on a patient platform, generating a cross-correlation image by applying an adaptive correlation filter to the acquired PET imaging data, determining a location of a target anchor using the cross-correlation image, comparing the location of the target anchor with its planned location to generate a set of shift vectors, and moving the patient platform according to the shift vectors. Some methods may further comprise determining a location of a target region contour based on the determined location of the target anchor, and comparing the location of the target region contour with its planned location to generate the shift vectors. The adaptive correlation filter may be generated during treatment planning based on a planning image of the planned target anchor location and its corresponding target region contour. Optionally, the target region contour may be a contour of an organ-at-risk (OAR). In some variations, the above steps may be applied to a first target region having a first target anchor location, and the method may further comprise generating a second cross-correlation image of a second target region by applying a second adaptive correlation filter to the acquired PET imaging data, determining a location of a second target anchor using the second cross-correlation image, comparing the location of the second target anchor with its planned location to generate a second set of shift vectors, and applying the second set of shift vectors to a planned radiation fluence map for the second target region.

Alternatively, or additionally, a method for localizing a patient for radiotherapy may comprise acquiring PET imaging data of a patient in a treatment position on a patient platform, generating a cross-correlation image by applying an adaptive correlation filter to the acquired PET imaging data, determining a location of a target anchor using the cross-correlation image, comparing the location of the target anchor with its planned location to generate a set of shift vectors, and applying the set of shift vectors to a planned radiation fluence map for the target region.

Described herein are methods for updating an adaptive correlation filter. One variation of a method may comprise acquiring PET imaging data of a patient in a treatment position on a patient platform, shifting the acquired PET imaging data to align with a planned contour of the target region and/or a planned anchor location, and updating an adaptive correlation filter based on the shifted PET imaging data and the planned anchor location. The method may further comprise determining whether the shift of the acquired PET imaging data is outside an acceptable range of shifts, and if the shift is outside the acceptable range of shifts, the method may further comprise generating a notification to an operator. Optionally, the method may comprise generating a graphical user interface that may comprise the planned location of the target anchor, the planned location of the target region contour, and the PET imaging data. The graphical user interface may optionally comprise control indicia that is configured to allow an operator to manually shift the PET imaging data to align with the planned location of the target region contour. Updating the adaptive correlation filter may be based on the manually shifted PET imaging data and the planned location of the target anchor.

The methods described herein may be used for quality assurance (QA) of a BgRT treatment plan. A QA method is non-therapeutic and does not irradiate or treat a patient. One variation of a QA method of a BgRT treatment plan may comprise placing a phantom having a positron-emitting point source and radiation measurement devices on a platform of a biology-guided radiotherapy (BgRT) system, generating an image of a target anchor location by measuring positron emissions from the point source and applying an adaptive correlation filter to the positron emission data to identify a location of the target anchor, delivering radiation according to a BgRT treatment plan by convolving an image of the target anchor location with firing filters to generate a delivery fluence map, and emitting radiation according to the delivery fluence map, measuring the emitted radiation using the radiation measurement devices of the phantom, calculating the delivered dose distribution based on the radiation measurements, and evaluating the quality of the BgRT treatment plan by comparing the delivered dose distribution with a planned dose distribution. The planned dose distribution may comprise a radiation dose to the phantom based on a CT image of the phantom and a planned fluence map of the BgRT treatment plan. Measuring the emitted radiation may comprise using a megavoltage (MV) detector of the BgRT radiotherapy system. The positron-emitting point source may have a radioactivity of about 1 μCi to 300 μCi. In some variations, the positron-emitting point source may comprise one or more of the following isotopes: 22-Na, 68-Ge, 18-F, 68-Ga, 11-C, 64-Cu, 82-Rb, 13-N, and/or 89-Zr.

The foregoing general description and the following detailed description are exemplary and explanatory only and are not restrictive of the claims. While the examples of the methods and systems described herein may be in the context of delivering radiation for therapeutic purposes, it should be understood that any and all of these methods and systems may be used for non-therapeutic purposes, such as for quality assurance and/or testing purposes where radiation is delivered to a phantom and/or radiation measurement devices. In these non-therapeutic applications, a patient is not present and not being irradiated.

### BRIEF DESCRIPTION OF THE DRAWINGS

FIG. 1A is a block diagram of one variation of a radiotherapy system.

FIG. 1B is a flowchart of one variation of a radiotherapy workflow.

FIG. 2A depicts one variation of a radiotherapy system.

FIG. 2B depicts another variation of a radiotherapy system.

FIG. 3A is one variation of a method of using an adaptive correlation filter for calculating a radiation fluence.

FIG. 3B is another variation of a method of using an adaptive correlation filter for calculating a radiation fluence.

FIGS. 3C-3E are simulation results depicting PET imaging data, PET imaging data filtered with an ACF, and an image of a target anchor location, respectively.

FIG. 4A is one variation of a method of generating an adaptive correlation filter.

FIGS. 4B-4F depict conceptual representations of the method of FIG. 4A.

FIG. 5A is a variation of a method of generating an adaptive correlation filter.

FIGS. 5B-5D depict spatial domain plots of an adaptive correlation filter (i.e., an ACF template). FIG. 5B is an axial view, FIG. 5C, is a sagittal view, and FIG. 5D is a coronal view of the adaptive correlation filter.

FIG. 6 is a variation of a method of updating an adaptive correlation filter.

FIG. 7A is one variation of a method of using an adaptive correlation filter during a BgRT treatment.

FIGS. 7B-7C is another variation of a method of using an adaptive correlation filter during a BgRT treatment.

FIG. 7D is one variation of a method for calculating a confidence metric for a filtered image.

FIGS. 7E-7M depict conceptual representations of the method shown in FIGS. 7B-7C.

FIG. 8A is one variation of a method of using an adaptive correlation filter during a BgRT treatment with a target anchor location update.

FIG. 8B is one variation of a method of updating an adaptive correlation filter during a BgRT treatment.

FIG. 8C is another variation of a method of updating an adaptive correlation filter during a BgRT treatment.

FIG. 9A is one variation of a method of calculating a radiation fluence during a BgRT treatment using a target anchor location.

FIG. 9B is another variation of a method of calculating a radiation fluence during a BgRT treatment using a target anchor location.

FIG. 10A is one variation of a method of generating an adaptive correlation filter during treatment planning and updating the adaptive correlation filter during a BgRT treatment.

FIG. 10B is another variation of a method of generating an adaptive correlation filter during treatment planning and updating the adaptive correlation filter.

FIG. 11A is one variation of a method of using an adaptive correlation filter for delivering a radiation fluence during a radiotherapy treatment (e.g., IMRT, SBRT, and/or SRS).

FIG. 11B is another variation of a method of updating and using an adaptive correlation filter for delivering a radiation fluence during a radiotherapy treatment (e.g., IMRT, SBRT, and/or SRS).

FIG. 12 depicts a flowchart representation of one variation of a method comprising the use of multiple target anchors for a single target region, calculating multiple adaptive correlation filters for the multiple target anchors, and applying the multiple adaptive correlation filters to imaging data acquired during radiation delivery.

FIG. 13 depicts a flowchart representation of one variation of a method for localizing a patient for radiation delivery.

FIG. 14A depicts a flowchart representation of one variation of a method for updating or generating an adaptive correlation filter to account for changes in the PET signal. FIGS. 14B-14C are conceptual depictions of one example of a graphical user interface that may be used to shift a target region contour calculated based on a PET signal into alignment with the planned target region contour and anchor.

FIG. 15 depicts a flowchart representation of one variation of a method for quality assurance (QA) of a patient-specific biology-guided radiotherapy (BgRT) treatment plan generated using the methods described herein.

### DETAILED DESCRIPTION

Disclosed herein are systems and methods of applying a tracking filter, such as an adaptive correlation filter (ACF), to imaging data acquired during a radiotherapy session and using the filtered image to guide the delivery of radiation. The methods described herein may be used with biology-guided radiotherapy (BgRT), which is a type of radiotherapy that converts biologically-related imaging data acquired on the day of treatment into radiation fluences for delivery. This may facilitate the delivery of radiation to the real-time location of a tumor. In some variations, the imaging data is acquired and converted into radiation fluences for delivery in real-time. This conversion from imaging data into radiation fluence uses a mapping function that designates the relationship between imaging data and radiation fluence and is configured to transform imaging data into a corresponding radiation fluence. In some variations, the mapping function comprises firing filters. The mapping function (or firing filters) is calculated for each target region during treatment planning based on a prescribed dose to a target region and imaging data of the target region (e.g., PET imaging data, an image of a target anchor, CT imaging data, etc.) and by iteratively solving for a fluence map that minimizes at least one cost function while still providing the prescribed dose to the target region. During a BgRT treatment session, the firing filters are applied (e.g., convolved) with the acquired imaging data to generate a corresponding radiation fluence. The radiotherapy system controller then segments (e.g., converts, translates) this radiation fluence into radiotherapy machine instructions (e.g., instructions for the MLC, linac, gantry, patient platform, etc.) so that the radiation fluence can be quickly delivered to the target region (e.g., in real-time, before the target region moves).

In one variation, BgRT uses imaging data obtained from positron emission tomography (PET) detectors to guide radiation. The PET image represents a PET signal from a tracer. For example, a PET image may be composed of a plurality of grey-scaled (or colored) pixels, with a shade (or color) of a pixel (herein, also referred to as the brightness or intensity of the pixel) corresponding to a concentration of a PET tracer in a given voxel of a tissue. For example, voxels that appear bright in a PET image, i.e., have a high intensity value, may indicate that the corresponding portion of tissue contains a high concentration of a PET tracer, while voxels that appear dimmer in a PET image, i.e., have a low intensity value, may indicate that the corresponding portion of tissue contains a low concentration of the PET tracer. In the examples described herein, the concentration of a PET tracer may also be referred to as a PET tracer activity concentration (AC), since the amount of positron emission activity in a tissue may correlate with the amount of PET tracer taken up by that tissue. The PET image may include a legend that maps colors (or gray-scale values) of the PET image to a number legend representing the concentration of the PET tracer. In various cases, a PET image may be obtained for different cross-sections of three-dimensional organs. Further, the PET image may be processed and depicted as three-dimensional surfaces and/or include contour lines representing constant concentrations of the PET tracer (e.g., iso-contours). PET images utilize tracer technology, and suitable PET tracers may include fluorodeoxyglucose (FDG). Examples of PET tracers, with their radioactive isotope in parentheses, may include acetate (C-11), choline (C-11), fluorodeoxyglucose (FDG) (F-18), sodium fluoride (F-18), fluoro-ethyl-spiperone (FESP) (F-18), methionine (C-11), prostate-specific membrane antigen (PSMA) (Ga-68), PSMA (Cu-64), DCFPyL (PyL) (F-18), DOTATOC, DOTANOC, DOTATATE (Ga-68), florbetaben (F-18), florbetapir (F-18), rubidium (Rb-82) chloride, ammonia (N-13), FDDNP (F-18), Oxygen-15 labeled water, FDOPA (F-18), fibroblast activation protein inhibitor (FAPI) (F-18), FAPI (Ga-68), FAPI (Cu-64), FAPI (Zr-89), fluoro-ethyl-tyrosine (FET) (F-18), anti-CD8 (Zr-89), fluoro-3′-deoxy-3′-L:-fluorothymidine (FLT) (F-18), PD-L1 (F-18), and flortanidazole (HX4) (F-18). Other tracers are known in the art as well. It should be appreciated that depending on a type of cancer, a particular tracer may be selected, and when one tracer is determined not to be suitable for BgRT procedure, another tracer may be used.

Disclosed herein are systems and methods for using a tracking filter, such as an adaptive correlation filter ACF, for BgRT. The ACF may be used for generating an estimate of the location of a patient tumor. The estimated location may be used for calculating a radiation fluence for delivery during a BgRT treatment session. A tracking filter (which may also be referred to ask a tracking template), such as an ACF, for a target region (e.g., a tumor) may be generated during a treatment planning session using one or more planning images, a target region contour defined on the planning images, and selected target anchor location. The planning images may comprise one or more of PET imaging data, CT imaging data, MR imaging data, X-ray imaging data, ultrasound imaging data, or combination thereof. During a treatment session, the tracking filter or template (e.g., ACF) may be applied to newly-acquired imaging data (e.g., PET imaging data, CT imaging data, MR imaging data) to generate an estimated location of the target anchor location. The estimated location of the target anchor location may be used to update the radiation fluence for delivery, which may help ensure that the prescribed radiation is delivered to the actual location of the target region. In some variations, an ACF may optionally be updated during the treatment session (e.g., at the start or multiple times throughout the treatment session), which may help further refine its estimation of the target anchor location (e.g., improve the accuracy and/or confidence of identifying the target anchor location).

### Systems

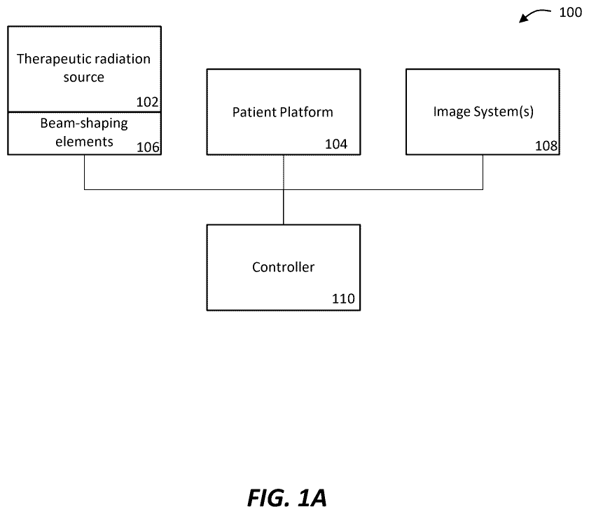

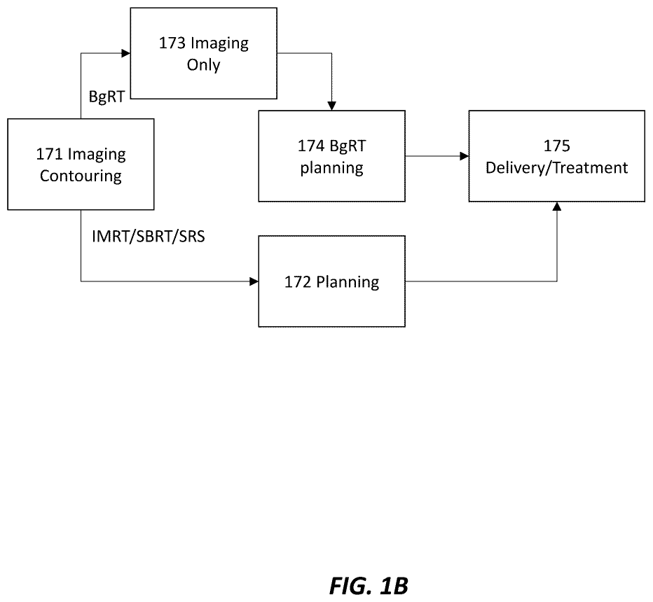

The methods described herein may be used with any type of image-guided radiotherapy, including but not limited to intensity-modulated radiotherapy (IMRT), stereotactic body radiotherapy (SBRT), stereotactic radiosurgery (SRS), and/or BgRT. Accordingly, the methods may be used with various radiotherapy systems, including, for example, a BgRT radiotherapy system (herein also referred to as a BgRT machine), which comprises PET detectors onboard with the therapeutic radiation source. FIG. 1A depicts a functional block diagram of a variation of a radiotherapy system that may be used with one or more of the methods described herein. Radiotherapy system 100 includes one or more therapeutic radiation sources 102 and a patient platform 104. The therapeutic radiation source may include an X-ray source, electron source, proton source, neutron source, carbon source or other particle or electromagnetic radiation source. For example, a therapeutic radiation source 102 may include a linear accelerator linac, Cobalt-60 source(s), and/or an X-ray machine. The therapeutic radiation source may be located on a gantry having a bore through which the patient platform is configured to move. The therapeutic radiation source may be movable about the patient platform so that radiation beams may be directed to a patient on the patient platform from multiple firing positions and/or firing angles. A firing position is the location of the therapeutic radiation source when it emits therapeutic radiation (e.g., a radiation beam or beamlet) to the patient area of the radiotherapy system. In the example of a radiotherapy system where the therapeutic radiation source moves around the patient platform in a single-plane (e.g., moving in a circular or arc trajectory within a X-Z plane along a Y-axis), the firing position may be referred to as a firing angle. In one variation, the system may continuously rotate from one firing angle to another or, alternatively, dwell at one or more specific firing angles for a period of time. In some variations, if the travel time from one firing position to the next is sufficiently short compared to the overall 360 degree rotation time, the dwell time at one firing position may include the travel time of the therapeutic radiation source to that firing position. In some variations, a radiotherapy system may include one or more beam-shaping elements and/or assemblies 106 that may be located in the beam path of the therapeutic radiation source. For example, a radiotherapy system may include a linac 102 and a beam-shaping assembly 106 disposed in a path of the radiation beam. The beam-shaping assembly may include one or more movable jaws and one or more collimators. At least one of the collimators may be a multi-leaf collimator (e.g., a binary multi-leaf collimator, a 2-D multi-leaf collimator, etc.). The linac and the beam-shaping assembly may be mounted on a gantry or movable support frame that includes a motion system configured to adjust the position of the linac to different firing positions about the patient platform and optionally, the beam-shaping assembly. In some variations, the linac and beam-shaping assembly may be mounted on a support structure comprising one or more robotic arms, C-arms, gimbals, and the like, with or without a bore. The patient platform 104 may also be movable. For example, the patient platform 104 may be configured to translate a patient linearly along a single axis of motion (e.g., along the IEC-Y axis), and/or may be configured to move the patient along multiple axes of motion (e.g., 2 or more degrees of freedom, 3 or more degrees of freedom, 4 or more degrees of freedom, 5 or more degrees of freedom, etc.). In some variations, a radiotherapy system may have a 5-DOF patient platform that is configured to move along the IEC-Y axis, the IEC-X axis, the IEC-Z axis, as well as pitch and yaw. Some systems may have a 6-DOF patient platform. The axis IEC-Y may be perpendicular to a bore of the gantry.

In the variation shown in FIG. 1A, radiotherapy system 100 also includes a controller 110 that is in communication with the therapeutic radiation source 102, beam-shaping elements or assemblies 106, patient platform 104, and one or more image systems 108 (e.g., one or more imaging systems).

Imaging systems 108 may include a PET imaging system which may be configured to obtain PET imaging data prior and/or during a BgRT therapy session. PET imaging data may also be acquired as part of a quality assurance session with a PET-avid phantom. In some variations, the PET imaging system includes a first array of PET detectors and a second array of PET detectors disposed across from the first array. The first and second arrays of PET detectors may be arranged as two arcs that are directly opposite to each other and may each have a 90° span around the patient treatment region. The PET detector arcs may not comprise an entire ring around the gantry; instead, they may be partial rings, where the therapeutic radiation source is located between the two partial rings or arcs. In some variations, the PET detectors may be time-of-flight PET detectors, which may help to identify the location of the positron annihilation event. Alternatively, or additionally, imaging systems 108 may include a CT imaging system such as a kV imaging system having a kV X-ray source and a kV detector. The kV detector may be located across the kV X-ray source. Optionally, the kV imaging system may include a dynamic multi-leaf collimator (MLC) disposed over the kV X-ray source. Additional details and examples of radiation therapy systems are described in U.S. application Ser. No. 15/814,222, filed Nov. 15, 2017, and PCT Appl. No. PCT/US2018/025252, filed Mar. 29, 2018, which are hereby incorporated by reference in their entireties. Alternatively, or additionally, imaging systems 108 may include a magnetic resonance imaging (MRI) system.

Controller 110 may include one or more processors and one or more machine-readable memories in communication with the one or more controller processors, which may be configured to execute or perform any of the methods described herein. The one or more machine-readable memories may store instructions to cause the processor to execute modules, processes and/or functions associated with the system, such as one or more treatment plans (e.g., BgRT treatment plans, SBRT/IMRT treatment plans, etc.), the calculation of radiation fluence maps based on treatment plan and/or clinical goals, segmentation of fluence maps into radiotherapy system instructions (e.g., instructions that may direct the operation of the gantry, therapeutic radiation source, beam-shaping assembly, patient platform, and/or any other components of a radiotherapy system), calculations for updating the location(s) of a target region (including, for example, the target anchor location), image and/or data processing associated with treatment planning and/or radiation delivery, updating adaptive correlation filters using newly-acquired imaging data, and/or generating estimates of the location of one or more target regions (including, for example, the target anchor location). In some variations, the memory may store treatment plan data (e.g., adaptive correlation filter(s), target anchor location(s), treatment plan firing filters, fluence map, planning images, treatment session PET prescan images and/or initial CT, MRI, and/or X-ray images), imaging data acquired by the imaging systems 108 before and during a treatment session, instructions for identifying the location of a target region and/or a target anchor using newly-acquired imaging data, and instructions for delivering the derived fluence map (e.g., instructions for operating the therapeutic radiation source, beam-shaping assembly and patient platform in concert). In some variations, one or more memories may also store PET metric values (e.g., metric values calculated using acquired data and/or threshold metric values), including, but not limited to one or more of contrast noise ratio (which may also be referred to as a normalized tumor signal), PET tracer activity concentration, and/or a radiation dose metric. These PET metric values may be used as part of a BgRT planning and treatment workflow to determine whether to proceed or to pause treatment. The controller of a radiotherapy system may be connected to other systems by wired or wireless communication channels. For example, the radiotherapy system controller may be in wired or wireless communication with a radiotherapy treatment planning system controller such that fluence maps, firing filters, initial and/or planning images (e.g., CT images, MRI images, PET images, 4-D CT images), patient data, simulated PET images, simulated line-of-responses (LORs) that correspond to a PET image, and other clinically-relevant information may be transferred from the radiotherapy treatment planning system to the radiotherapy system (herein, an LOR corresponds to a pair of co-linear, or nearly co-linear, 511 keV photons that travel in opposite directions from an annihilation event). The delivered radiation fluence, any dose calculations, and any clinically-relevant information and/or data acquired during the treatment session may be transferred from the radiotherapy system to the radiotherapy treatment planning system. This information may be used by the radiotherapy treatment planning system for adapting the treatment plan and/or adjusting delivery of radiation for a successive treatment session. In some variations, the radiotherapy treatment planning system may include a controller having one or more processors configured to perform the methods described herein, for example, methods for determining whether the BgRT metric values calculated from patient PET images meet threshold values that indicate BgRT may be appropriate. The radiotherapy treatment planning system may include one or more memories that store planning images (e.g., diagnostic PET images, simulated PET images, simulated LORs for a corresponding PET image, CT images, MR images), one or more contours for one or more target regions and/or organs-at-risk, anchor locations corresponding to the contours, adaptive correlation filters for each target region and/or organ-at-risk, target anchor locations, any of the BgRT metrics for any PET images, thresholds for the BgRT metrics, and the like. In some variations, the radiotherapy system and the radiotherapy treatment planning system may be integrated into a single system that performs both treatment planning and radiation delivery.

The patient platform 104 may be movable in a treatment region to discrete, pre-determined locations. These discrete, pre-determined locations may be referred to as “beam stations”. In one variation, different beam stations may vary only by their location along the IEC-Y axis (e.g., longitudinal axis); each beam station may be identified by its location along IEC-Y. Alternatively, or additionally, beam stations may vary by the platform pitch, yaw, and/or roll positions of the patient platform. For example, a radiotherapy treatment planning system may specify beam stations, where each beam station is about 2 mm (e.g., 2.1 mm) apart along IEC-Y from its adjacent beam stations. During a treatment session, the radiotherapy treatment system may move the patient platform to each of the beam stations and may stop the platform at a beam station while radiation is delivered to the patient. In some variations, after the platform has been stepped to each of the beam stations in a first direction (e.g., into the bore), the platform may be stepped to each of the beam stations in a second direction opposite the first direction (e.g., out of the bore, in reverse), where radiation is delivered to the patient while the platform is stopped at a beam station. Alternatively, or additionally, after the platform has been stepped to each of the beam stations in a first direction (e.g., into the bore) where radiation is delivered at each of the beam stations, the platform may be moved in reverse so that it returns to the first beam station. In some variations, no radiation may be delivered while the platform is moved back to the first beam station. The platform may then be stepped, for a second time, to each of the beam stations in the first direction for a second pass (referred to herein as a “shuttle pass”) of radiation delivery. Optionally, in some variations, radiation may be delivered while the platform is moved from the last beam station back to the first beam station. Some radiotherapy sessions may comprise multiple shuttle passes where the platform moves the patient through the treatment area (e.g., the region of the radiotherapy system where the platform is irradiated by the therapeutic radiation beam) multiple times. In some variations, the platform may be moved continuously while radiation is delivered to the patient and may not be stopped at beam stations during the delivery of therapeutic radiation. Additional descriptions of patient platforms that may be used with any of the radiotherapy systems and methods described herein are provided in U.S. Pat. No. 10,702,715, filed Nov. 15, 2017, which is hereby incorporated by reference in its entirety.

In some implementations, a distance between the beam stations may vary. For example, the beam stations may be more closely positioned to each other at a starting portion of a scanning section and at an ending portion of the scanning section. Alternatively, the beam stations may be more closely positioned to each other in a middle portion of the scanning section. In some variations, the distance between beam stations may be less than the width of a therapeutic radiation beam (or beamlet), such that the therapeutic radiation fields of adjacent beam stations may overlap with each other. Alternatively, or additionally, the distance between beam stations may be determined at least in part by the location of the target region on the patient platform, and/or the planned dose distribution. For example, platform positions that would place the patient within the treatment plane with a high dose gradient may have beam stations that are closer together, and while platform positions that would place the patient within the treatment plane with a low dose gradient (or no dose at all) may have beam stations that are further apart.

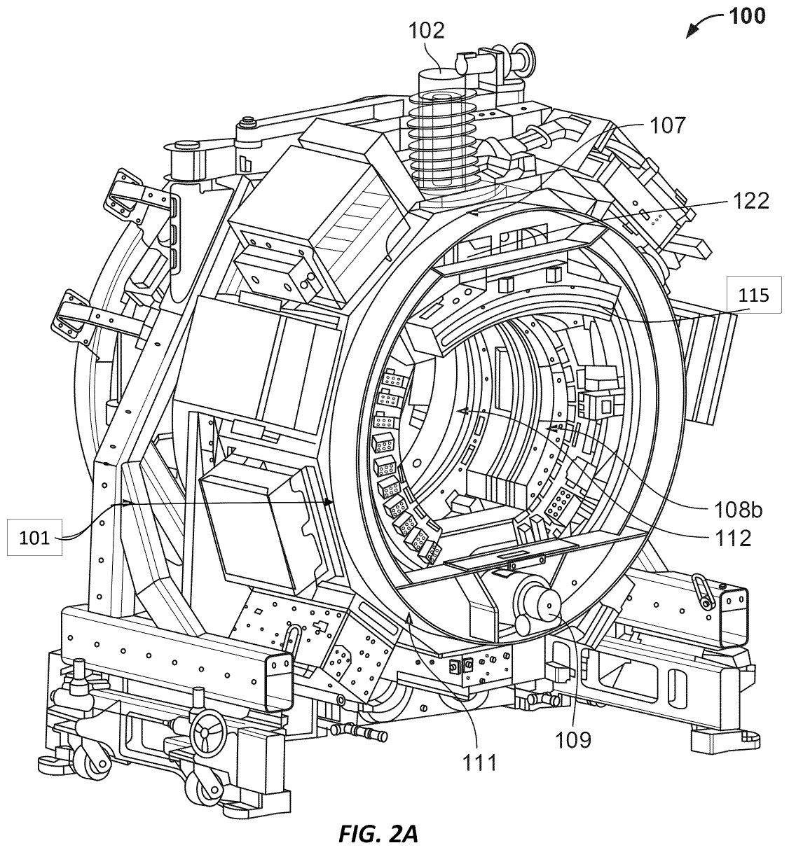

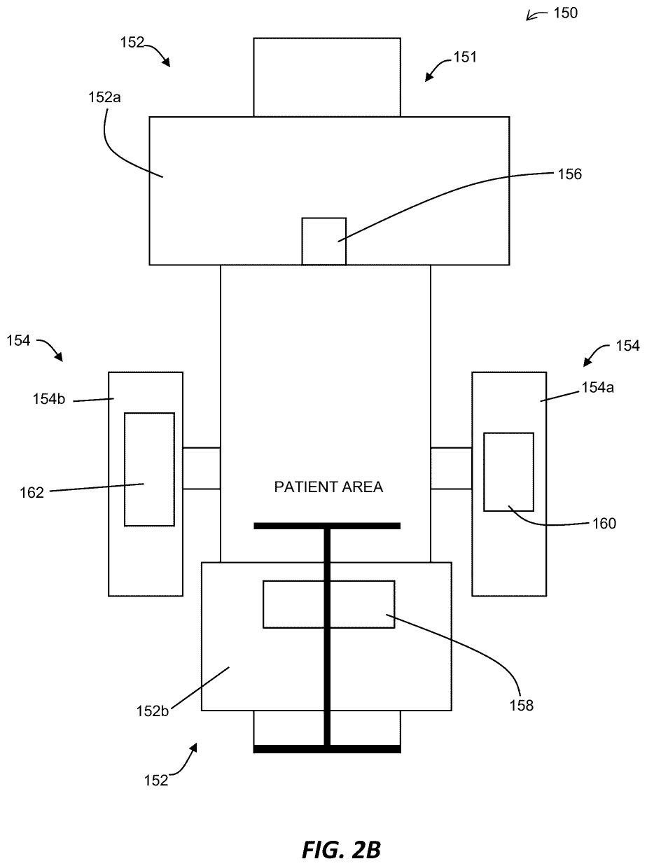

FIG. 2A depicts a perspective component view of the radiotherapy system 100. As shown there, the beam-shaping module may further include a primary collimator or jaw 107 disposed above the binary MLC 122. The radiotherapy system may also include an MV X-ray detector 103 located opposite the therapeutic radiation source 102. Optionally, the radiotherapy system 100 may further include a kV CT imaging system on a ring 111 that is attached to the rotatable gantry 101 such that rotating the gantry 101 also rotates the ring 111. The kV CT imaging system may include a kV X-ray source 109 and an X-ray detector 115 located across from the X-ray source 109. The therapeutic radiation source or linac 102 and the PET detectors 108a (not visible, but located directly across from 108b) and 108b may be mounted on the same cross-sectional plane of the gantry (i.e., PET detectors are co-planar with a treatment plane defined by the linac and the beam-shaping module), while the kV CT scanner and ring may be mounted on a different cross-sectional plane (i.e., not co-planar with the treatment plane). The radiotherapy system 100 of FIG. 2A may have a first imaging system that includes the kV CT imaging system and a second imaging system that includes the PET detectors. Optionally, a third imaging system may include the MV X-ray source (i.e., linac 102) and MV detector. The imaging data acquired by one or more of these imaging systems may include X-ray and/or PET imaging data, and the radiotherapy system controller may be configured to store the acquired imaging data and calculate a radiation delivery fluence using the imaging data, for example, in a BgRT session. Some variations may further include patient sensors, such as position sensors and the controller may be configured to receive location and/or motion data from the position sensor and incorporate this data with the imaging data to calculate a radiation delivery fluence. Additional descriptions of radiotherapy systems that may be used with any of the methods described herein are provided in U.S. Pat. No. 10,695,586, filed Nov. 15, 2017.

FIG. 2B depicts another variation of a radiotherapy system 150 that may be used to deliver radiation in accordance with any of the methods described herein. The radiotherapy system 150 may have the components of the radiotherapy system represented in the block diagram of FIG. 1. Radiotherapy system 150 may comprise a gantry or support structure 151 comprising a first pair of arms 152 rotatable about a patient area and a second pair of arms 154 rotatable about the patient area, an imaging system comprising a therapeutic radiation system comprising an MV radiation source 156 mounted on a first arm 152a of the first pair of arms 152 and an MV detector 158 mounted on a second arm 152b of the first pair of arms 152, and a kV radiation source (160 mounted on a first arm 154a of the second pair of arms 154 and a kV detector 162 mounted on a second arm 154b of the second pair of arms 154). The first and second arms of the first pair of arms 152 may be located opposite each other (e.g., on opposite sides of the patient area, across from each other, and/or about 180 degrees from each other), such that the MV radiation source 156 and the MV detector 158 are located opposite each other (e.g., the MV detector is located in the beam path of the MV radiation source). The first and second arms of the second pair of arms 154 may be located opposite each other (e.g., on opposite sides of the patient area, across from each other, and/or about 180 degrees from each other), such that the kV radiation source 160 and the kV detector 162 are located opposite each other (e.g., the kV detector is located in the beam path of the kV radiation source). The radiotherapy system controller may be configured to store acquired imaging data (from either or both the kV detector and the MV detector) and calculate a radiation delivery fluence. Optionally, one or more target region surrogate devices, such as a breathing sensor, may be included, and the controller may be configured to receive location and/or motion data from the target region surrogate to calculate a radiation delivery fluence.

FIG. 1B is a flowchart that depicts radiotherapy workflows for BgRT and SBRT/IMRT/SRS. Workflows for both BgRT and SBRT/IMRT may include imaging the patient and generating contours 171 for the target regions and organs-at-risk. An SBRT/IMRT workflow may include planning 172 and then radiation delivery 175. In contrast, a BgRT workflow may include an optional imaging-only session 173 where PET imaging data and CT imaging data are acquired on a BgRT radiotherapy system. The BgRT workflow may include incorporating this additional PET imaging data and/or CT imaging data into BgRT planning 174 and then proceeding with radiation delivery 175. Generating a BgRT plan may comprise one or more of generating firing filters for converting imaging data into radiation fluence, generating a radiation fluence map that is configured to deliver a prescribed dose, and/or generating an adaptive correlation filter. In some variations, a BgRT radiation delivery or treatment session may include a PET pre-scan, which may confirm that the PET tracer uptake by the patient on the day of treatment is satisfactory and/or safe for BgRT radiation delivery.

For both BgRT and IMRT/SBRT/SRS workflows, imaging and contouring 171 may include obtaining one or more diagnostic-quality (e.g., simulation) CT images, MR images, and/or PET images of a target region, and determining one or more contour lines of a planned target volume (PTV) as well as organs that may be radiation-sensitive (also referred to as organs-at-risk or OARs). For a BgRT workflow, contouring 171 may also comprise defining a biological guidance margin that accounts for positional uncertainties that may include one or more of tracking uncertainties and/or registration uncertainties between the simulation CT and the planning PET images. In some variations, a contour may be defined by expanding the PTV by the biological guidance margin. Alternatively, or additionally, contouring 171 for a BgRT workflow may comprise defining a biology-tracking zone (BTZ), which may be a region that encompasses the range of motion of the target region or PTV. For BgRT delivery, the contours of the BTZ may be the boundaries of a spatial filter used to select the PET imaging data that is to be used for guiding radiation delivery. Contours may be defined to include PET-avid regions, such as PET-avid tumors and PET-avid OARs. OAR contours may be used during treatment planning as a dose constraint (i.e., ensuring that no more than a threshold amount of radiation may be delivered to the OAR) and/or during a radiation delivery session to support tracking the location of the OAR and avoid irradiating it. For example, during a radiation delivery session, if the location of the OAR is within a predefined distance to the target, radiation may not be delivered to the target because it may also irradiate the OAR.

For an IMRT/SBRT/SRS workflow, treatment planning 172 may comprise generating a planned fluence map that is configured to deliver the dose prescribed by the clinician. Optionally, IMRT/SBRT/SRS treatment planning may include segmenting the planned fluence map into radiotherapy system machine instructions so that the prescribed radiation dose is delivered as long as the patient is properly localized and the radiotherapy system successfully executes the machine instructions during the treatment session 175. Alternatively, or additionally, instead of segmenting the planned fluence map into radiotherapy machine instructions during treatment planning, an IMRT/SBRT/SRS workflow may include segmenting the planned fluence map during the treatment session 175 (i.e., real-time segmentation).

In contrast to the IMRT/SBRT/SRS workflow, a BgRT workflow may include an additional imaging-only session 173 where additional PET and CT imaging data is acquired on the BgRT radiotherapy system. The imaging only step 173 may be similar to a process of obtaining PET and CT images during a BgRT treatment session. In some variations, the imaging-only session 173 may have a similar time duration as a BgRT treatment session.

In some variations, BgRT planning 174 may comprise defining the desired dose distribution, and subsequently, calculating a mapping function that relates the planning PET image to the desired dose distribution. This mapping function may define a set of firing filters that, when applied to PET imaging data, converts the imaging data into a corresponding radiation fluence for delivery. In some variations, BgRT treatment planning may comprise generating a planned fluence map and firing filter(s) based on CT imaging data, PET imaging data, and/or an image of a target anchor (as described further below). The planned fluence map and the firing filters generated during BgRT planning 174 may be transferred to the radiotherapy system for BgRT delivery 175. In contrast to IMRT/SBRT/SRS treatment planning, the output of a BgRT treatment plan does not include radiotherapy machine instructions for executing during a treatment session, but instead outputs firing filters which are applied to PET imaging data acquired during the BgRT treatment session to calculate a radiation fluence for delivery. The calculated radiation fluence is segmented into machine instructions in real-time during the treatment session.

The methods described herein comprise applying a tracking filter, such as an adaptive correlation filter (ACF), to imaging data acquired during a radiotherapy session and using the filtered image to guide the delivery of radiation. Any of these methods may be used with IMRT/SBRT/SRS and/or BgRT, which is a type of radiotherapy that converts biologically-related imaging data acquired on the day of treatment into radiation fluences for delivery. Examples of biologically-related imaging data may include, but is not limited to, metabolic data, cellular receptor data, intracellular pressure, cellular density, cellular diffusion properties, and the like. The acquisition time for the imaging data may be relatively short (e.g., about 60 seconds or less, about 10 seconds or less, about 5 seconds or less, about 3 seconds or less, about 1 second or less, about 0.5 second or less, etc.) so that the BgRT radiotherapy system can quickly respond with radiation beamlets before the target region moves substantially. However, because the radiation fluence is calculated from limited-time sampled imaging data, there may be unwanted imaging artifacts and/or noise that convert into radiation fluence with corresponding artifacts and/or noise. This may negatively impact the conformality of the radiation fluence and may result in over-irradiating surrounding healthy tissue and/or under-irradiating the target region. One approach that has been used to reduce imaging artifacts and/or noise comprises using a difference of Gaussians (DoG) method to filter to the imaging data. The filtered imaging data is then used to generate the radiation fluences for delivery. However, there are some artifacts and/or noise that are not adequately suppressed or eliminated using the DOG method, and may result in generating undeliverable (e.g., negative) fluence values.

The methods described herein reduce the effects of imaging noise and artifacts by applying a tracking filter, such as an adaptive correlation filter (ACF), to imaging data. In some variations, applying the tracking filter to imaging data may comprise convolving the tracking filter with imaging data. The ACF may be an imaging template of a target region that may be applied to imaging data to identify the target region within that image. Identifying the target region using an ACF may include determining the location of the target region within an image that has been filtered by the ACF. For example, in some variations, using an ACF to process imaging data may generate an estimated location of a target region anchor. In other words, filtering or processing the imaging data using an ACF may generate a filtered image that has a high-intensity region at the location of the target anchor location. Conceptually, applying an ACF template to imaging data may be similar to pattern matching; the ACF template may be shifted across the imaging data to find the region of the image that correlates most strongly with the “template” pattern (e.g., highly correlated with an image of the tumor). Once the region of the image that correlates strongly with the planning image of the tumor, that region is “highlighted” while regions with lower correlation are suppressed or ignored. In other words, the noise and/or artifacts in the original imaging data may be eliminated or suppressed in the filtered image of the target anchor location. The anchor of a target region is a location that is defined relative to a contour of the target region during treatment planning. During treatment planning, firing filters may be generated based on CT imaging data, PET imaging data, and/or one or more images of the target region anchor (e.g., a Gaussian or delta function centered over the defined location of the target region anchor). During radiation delivery, the firing filters may be applied to the estimated location of the target anchor to generate a radiation fluence for delivery. The estimated location of the target anchor may be determined from imaging data that has been filtered (and/or cross-correlated) with the ACF, and an image of the target anchor location may comprise a Gaussian or delta function centered over the estimated location of the target anchor. Because the firing filters are applied to an image of the target anchor location, and not to the imaging data directly, the resultant radiation fluence may be less susceptible to the effects of imaging noise and artifacts. In some variations, the image of the target anchor location may be a de-noised version of the filtered image, where the region of the image that corresponds with the location of the anchor is sharpened or intensified, and the image noise is suppressed. The image of the target anchor location may be a combination of empirically acquired imaging data and simulated imaging data, for example, a Gaussian function (simulated data) centered over the location of the anchor and suppressed imaging noise from the acquired imaging data. Alternatively, or additionally, the planned radiation fluence may be shifted according to the estimated location of the target region anchor. Moreover, in some variations, the ACF may be trained with additional imaging data (i.e., further refining the imaging template) so that it provides a better estimate of the target anchor location.

In one variation of a method for generating an estimate of the target anchor location during a treatment session, the ACF calculated during treatment planning may be updated using additional imaging data before the treatment session and optionally, using imaging data acquired at the beginning of the session. For example, after the ACF is generated during treatment planning, it may be further adapted or updated (i.e., trained) using imaging data that has both the target contour and the target anchor location. Examples of such imaging data may include synthetic imaging data that may simulate variations in the imaging data on the day of treatment, such as different noise levels, location shifts in the target region, geometric changes in the target region, and/or fluctuations in the imaging signal intensity (e.g., brightness) from the target region. For PET imaging data, there may be variations in PET tracer uptake by the target region, and these variations in the tracer uptake characteristics may be simulated and included in the synthetic imaging data.

Some methods may comprise acquiring additional imaging data on the day of treatment, for example, localization CT images and/or PET images, and/or any prescan images that are acquired before radiation is applied to the patient. The target contour and target anchor location may be identified on these additional images and then the images may be used to further update or adapt the ACF before radiation delivery. This may update or adapt the ACF to incorporate the characteristics of the imaging data and/or condition of the patient on the day of treatment, which may in turn facilitate an improved estimation of the target anchor location. Alternatively, or additionally, the ACF may be further updated or adapted (e.g., trained) at one or more time points during a treatment session as new imaging data is acquired. In some variations, the tracking filter or template may be defined during treatment planning and/or at the beginning of a treatment session, and not updated or trained on additional imaging data.

While the methods are depicted and described BgRT workflows in some examples, it should be understood that these methods may also be used in an IMRT/SBRT/SRS workflow (which may also be referred to as an image-guided radiotherapy workflow) as depicted and described in other examples. In the context of treating multiple target regions in one treatment session, some variations may comprise treating some target regions using BgRT while treating other target regions using IMRT/SBRT/SRS, and the methods described herein may be combined in order to do so, as may be desirable by the clinician.

The examples provided herein use an adaptive correlation filter as a tracking filter to determine an estimated location of a target region and/or target anchor, however, it should be understood that in some variations, any tracking filter or template may be used to determine an estimated location of the target region.

The anchor of a target region (which may also be referred to as a target anchor or a target region anchor or anchor point) is a location that is associated with a target region contour defined during treatment planning. A target region is a volume of a patient that is to receive a prescribed dose of radiation. The volume of a target region is defined by a 3-D contour, and 2-D sections or slices will have a corresponding 2-D contour. The target region contour and its corresponding target anchor may be defined using one or more planning images (e.g., CT images, PET images, MR images, X-ray images, ultrasound images, etc.). In some variations, the target region contour may be the boundary of a PET “cloud” (or PET-avid region) of a PET image that represents the elevated PET tracer uptake by a tumor. In some variations, it may be desirable to track the location and/or motion of PET-avid OARs, in which case, an OAR region contour may be defined as the boundary of the PET cloud corresponding to the elevated PET tracer uptake by an OAR. Conceptually, the target anchor location is a “landmark” for a target region that may be used during a treatment session as a reference point (i.e., “anchor”) for radiation fluence delivery. In some variations, the planned radiation fluence is generated with the target anchor location as its reference point so that during the treatment session, once the location of the anchor point is identified, the planned radiation fluence may be delivered to the location of the target region that may be determined from the anchor point. In some variations, the location of the anchor point may be represented or depicted in an image comprising one or more pixels or voxels. The location of the target anchor may be represented in an image as one or more pixels or voxels of an anchor function. For example, an anchor function may be defined based on a location of the anchor point (e.g., a delta function or a Gaussian function centered on the anchor point location, a plurality of delta functions or a plurality of Gaussian functions located around in proximity to the anchor point location), and an image of the anchor function may be determined numerically as an image representation of a numerical delta function or a Gaussian function (or a plurality of numerical delta functions or plurality of Gaussian functions). The target anchor location may be represented as a single pixel or voxel in an image (e.g., a delta function of a single pixel or voxel that is at the target anchor location) and/or multiple pixels or voxels in an image that have a centroid at the target anchor location (e.g., a Gaussian function comprising a cluster of multiple pixels or voxels centered at the target anchor location). In some variations, the image of the target anchor location may be a de-noised version of the imaging data that has been filtered by an ACF, where the region of the image that corresponds with the location of the anchor is sharpened or intensified, and the image noise is suppressed. The image of the target anchor location may be a combination of empirically acquired imaging data and simulated imaging data, for example, a Gaussian function (simulated data) centered over the location of the anchor and suppressed imaging noise from the acquired imaging data. Alternatively, in some variations, the image of the target anchor location may be the filtered and/or cross-correlation image.

It should be understood that similar to target regions, OARs can have a defined contour and corresponding reference point or anchor. In contrast to the target anchor which helps a radiotherapy system determine where to deliver high doses of radiation, an OAR anchor may be used to determine patient regions where irradiation is to be avoided or reduced. The OAR contour and relative location of an OAR anchor may be determined and used to generate an ACF during treatment planning. The ACF may then be applied to imaging data acquired during a treatment session to identify the location of the OAR anchor and generate an estimate of the OAR contour location. If the OAR contour and/or OAR anchor location is within a predetermined distance to a target region, or is in a position where it may receive more radiation than intended, a notification may be generated, and radiation delivery may be paused. In BgRT, some OARs may be PET-avid (i.e., accumulate elevated levels of PET tracer as compared to background) and the ACF may be used to determine its location based on acquired PET imaging data.

During treatment planning, a clinician may define the target region using one or more planning images by defining the contours or boundaries around the target region. In addition to defining the contour of the target region, the clinician may also select the location of the target anchor. The relative position of the target anchor to the target region contour may be determined during treatment planning and transferred to the radiotherapy system controller along with the other treatment plan data. As described above, the target region contour may be the boundary of a PET-avid target region. In some variations, the target region contour may be a gross tumor volume (GTV), a clinical target volume (CTV), an internal target volume (ITV), a planning target volume (PTV), a biology guidance margin (BgM), and/or a biology-tracking zone (BTZ). These contours may or may not approximate the boundary of a PET-avid target region. In some variations, these methods may be used to contour OAR, which represents a patient region where irradiation should be limited or avoided. For example, in one of several ways to avoid irradiating the OAR, the fluence can track relative anchor point defined in a PET-avid OAR, but still cover the clinical target volume (CTV) with sufficient dose. Some methods may comprise storing the location of the OAR contour and/or OAR anchor and if one or both are located at an acceptable margin away from the target contour and/or target anchor, radiation delivery to the target region may continue. The anchor point may be selected to be a location within the target region contour, on the boundary of the target region contour, or in some variations, outside the target region contour. In some variations, the target anchor location identifies a substantially central point of the PTV (e.g., may be a centroid). For example, the location of the anchor point may be at a centroid location of the target region. Alternatively, the anchor location may be on a selected anatomical structure that may be coupled to the target region, such as an anatomical structure whose position is associated with the position of the target region. For example, for tumor that is associated with (e.g., coupled to) a bone, the anchor location may be selected to be on a point of that bone, which may or may not be within the boundaries of the tumor. In general, the anchor location may be selected to be the location of any distinct feature associated with the PTV contour (e.g., a sharp corner, a bump, a distinct shape, and the like) that may be readily identifiable or distinguished from other features in an image of the target region.

The methods described herein use an adaptive correlation filter (ACF) to facilitate determining the location of the target anchor during a treatment session, using the imaging data (e.g., CT imaging data, PET imaging data, MR imaging data, etc.) acquired during the treatment session. An ACF is a filter that highlights the portion of an image that corresponds to a desired region of interest. In other words, the ACF may be a “template” that is used to identify regions within an image that match (within an acceptable tolerance or variance) the template. In some variations, the ACF is a filter that identifies the portion of an image that corresponds to the location of the target anchor. The ACF may be defined during treatment planning based on the target region contour and the target anchor location defined on planning images, and it may optionally be updated or adapted using additional imaging data. The additional updates or adaptations may help the ACF generate a correlation image (i.e., an image that represents the correlation of newly-acquired imaging data with the images and/or imaging data used to generate the ACF) that facilitates a more accurate estimate of the target anchor location on the day of treatment. In some variations, applying the ACF to an image and/or imaging data may comprise cross-correlating the imaging data with the ACF to generate a correlation image (which may be referred to herein as a filtered image). For example, a digital representation of a PET image or CT image may be a two-dimensional matrix f, in which matrix elements represent greyscale (or color) values corresponding to the PET image or the CT image. Similarly, the ACF may be represented as a two-dimensional matrix h. To generate the filtered image, the ACF h is applied to the image f. In one implementation, applying ACF h to image f includes cross-correlating h with image f. The cross-correlation of h and f may be represented as c=fêh (here, the operator ê denotes the cross-correlation operation). The cross-correlation c may also be determined by taking a Fourier transform of image f as F=FT(f), and the complex conjugate of the Fourier transform of matrix h as H*=[FT(h)]*, resulting in two-dimensional matrices Fᵢⱼ and Hᵢⱼ*. The Fourier transform of a cross-correlation, C, then is calculated by multiplying pointwise the matrices F and H* as Cᵢⱼ=F⊙H*=FᵢⱼHᵢⱼ*, and the cross-correlation c is calculated as an inverse Fourier transform c=FT⁻¹(C). Here, symbol @ is used to explicitly denote element-wise multiplication.

In general, for a given image (e.g., a PET image) a suitable filter h for determining a target anchor position for the tumor is configured to generate a suitable Gaussian function g centered at the target anchor position when f is cross-correlated with h, i.e., c=fêh=g. Thus, for a particular image f the filtering function h may be determined for a corresponding selected Gaussian function g at the target anchor location. Thus, determining the ACF h reduces in finding h that satisfies fêh=g, with f and g being known quantities. Finding h may be facilitated using Fourier transforms. For instance, for

$$G = F T ( g ) , {H}_{i j}^{*} = {G}_{i j} / {F}_{i j} , with h = F {T}^{- 1} ( {H}^{*} ) ,$$

### and F=FT (f).

While the examples and methods described herein are in the context of BgRT involving PET imaging data, it should be understood that the same methods may be used for BgRT involving imaging data of other modalities, including, but not limited to CT imaging data, MR imaging data, optical imaging data, and/or ultrasound imaging data. Moreover, although the examples provided herein are described in the context of a radiotherapy treatment session, it should be understood that the systems and methods may be used during a non-treatment session, i.e., in the absence of a patient. For example, any of the systems and methods described herein may be used in a quality assurance (QA) and/or calibration session with a phantom in the treatment area instead of a patient. In some variations, the phantom may comprise one or more target regions that have positron emission activity that may mimic the uptake of PET tracer in a patient. Alternatively, or additionally, a radiation measurement device may be positioned in the treatment area. A treatment plan may be delivered and the phantom and/or radiation measurement device may be used to evaluate whether the radiotherapy system functioned properly to deliver the treatment plan. In the case of BgRT, the treatment plan may include firing filters and an ACF for each target region, and the methods described herein may be used to generate a radiation fluence for delivery during a QA and/or calibration session.

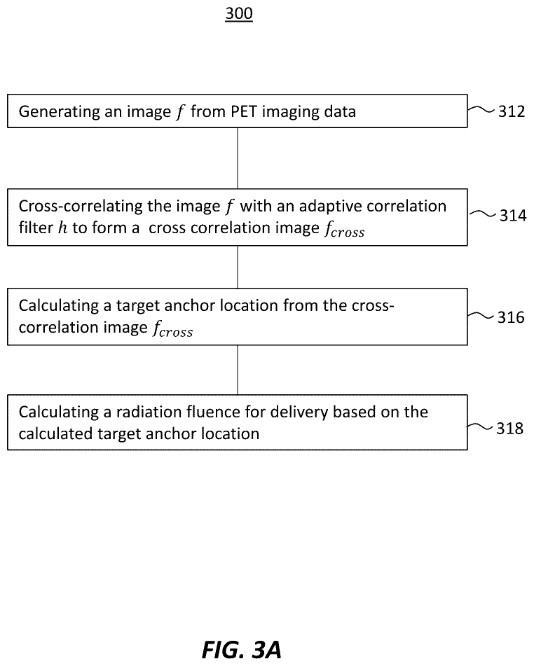

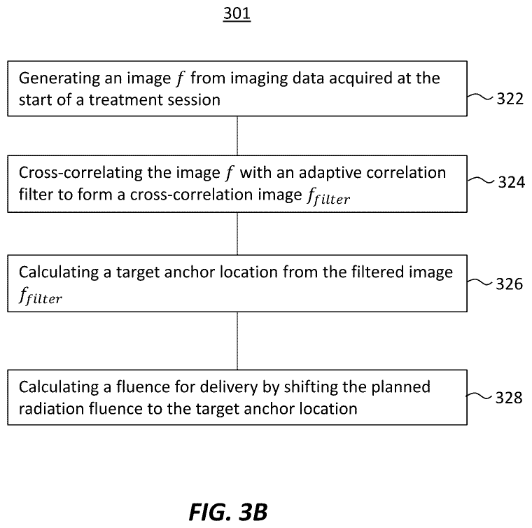

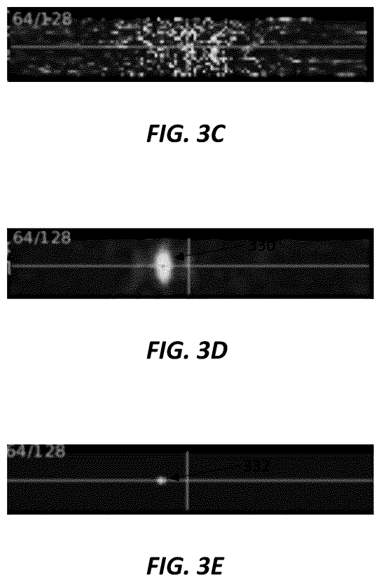

FIG. 3A depicts a flowchart representation of one variation of a method for calculating a target anchor location and radiation fluence for delivery using an adaptive correlation filter during a BgRT treatment session. The method 300 includes generating 312 an image f from acquired PET imaging data, cross-correlating 314 the image f with an adaptive correlation filter to form a cross-correlation image, calculating 316 a target anchor location from the cross-correlation image, and calculating 318 a radiation fluence for delivery based on the calculated target anchor location. The PET imaging data may be acquired at a particular beam station or control point, e.g., patient platform position. The image f may be a two-dimensional matrix as described above. Optionally, method 300 may comprise removing noise from the image f, for example, using a generative adversarial network (GAN) and/or one or more filters such as low-pass filters. In some variations, cross-correlating 314 the image f may comprise cross-correlating the image matrix f with the ACF h. The cross-correlation image may also be referred to as a filtered image. In one variation, the target anchor location may correspond to indices i, j of f_{filter}(i, j) (herein, such indices are also referred to as coordinates) at which the filtered image has a maximum value (e.g., {i, j}→max (f_{filter}(i, j)). Calculating or determining 316 the target anchor location from the cross-correlation image may comprise by calculating a centroid of the filtered (i.e., cross-correlation) image and/or a centroid of a region of pixels or voxels that have an intensity value above a specified threshold. Alternatively, or additionally, calculating the target anchor location may comprise averaging the location of pixels that have an intensity value above a specified threshold. For example, calculating 316 the target anchor location may include identifying the high-intensity pixel(s) or voxel(s) in the cross-correlation image f_{filter}. The location of the target anchor may be the location of the highest-intensity pixel or voxel, and/or may be the location of the centroid of a cluster of high-intensity pixels or voxels. Method 300 may optionally comprise generating an image of the target anchor location, and using the image of the target anchor location to calculate 318 the radiation fluence for delivery. An image of a target anchor location may be a Gaussian function g (or a function similar to a Gaussian function, or a delta function) which may have a maximum value at (or in close proximity of) the target anchor location. In some variations, the image of a target anchor location may be an image of a delta function where the peak or maximum value is centered on the target anchor location determined from the cross-correlation image. Alternatively, or additionally, the image of a target anchor location may be an image having a plurality of delta functions where the peaks are in proximity to the target anchor location. The characteristics of the Gaussian function and/or delta function used to generate the image of the target anchor location may be the same as the Gaussian function and/or delta function used during treatment planning. For example, if treatment planning was based on an image of the target anchor that comprises a Gaussian function of a particular RMS width (or spread) and peak located over the location of the target anchor, generating an image of the target anchor location during radiation delivery may use the same Gaussian function with the same width and peak. In some variations, the image of the target anchor location may be a de-noised version of the filtered image, where the region of the image that corresponds with the location of the anchor is sharpened or intensified, and the image noise is suppressed. The image of the target anchor location may be a combination of empirically acquired imaging data and simulated imaging data, for example, a Gaussian function (simulated data) centered over the location of the anchor and suppressed imaging noise from the acquired imaging data.

For example, one variation of the method 300 may comprise acquiring imaging data (e.g. PET imaging data), generating a filtered image by applying (or cross-correlating) an ACF with the acquired imaging data, calculating the target anchor location by identifying the location(s) of the high-intensity pixels or voxels in the filtered image, generating an image of the target anchor location by using a Gaussian or delta function centered over the calculated target anchor location, and calculating the radiation fluence for delivery using the image of the target anchor location (e.g., by convolving the image of the target anchor location with firing filters, shifting the planned radiation fluence to the target anchor location). Optionally, before the applying the ACF to the acquired imaging data, the noise in the imaging data may be removed or reduced using, for example, a denoising GAN model (e.g., a GAN that has been trained on multiple prior images of the patient and target region(s)) and/or one or more noise-reduction filters.

In one variation, calculating 318 the radiation fluence for delivery may include applying a firing filter to the target anchor location and/or the image of the target anchor location. As described previously, a firing filter may be a mapping function that converts imaging data (e.g., an image of the target anchor location) into a corresponding radiation fluence. The firing filter may be calculated based on previously-acquired imaging data of the target region (e.g., CT imaging data, PET imaging data, and/or an image of the target anchor) during treatment planning, as described above. In some variations, applying the firing filter to the target anchor location comprises convolving an image of the target anchor location (e.g., a Gaussian function and/or a delta function centered over the location of the target anchor) with the firing filters. Alternatively, or additionally, the firing filter may be applied by convolving the filtered image fritter with the firing filter. An example description of calculating firing filters during treatment planning and applying the calculated firing filters to a suitable image (e.g., the filtered image f_{filter} or a PET image of the target region or an image of the target anchor location) is provided in U.S. Pat. No. 10,688,320 filed May 30, 2018, and International Patent Application No. PCT/US2021/044405, filed Aug. 3, 2021, which are hereby incorporated by reference in their entirety. In some variations, calculating the radiation fluence includes shifting a planned radiation fluence according to the target anchor location. For example, the planned radiation fluence may be defined relative to the target anchor location based on planning images during treatment planning. Prior to radiation delivery, the location of the target region and the target anchor location may have changed. The updated location of the target anchor may be calculated by applying the ACF to the imaging data, as described herein. Then, for the radiation delivery, the planned radiation fluence may be shifted to the calculated target anchor location. That is, the changes and shifts of the target anchor location from the planning phase to the treatment session may be applied to the planned radiation fluence so that the radiation is delivered to the updated location of the target. For example, if the target anchor location shifted by 1 cm, the radiation fluence for delivery may be calculated by shifting the planned radiation fluence by 1 cm. In some cases, the planned radiation fluence comprises a segmented multi-leaf collimator (MLC) leaf pattern and calculating the delivery radiation fluence comprises shifting the segmented MLC leaf pattern according to the calculated target anchor position. Alternatively, or additionally, calculating the radiation fluence for delivery may comprise using a lookup table that maps anchor target locations to corresponding MLC leaf patterns, and updating the MLC leaf pattern based on the lookup table data. In some variations, the methods described herein may be used to track changes in the target anchor location before and/or during a treatment session, and then apply those changes (e.g., positional shifts) to the radiation fluence for delivery and/or machine instructions. Since the relative location between the target anchor and the target region contour may be defined during treatment planning, changes in the target anchor location may be used to estimate changes in the position of the target region. Adjusting the radiation fluence for delivery according to changes in the target anchor location may facilitate the delivery of the prescribed dose of radiation to the actual location of target region.

Optionally, the method 300 further includes delivering the calculated radiation fluence to the target region with a therapeutic radiation source.

During a BgRT treatment session, PET imaging data may be continuously acquired, even as radiation is delivered to the target region. In some variations, method 300 may be repeated for multiple times during the treatment session, to multiple collections or boluses of PET imaging data. In some variations, the target anchor location may be repeated calculated, i.e., updated, based on these collections of PET imaging data over the course of the BgRT treatment session. This may help to ensure that the delivered radiation tracks the actual location of the target region.

FIG. 3B depicts a flowchart representation of one variation of a method for calculating a target anchor location and radiation fluence for delivery using an adaptive correlation filter during an IMRT or SBRT treatment session. In some variations of IMRT or SBRT treatment sessions, no PET imaging data is acquired and/or no biologically-related imaging data is acquired. Method 301 may include generating 322 an image f from imaging data (e.g., CT images) acquired during the SBRT/IMRT procedure, cross-correlating 324 the image f with an adaptive correlation filter to form a cross correlation image, calculating 326 a target anchor location from the cross-correlation image, and calculating 328 a radiation fluence for delivery based on the calculated target anchor location. Optionally, method 301 may comprise removing noise from the image f, for example, using a generative adversarial network (GAN) and/or one or more filters such as low-pass filters. The imaging data may be acquired at the start of the treatment session and may comprise, for example, CT imaging data acquired to localize the patient on the patient platform (i.e., register the location of the patient relative to the coordinate system of the radiotherapy system). The image f is a two-dimensional matrix as described above. In some variations, cross-correlating 324 the image f may comprise cross-correlating the image f with the ACF h. As described above, the cross-correlation image may also be referred to as a filtered image (and an image filtered by an adaptive correlation filter may be referred to as a cross-correlation image). i, j of f_{filter}(i, j) (herein, such indices are also referred to as coordinates) at which the filtered image has a maximum value (e.g., {i, j}=→max (f_{filter}(i, j)). Alternatively, any other suitable approaches may be used for determining the target anchor location based on the filtered image f_{filter}. For example, the target anchor location may be a centroid of the filtered image and/or a region of image pixels that have an intensity value above a specified threshold. In some variations, an image of the target anchor location may be generated using the cross-correlation image f_{filter}.

The method 301 further includes calculating 328 a radiation fluence to be delivered to the target region based on the calculated target anchor location. In one implementation, the radiation fluence may be calculated by applying a firing filter to an image of the target anchor location, as described above. Alternatively, or additionally, the radiation fluence may be calculated by shifting the planned radiation fluence to the target anchor location.

Optionally, the method 301 further includes delivering the calculated radiation fluence to the target region with a therapeutic radiation source.

During an IMRT or SBRT or BgRT treatment session, imaging data may be acquired not just at the beginning of the treatment session, but optionally at one or more time points after radiation delivery has begun. In some variations, method 301 may be repeated multiple times during the treatment session, to multiple collections or boluses of imaging data. This may help to ensure that the target anchor location is routinely updated throughout the treatment session so that the delivered radiation tracks the actual location of the target region. For example, methods 300 or 301 may be repeated after each patient platform shuttle pass, where imaging data acquired during the shuttle pass and/or at the end of the shuttle pass is used to update the location of the target anchor. The updated location of the target anchor may then be used to calculate the radiation fluence for delivery in the next shuttle pass.

FIGS. 3C-3D depict simulation results of applying the method 300 to PET imaging data of a target region. FIG. 3C depicts PET imaging data that was acquired over a short time frame (e.g., less than 1 second) and contains imaging artifacts and elevated levels of noise. Although PET imaging data is used in this simulation, it should be understood that the same simulation may use imaging data from other imaging modalities, such as imaging data from X-ray imaging, MR imaging, CT imaging, ultrasound imaging, etc., in accordance with method 301. An adaptive correlation filter (ACF) was previously-defined based on higher-quality (e.g., lower-noise, stronger signal) PET imaging data of the target region. For example, the ACF may have been generated based on a lower-noise PET image of the target region (e.g., high signal-to-noise ratio, fewer imaging artifacts, such as a diagnostic-quality PET image). The ACF was applied to the imaging data depicted in FIG. 3C to attain the filtered image (i.e., cross-correlation image), which is depicted in FIG. 3D. The target anchor location was determined from the filtered image of FIG. 3D by calculating the centroid of the region 330 of the image having high-intensity pixels. FIG. 3E depicts an image of the target anchor location which contains a Gaussian function (e.g., a Gaussian with a narrow spread or RMS width, or in some variations, a delta function) that is centered over the location 332 of the target anchor. A function or distribution centered over the target anchor location may also be referred to as an anchor function. The image of the anchor location depicted in FIG. 3E may then be used to calculate the radiation fluence for delivery, for example, by convolving the image of the anchor location with a firing filter and/or by shifting the planned radiation fluence to the anchor location (i.e., from its original location during treatment planning). As demonstrated in this simulation, the noise and/or artifacts in the original imaging data depicted in FIG. 3C (e.g., scatter, random photon events, glare, etc.) have been reduced or removed using the ACF and then further “compressed” into a narrow Gaussian or delta function at the location of the target anchor location. Using a sharp, relatively noise-free or low-noise imaging signal to calculate radiation fluence for delivery may reduce or eliminate dose artifacts that originate from a noisy image, and may help facilitate the delivery of a radiation dose that better conforms with the prescribed dose distribution.

The examples provided herein use an adaptive correlation filter as a tracking filter to determine an estimated location of a target region, however, it should be understood that in some variations, any tracking filter or template or correlation filter may be used to determine an estimated location of the target region. For example, the tracking filter or template or correlation filter may be defined during treatment planning and/or at the beginning of a treatment session, and not updated or trained on additional imaging data. The tracking filter or correlation filter, without any further training or updates, may be applied to imaging data acquired during a radiation delivery session.

An adaptive correlation filter may be generated using an initial set of imaging data and then further updated (i.e., adapted, trained) based on additional imaging data. For radiotherapy methods, the ACF may be generated based on target region contours that are defined using planning images (e.g., CT, MR, and/or PET images) and a target anchor for each target region, both defined at least in part by a clinician (with or without the aid of auto-contouring tools). For example, the target region contour and target anchor (including its location) may be defined on planning CT images. The relative position between the target anchor and the target region contour may be fixed during treatment planning. The treatment planning system may then calculate a planned fluence that is associated with the target region contour and is referenced to the target anchor. That is, the target anchor functions as a reference point or landmark, and the planned radiation fluence may be associated or “locked onto” the target anchor. During treatment delivery, the location of the target anchor may be determined using any of the methods described herein, and the planned radiation fluence is then adjusted to the target anchor location.

In the case of treatment planning for BgRT, the firing filters may be calculated based on an image of the anchor location (or an image of an anchor function derived from the anchor location), instead of PET images. That is, instead of generating firing filters by optimizing for a prescribed radiation dose as associated with PET imaging data, firing filters are generated by optimizing for a prescribed radiation dose associated with an image of the target anchor location (where optimization includes the process of minimizing at least one cost function or constraint on dose to attain the prescribed radiation dose). In some variations, an ACF may be generated based on a target region contour and anchor defined using planning CT images and planning PET images (or any biologically-related imaging data) aligned to the planning CT images. Planning PET images may be aligned to the planning CT images by, for example, aligning the high-intensity regions of the PET image (or any region of the PET image that is likely to be the target region) with the target anchor location on the planning CT images.

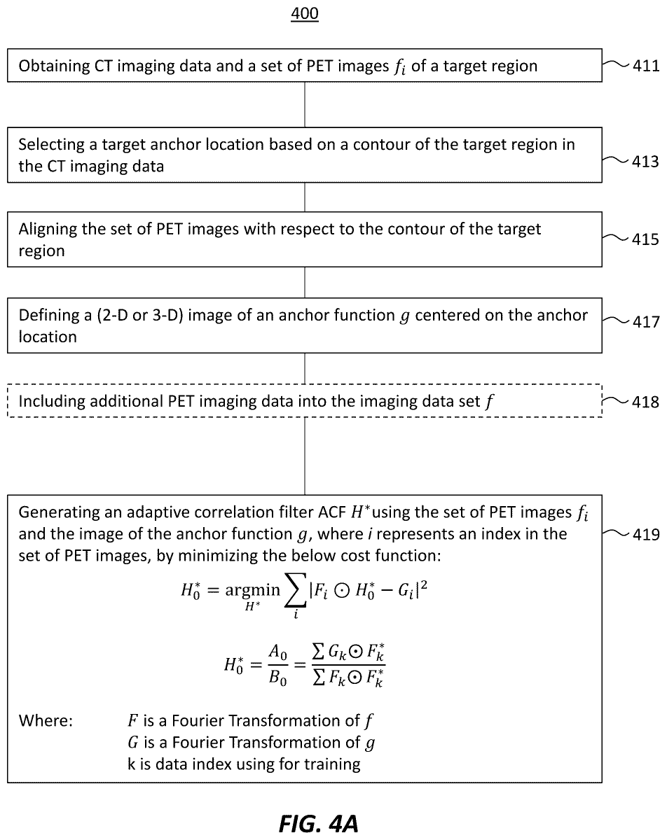

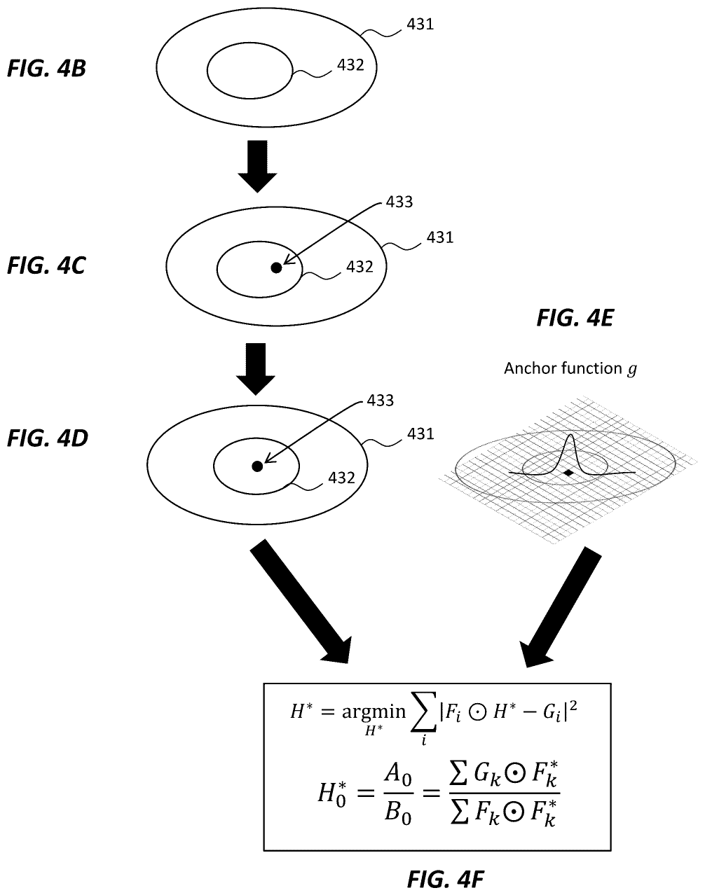

FIG. 4A depicts a flowchart of one variation of a method 400 for generating an ACF h based on planning CT images and planning PET images fᵢ. While the method 400 is described as being performed during BgRT treatment planning (i.e., before a BgRT treatment session), in some variations, method 400 may be performed during a BgRT treatment session, for example, as part of online or real-time treatment adaptation. In some variations, the ACF may be generated using an initial set of PET imaging data acquired just prior to the BgRT treatment session (e.g., PET prescan). Method 400 includes obtaining 411 the CT imaging data and a set of PET images fᵢ of a target region, selecting 413 a target anchor location based on a contour of the target region, aligning 415 the PET images with respect to the contour of the target region, defining 417 an image of an anchor function centered on the anchor location, and generating 419 an ACF using the PET images and the image of the anchor function. In some variations, the obtained CT imaging data may be used to generate one or more CT images, and the target region contour may be defined on the one or more CT images. The CT imaging data may be acquired over a plurality of patient platform positions and in some examples, may be obtained using a diagnostic CT imaging system. The set of PET images may optionally be obtained using a diagnostic PET imaging system and/or the PET detectors on a BgRT PET imaging system.

The contour of the target region may be defined by a clinician, with or without the aid of contouring tools and algorithms (e.g., auto-contouring software). In some variations, the contour of the target region may be a PTV contour, an ITV contour, a BTZ contour, a CTV contour, a GTV contour, and/or the contour or boundary of a PET-avid region. FIG. 4B conceptually depicts a target contour 431 (e.g., a PTV contour in a CT image) and PET imaging data 432 arbitrarily positioned relative to the target contour 431. In some variations, the target anchor location may be a substantially central point of the target region contour. For example, the location of the anchor point may be at a centroid location of the target region. Alternatively, the anchor location may be on a selected anatomical structure that may be coupled to the target region, such as an anatomical structure whose position is associated with the position of the target region. FIG. 4C conceptually depicts selecting 413 a target anchor 433 that is located at a central location of the target contour 431. The target anchor location may be defined or fixed relative to the target contour. FIG. 4D conceptually depicts aligning 415 the PET imaging data 432 to the target contour 431, which may be, in this variation, aligning a central portion of the PET imaging data 432 over the selected target anchor location 433. In some variations, aligning 415 the PET images with respect to the target region contours (on CT images, for example) may include registering the images such that corresponding regions in space are situated in corresponding locations in the image, which may or may not result in concentric alignment of the target contour 431 and the PET imaging data 432. For instance, a selected landmark (e.g., anatomical structure) in the CT image may be aligned with the corresponding landmark in the PET image. In some variations, a region of the PET image having a cluster of high-intensity pixels (e.g., pixels having an intensity value above a threshold value) may be aligned with the target region contour in the CT image, as it is likely that a PET-avid region is a tumor which is to be irradiated.

FIG. 4E conceptually depicts an image of one variation of an anchor function. An anchor function may be a distribution or function that is centered over the target anchor location. In some variations, the anchor function g may be a two-dimensional or three-dimensional Gaussian function having a peak at the target anchor location (herein the two-dimensional Gaussian function is used for a two-dimensional image, and a three-dimensional Gaussian is used when a set of PET images can be used for reconstructing a three-dimensional geometric representation of the target region). Alternatively, the anchor function may be a two-dimensional or three-dimensional delta function centered on the anchor location. Alternatively, or additionally, the cross-correlation image may be an image of a plurality of delta functions where the peaks are in proximity to the anchor location. An image of the target anchor location may comprise a plot or graph of any of the anchor functions described herein and above, centered over the target anchor location. In some variations, the anchor function that is used during treatment planning is the same as the anchor function used to generate an image of the target anchor location during a radiation delivery session.

The method 400 includes an optional step of including 418 additional PET imaging data in the set of PET images fᵢ. The additional PET imaging data may include PET images from another imaging session or other imaging sessions (e.g., diagnostic images), synthetic PET images that simulate variability in PET tracer uptake characteristics, LOR count rate, shifts in the target region location, geometric changes to the target region (e.g., size, shape), background noise levels, or combination thereof. These additional PET imaging may also be aligned with the contour of the target region (e.g., as defined in the CT imaging data). For example, the training set of imaging data may include the day-of PET imaging data (e.g., from the PET prescan prior to irradiation), as well as synthetic or simulated PET imaging data mimicking different tracer uptake conditions, noise levels, location shifts and/or rotations, and the like. For each image in the training set of imaging data, the anchor location is identified so that the ACF can be trained on a variety of imaging characteristics and conditions.

In some variations, as conceptually depicted in FIG. 4F, generating 419 the ACF may comprise iterating through different filter values such that the filter minimizes a sum of squared errors between the cross-correlation of fᵢ and h and the anchor function g when these functions are transformed in the Fourier space. In other words, for H*=[FT(h)]*, the H* is selected such that it minimizes Σᵢ|Fᵢ⊙H*−Gᵢ|². Here, as before, Fᵢ=FT(fᵢ), and Gᵢ=FT(gᵢ). Note that H* for minimizing Σᵢ|Fᵢ⊙H*−Gᵢ|² can be shown to satisfy

$${H}^{*} = {A}_{0} / {B}_{0} = \sum {G}_{i} ⊙ {F}_{i}^{*} / \sum {F}_{i} ⊙ {F}_{i}^{*} .$$

### Here,

$${A}_{0} = \sum {G}_{i} ⊙ {F}_{i}^{*} , and {B}_{0} = \sum {F}_{i} ⊙ {F}_{i}^{*} ,$$

and division operator “/” implies a pointwise operation (similar to pointwise multiplication operator ⊙).

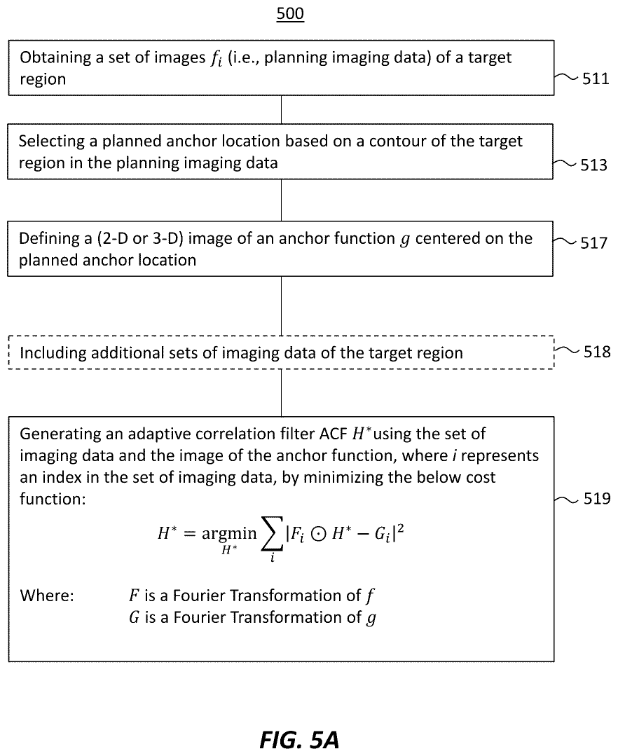

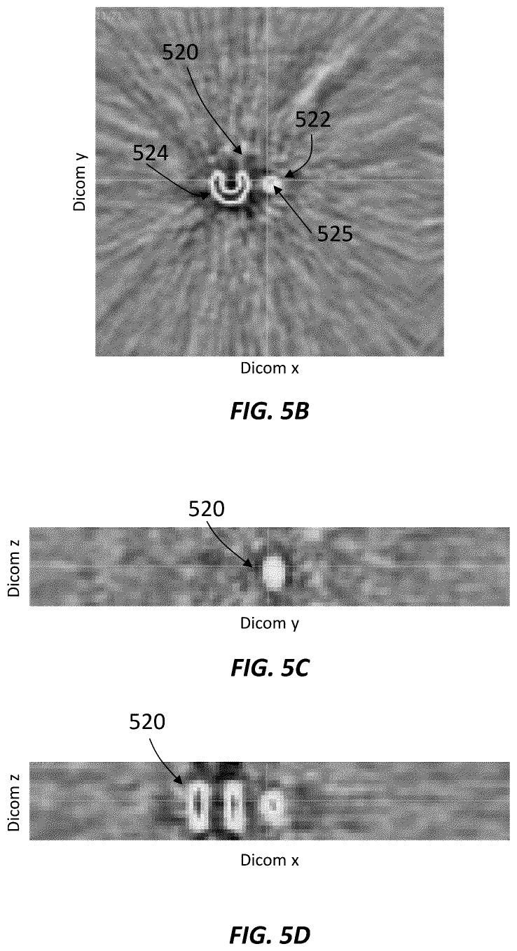

FIG. 5A depicts a flowchart of one variation of a method 500 for generating an ACF h for image-guided radiation therapy (e.g., IMRT, SBRT, SRS) based on planning images (e.g., CT images). In some variations, image-guided radiation therapy includes only one imaging modality, for example, only CT imaging data, only MR imaging data, or only X-ray imaging data. In some variations, a BgRT workflow may include performing method 500 during treatment planning and/or during a BgRT treatment session.

The method 500 includes obtaining 511 a set of planning images fᵢ of a target region, selecting 513 a planned target anchor location based on a contour of the target region, defining 517 an anchor function g centered at a planned target anchor location, and generating 519 an ACF using the set of planning images fᵢ and the anchor function g. The planning images may be acquired over a plurality of patient platform positions. In some variations, the planning images may include CT imaging data. In various implementations, step 519 of method 500 may be similar or the same as step 419 of method 400. For example, the ACF h may be configured to be a filter that minimizes a sum of squared errors between the cross-correlation of fᵢ and h and the anchor function g when these functions are transformed in the Fourier space. In other words, filter H* is calculated or selected such that it minimizes Σᵢ|Fᵢ⊙H*−Gᵢ|² as described in relation to step 419.

In some variations, the target anchor location may be a substantially central point of the target region contour. For example, the location of the anchor point may be at a centroid location of the target region. Alternatively, the anchor location may be on a selected anatomical structure that may be coupled to the target region, such as an anatomical structure whose position is associated with the position of the target region, which may or may not be at a centroid location of the target region.

Further, the method 500 may include defining 517 an anchor function g centered at a planned target anchor location (e.g., similar to a step of 417 of method 400). For example, the anchor function g may be a two-dimensional or three-dimensional Gaussian function having a peak at the target anchor location (herein the two-dimensional Gaussian function is used for a two-dimensional image, and a three-dimensional Gaussian is used when a set of images can be used for reconstructing a three-dimensional geometric representation of the target region). Alternatively, the anchor function may be a two-dimensional or three-dimensional delta function centered on the anchor location. Alternatively, or additionally, the cross-correlation image may be an image of a plurality of delta functions where the peaks are in proximity to the anchor location. In some variations, the anchor function that is used during treatment planning is the same as the anchor function used to generate an image of the target anchor location during a radiation delivery session.

The method 500 may include an optional step of including 518 additional CT imaging data to form one or more CT images that can be included into the set of images fᵢ. The additional CT imaging data may be aligned or registered with the original planning image (e.g., the image in step 511). The additional CT imaging data may include variabilities, such as detector inhomogeneities, scatter noise, shifts in the target region location, geometric changes to the target region (e.g., size, shape) and/or may be from other one or more imaging sessions (e.g., diagnostic images). For example, the training set of imaging data may include the day-of CT imaging data (e.g., from the localization CT prior to irradiation), as well as synthetic or simulated CT imaging data mimicking different patient positions, noise levels, location shifts and/or rotations, and the like. For each image in the training set of imaging data, the anchor location is identified so that the ACF can be trained on a variety of imaging characteristics and conditions.

FIGS. 5B-5D depict spatial domain images of an ACF generated in a simulation conducted using experimentally acquired imaging data using the method 400. In the spatial domain, the ACF 520 is depicted (i.e., represented) as a template. The ACF template 520 was generated using PET imaging data experimentally acquired on a BgRT radiotherapy system of a phantom having a PET-avid target region 522 and a PET-avid organ at risk (OAR) 524. In this experiment, the PET-avid target region 522 is shaped as a sphere, and is adjacent to the OAR 524, which has a C-shape in the axial view. FIG. 5B is an axial view of the ACF template 520, FIG. 5C is a sagittal view of the ACF template 520, and FIG. 5D is a coronal view of the ACF template 520. For this ACF template, the target anchor location was selected to be at the point 525 depicted in FIG. 5B, which is in the center of the PET-avid target region 522. During a treatment session, the ACF template 520 may be applied (e.g., cross-correlated) with newly-acquired imaging data to generate a cross-correlation or filtered image. Then, the updated location of the target anchor may be determined from the cross-correlation image and used to calculate (e.g., update) the radiation fluence for delivery. While this ACF template 520 was calculated using PET imaging data, it should be understood that the ACF template may be calculated using any type of imaging data, including, but not limited to, X-ray, CT, ultrasound, and/or MR imaging data. Moreover, in this example, the ACF template was calculated based on the contours of the OAR and the target region, which is why the ACF template includes both the OAR and the target region. However, in other variations, the ACF template may be calculated based on the contour of the target region and not on the contour(s) of the OAR(s). In other variations, the ACF template may be calculated based on only the contour of the OAR(s). For example, in BgRT, a biology-tracking zone (BTZ) may be defined for the set of target region images. A BTZ may be a region that encompasses the range of motion of the target region or PTV, but excludes the range of motion of an OAR. The contours of the BTZ may be the boundaries of a spatial filter used to select the PET imaging data that is to be used for guiding radiation delivery. The BTZ spatial filter may be applied to the imaging data before an ACF template is cross-correlated with the imaging data. In this variation, the ACF template may be defined based on the target region contour (but not the OAR contour), since the OAR region is filtered out by the BTZ. Optionally, some methods may comprise generating a graphical representation comprising the boundaries of the BTZ and a point that represents the current/updated location of the target anchor location.

In some variations, the ACF that is generated during treatment planning may be updated (e.g., trained, adapted) with additional imaging data. In the case of BgRT, the ACF may be updated using PET prescan imaging data acquired at the beginning of a treatment session. Optionally, the cumulative PET imaging data as part of BgRT radiation delivery may be used to generate a treatment time PET image and the treatment time PET image may be used to further update the ACF (e.g., for the next treatment session). In some variations, the quality of the treatment time PET image may be evaluated before it is used to update the ACF. That is, in order to use a treatment time PET image to update the ACF, the regions of the PET image having the highest intensity levels may need to have substantial overlap and/or alignment with the target region contour on a planning and/or localization CT. Alternatively, or additionally, a confidence metric may be used to evaluate whether an image (e.g., filtered image, cross-correlation image) may be used to update the ACF. For example, a confidence metric value of an image may be calculated and if its value is above a threshold value, the image may be used to update the ACF.

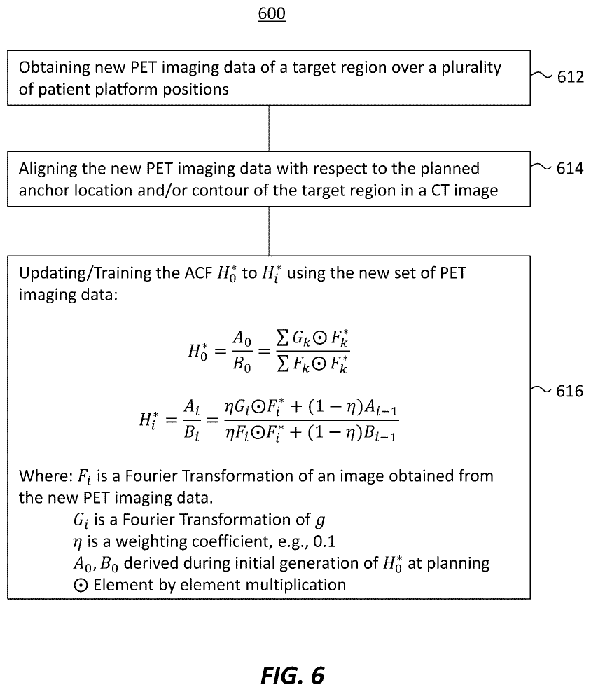

FIG. 6 shows one variation of a method 600 for updating the ACF h based on PET imaging data acquired during a BgRT treatment session. The method 600 includes obtaining 612 new PET imaging data, aligning 614 the new PET imaging data with respect to a planned anchor location and/or contour of the target region in a CT image, and updating 616 the ACF h using a new set of PET imaging data. The new PET imaging data may be acquired over various patient platform positions. In some cases, the new PET imaging data may be obtained during a prescan procedure (e.g., the PET images may be obtained before starting the BgRT treatment session). In some cases, the new PET imaging data may be obtained using a diagnostic PET imaging system.

In some variations, aligning 614 a PET image with respect to the target region contours (on CT images, for example) may include selecting a landmark (e.g., anatomical structure) in the PET image and aligning the selected landmark with the corresponding landmark in the CT image. Various alignment approaches, as previously described in relation to step 415 of method 400 may be used for the aligning step 614.

In some variations the updating 616 of the ACF h may include updating H* (herein, as before, H*=[FT(h)]*). The H* may be updated iteratively based on an additional PET image fᵢ. For example, if H*ᵢ₋₁ is a complex conjugate of a Fourier transform of the ACF h before the update, the complex conjugate of the updated Fourier transform is denoted as H*ᵢ and may be given by

$${H}_{i}^{*} = {A}_{i} / {B}_{i} , {A}_{i} = η {G}_{i} ⊙ {F}_{i}^{*} + ( 1 - η ) {A}_{i - 1} , and {B}_{i} = η {F}_{i} ⊙ {F}_{i}^{*} + ( 1 - η ) {B}_{i - 1} , and {A}_{0} = {\sum }_{1}^{{k}_{m}} {G}_{k} ⊙ {F}_{k}^{*} , {B}_{0} = {\sum }_{1}^{{k}_{m}} {F}_{k} ⊙ {F}_{k}^{*} .$$

Herein, as described above, Gᵢ=G_{i,uv} and Fᵢ=F_{i,uv} are two dimensional matrices (for two dimensional images), and operator ⊙ implies elementwise multiplication

$${G}_{i} ⊙ {F}_{i}^{*} = {G}_{i , uv} \cdot {F}_{i , uv}^{*} .$$

It should be noted that in some cases, Gᵢ and Fᵢ may be three-dimensional images matrices Gᵢ=G_{i,uvw} and F_{i,uvw} corresponding to a volumetric representation of the PET image fᵢ and operator ⊙ implies elementwise multiplication

$${G}_{i} ⊙ {F}_{i}^{*} = {G}_{i , uvw} \cdot {F}_{i , uvw}^{*} .$$

Herein, and elsewhere, as described before Gᵢ=FT(gᵢ), and Fᵢ=FT(fᵢ). Further, parameter η describes contribution (e.g., weight) of the new PET image fᵢ when updating Hᵢ. For example, when parameter η=0, no update is performed, and when parameter η=1, only new PET image fᵢ is considered when calculating updated Hᵢ. In general n may be a relatively small number (e.g., η may be in the range of about 0.01 to about 0.2). In some cases, η˜0.1. Once Hᵢ is computed, the updated ACF hᵢ can be computed as hᵢ=FT⁻¹(Hᵢ). Further, note that images indexed k=1 to kₘ (e.g., images represented by Fₖ) and functions Gₖ correspond respectively to a training set of images and delta functions for generating an initial estimate for the ACF h (herein, kₘ is the number of images).

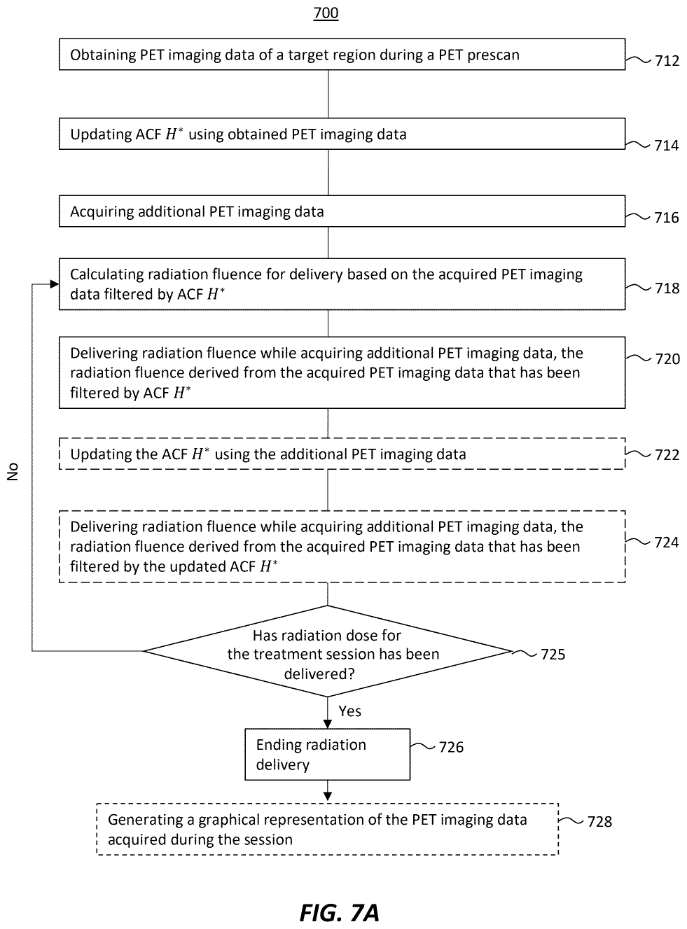

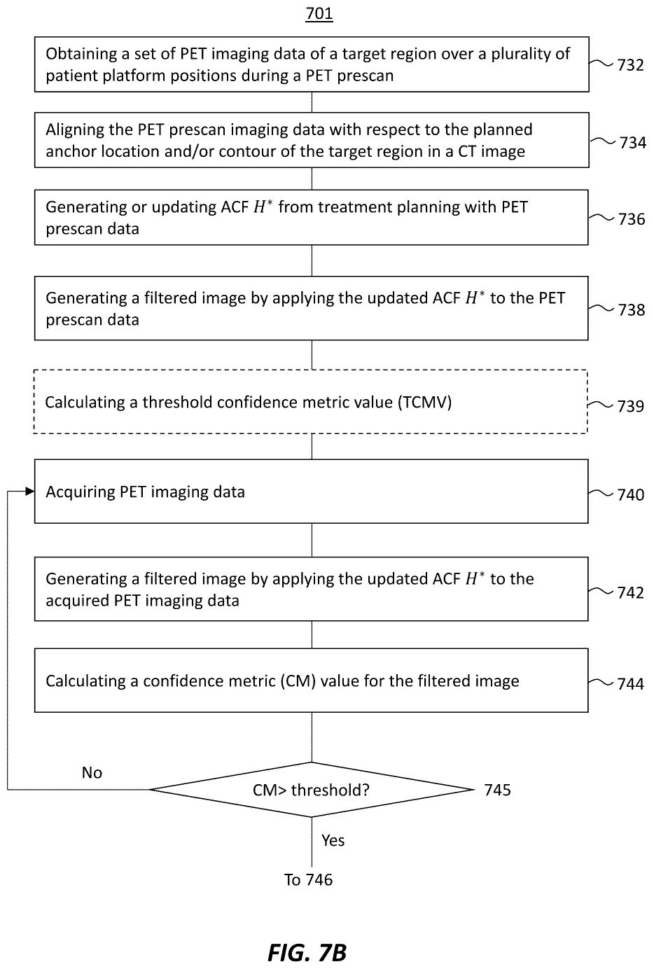

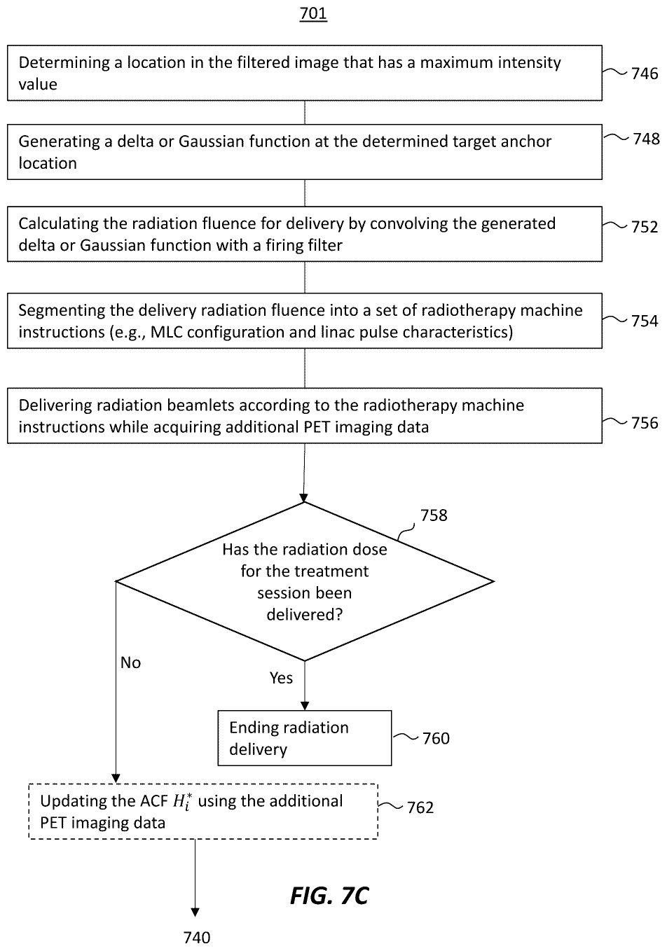

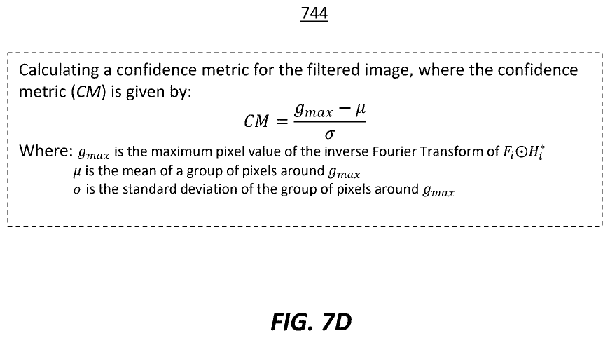

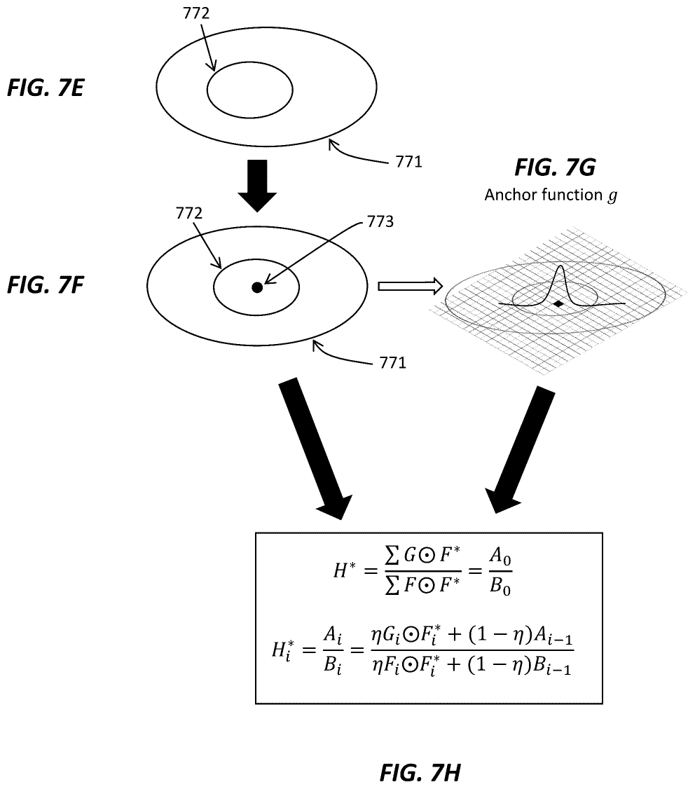

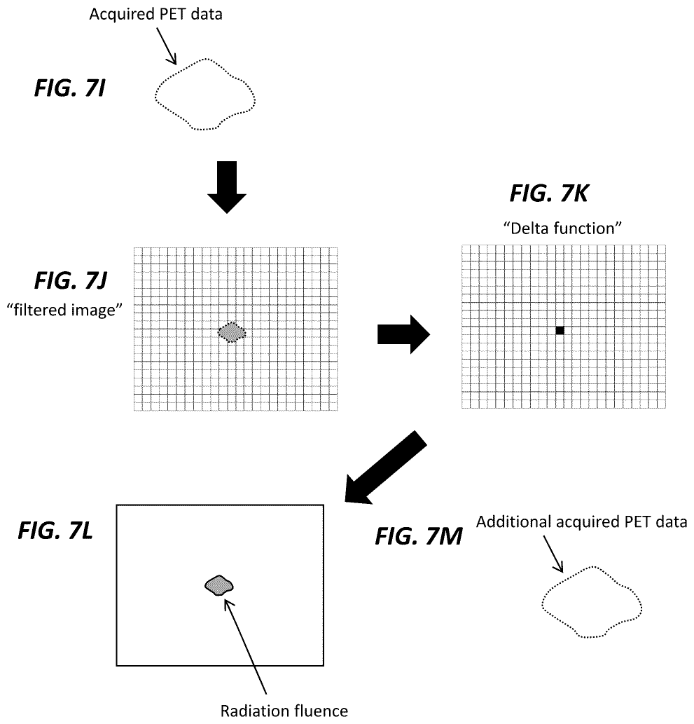

FIG. 7A depicts one variation of a method of updating an ACF h during a BgRT treatment session based on PET imaging data acquired during the BgRT treatment session, as well as using the updated ACF h to deliver radiation fluence.

The method 700 includes obtaining 712 PET imaging data of a target region (e.g., a PTV) during a PET prescan, updating 714 the ACF h (the updating 714 may be the same or similar to the method 600 as described above in relation to FIG. 6), acquiring 716 additional PET imaging data, calculating 718 radiation fluence for delivery based on the acquired PET imaging data filtered (e.g., cross-correlated) by the ACF (such filtering may be the same or similar to the filtering described in methods 300, 301), and delivering 720 radiation fluence while acquiring additional PET imaging data. Method 700 may optionally include updating 722 the ACF using the acquired additional PET imaging data (the updating 722 may be similar to or the same as the method 600 described in relation to FIG. 6), and delivering 724 radiation fluence while acquiring additional PET imaging data. The radiation fluence for delivery may be derived from the acquired PET imaging data filtered or cross-correlated by the updated ACF, using any of the methods described above (e.g., the methods 300, 301). Method 700 may include determining 725 whether the radiation dose for the treatment session has been delivered, and ending 726 the radiation dose when the prescribed radiation dose has been delivered. Alternatively, when additional radiation needs to be delivered (i.e., the prescribed radiation dose has not yet been delivered), method 700 may comprise returning to step 718 to continue with radiation delivery.

The PET prescan may be performed using a BgRT PET imaging system. For example, the PET prescan procedure may be performed at the start of the BgRT treatment session, which may help ensure that there is sufficient PET tracer uptake and signal strength to guide radiation delivery. In various cases, the ACF h may be initially determined using a diagnostic PET imaging data obtained using a diagnostic PET imaging system and/or using a BGRT PET imaging system (e.g., the ACF h may be determined based on a set of PET images using, for example, method 400, as described above in relation to FIG. 4A).

The radiation fluence may be calculated in step 718 of method 700 using various approaches (e.g., using approaches as described above, as related to step 318 of method 300). For instance, calculating the radiation fluence may include applying a firing filter to the filtered (or cross-correlation) image, which may be determined by applying the ACF h to a PET image obtained from the PET imaging data. Optionally, the PET imaging data may be spatially filtered by the BTZ before the ACF is applied. In some variations, as described in step 318 of method 300, calculating the radiation fluence includes shifting a planned radiation fluence according to the target anchor location. Alternatively, or additionally, calculating the radiation fluence may comprise determining the target anchor location from the cross-correlation or filtered PET imaging data, generating an image of the target anchor location (e.g., by generating image of a Gaussian or delta function centered over the target anchor location, where the Gaussian or delta function is the same function that was used during treatment planning), and convolving the image of the target region location with the BgRT firing filters. This is one method that may be used to convert the filtered (or cross-correlation) image into a radiation fluence. Alternatively, or additionally, the target anchor location can be used to shift the planned radiation fluence relative to a PET-avid OAR to avoid or reduce irradiation of the OAR and still continue delivery radiation to the tumor.

In some variations, calculating or determining the radiation fluence for delivery may optionally comprise determining whether the determined target anchor location (and/or an OAR anchor location determined in the same way the target anchor location is determined) deviate too much from its expected anchor location. The expected anchor location may be the location of the anchor defined during planning, and/or may be the location of the anchor determined based on initial imaging data acquired during an earlier portion of the treatment session (e.g., PET or CT prescan). If the determined target anchor location (and/or the determined OAR anchor location) is greater than a pre-defined distance from its expected location, the radiotherapy system may generate a notification to the user and provide an option to gate or pause radiation delivery. In some variations, the pre-defined distance may be determined from a global system configuration that bounds the maximum shift allowed, or determined by a clinician at the time of treatment planning to optimize both safety and treatment efficiency.

The delivery 720 of the radiation fluence (e.g., the calculated radiation fluence, as calculated in step 718) may be performed while acquiring additional PET imaging data. Alternatively, the delivery 720 of the radiation fluence may be performed without acquiring additional PET imaging data. The radiation fluence may be derived from the acquired PET imaging data that has been filtered by ACF H* using any of the methods described herein (e.g., methods described above and depicted in FIGS. 3A-3B). Optionally, this additional PET imaging data may be used for updating 722 the ACF h. Method 700 may comprise continuing to deliver 724 radiation fluence calculated based on the updated ACF.

As described above, method 700 may include evaluating 725 whether the radiation dose for the treatment session has been delivered. If the radiation dose has been delivered (step 725, Yes), the method 700 includes ending 726 the treatment session. Alternatively, if the radiation dose has not been delivered (step 725, No), the method 700 includes returning to step 718.

In some variations, the method 700 optionally includes generating 728 a graphical representation of the PET imaging data (e.g., generating the graphical representation on a display associates with the BgRT imaging system) acquired during the BgRT treatment session. For example, the graphical representation may be one or more PET images of a target region obtained during the BgRT treatment session. The one or more PET images may be reviewed by a medical professional (e.g., a medical professional may select a particular one of the PET images to be displayed on the display associated with the BgRT imaging system). Alternatively, or additionally, the graphical representation may comprise the BTZ contour and a labeled point (or points) of the target anchor location(s) within the BTZ contour. For example, if the target region is moving during the treatment session and the target anchor location is updated for each shuttle pass, the graphical representation may comprise the BTZ contour and a point (or any discrete graphic symbol) for each of the calculated target anchor locations for each shuttle pass. Optionally, some methods may comprise calculating the difference between the current target anchor location and a previous target anchor location, and generating a graphical representation that includes that difference and an indicator as to whether that difference is in an acceptable range. Optionally, some methods may define the previous target location as the first target location at the start of the treatment session. Optionally, the previous target location can be updated based on a specified time window. In some variations, the previous target location could be the first point in the time window, the last point in the time window, or the average of the previous target locations in the time window. In some variations, the time window may be a global system configuration or determined by a clinician at the time of treatment planning. If the location difference is greater than an acceptable range, the graphical representation may include a visual indicator (e.g., color, flashing animation, pop-up window, etc.) and may optionally also generate an audible notification. The operator may decide whether to pause radiation or continue. Alternatively, or additionally, if the location difference is greater than acceptable range, the radiation delivery may be automatically gated or paused. In some variations, the acceptable range may be a global system configuration or determined by a clinician at the time of treatment planning. Further, in some optional implementations, the BgRT system may include a suitable interface for an electronic device (e.g., the electronic device may be a computer such as a desktop or a laptop, a tablet, a smartphone, and the like, and the suitable interface may be a display, a keyboard, a mouse, a touchpad, and the like) that can be used by a medical professional to review other data associated with PET images. For example, a BgRT system may be configured to allow the medical professional to review the ACF h, the form (e.g., size and shape) of a Gaussian g; for a PET image fᵢ, any one of the PET images fᵢ, a difference between any pair of images selected from PET images fᵢ, filtered images (or cross-correlated images) obtained by applying ACF h to any one of the PET images fᵢ, a difference between filtered images, and the like.

FIG. 7B depicts one variation of a method 701 of using an ACF during a BgRT treatment and updating the ACF h during based on at least some of the PET imaging data acquired during the treatment session, as well as using the updated ACF h to calculate the radiation fluence for delivery.

The method 701 includes obtaining 732 PET imaging data of a target region (e.g., a PTV, BTZ) over a plurality of patient platform positions during a PET prescan. The step 732 may be similar to the step 712 of method 700. Further, method 701 includes aligning 734 the PET prescan imaging data with respect to the planned anchor location and/or contour of the target region in a CT image. Further, the method 701 may include generating or updating 736 an ACF h for treatment planning based on the obtained PET imaging data as follows:

$${H}_{0}^{*} = \frac{\sum G ⊙ {F}^{*}}{\sum F ⊙ {F}^{*}} = \frac{{A}_{0}}{{B}_{0}} \\ {H}_{i}^{*} = \frac{{A}_{i}}{{B}_{i}} = \frac{η {G}_{i} ⊙ {F}_{i}^{*} + ( 1 - η ) {A}_{i - 1}}{η {F}_{i} ⊙ {F}_{i}^{*} + ( 1 - η ) {B}_{i - 1}} .$$

Where: Fᵢ is a Fourier Transformation of the PET prescan imaging data, Gᵢ is a Fourier Transformation of the planned anchor location function, and n is a weighting coefficient, e.g., 0.1.

The method 701 further includes generating 738 a filtered or cross-correlation image by applying the updated ACF h to the PET prescan imaging data.

The method 701 may further include an optional step 739 of calculating a threshold confidence metric value (TCMV) for the filtered image obtained in the step 738. The CM value may be calculated as shown in FIG. 7D as a ratio of a difference between a maximum value gₘₐₓ of g=FT⁻¹(Fᵢ⊙H*ᵢ) of the filtered image and a mean value μ of a group of pixels selected about gₘₐₓ, and a standard deviation σ of the group of pixels around gₘₐₓ. In one implementation, the group of pixels of the filtered image about gₘₐₓ may be all the pixels with values above a value of about half gₘₐₓ or at about 60%, 70%, 80%, 90%, or 95% of gₘₐₓ. The TCMV may be used to evaluate whether later-acquired images are similar enough to an image acquired at the start of a treatment session. Images that deviate substantially from the image acquired at the start of the treatment session may be assigned a low confidence metric value because they may indicate errors in the image acquisition, equipment malfunction, and/or patient changes (e.g., tumor and/or patient motion) that are large enough such that delivering radiation based on the later-acquired images would be unsafe.

Further, the method 701 may further include acquiring additional 740 PET imaging data and generating 742 a filtered image by applying the ACF h to the acquired additional PET imaging data. The step 742 may be the similar to the step 738, with the difference that the updated ACF h is applied not to the prescan PET imaging data but to the acquired additional PET imaging data.

Further, the method 701 includes calculating a confidence metric (CM) value for the filtered image obtained in the step 744, for example by using the method depicted and described in FIG. 7D. If the CM value is above a threshold value (step 745, Yes), the method 701 proceeds to determining 746 a target anchor location by identifying the region in the filtered (or cross-correlation) image that has a maximum pixel or voxel intensity value. A high intensity value in any of the filtered or cross-correlation images described herein may be understood to be a region in the acquired imaging data that has an elevated degree of correlation with the initial template (i.e., ACF template). In one variation, the threshold value may be selected by a medical professional, or, when the optional step 739 is performed, the threshold value may correspond to the TCMV. Alternatively, if the CM value is below or equal to the threshold value (step 745, No), the method 701 proceeds to step 740 and new PET imaging data is acquired.

After completion of step 746, method 701 may comprise generating 748 a delta or Gaussian function at the target anchor location, i.e., an image of the target anchor location. In an example implementation the delta function is determined numerically to have a value of one at the target anchor location and value of zero at any other location. When Gaussian function is used, the Gaussian function is selected to have a maximum value at the target anchor location. Alternatively, or additionally, generating 748 a delta function (or Gaussian function) may comprise generating a plurality of delta functions (or Gaussian functions) where the peaks are in proximity to the target anchor location. While the examples provided herein uses a delta function or a Gaussian function, it should be understood that one or more other functions may be applied to the target anchor location, for example, a triangular function, or any similar function (e.g., any square integrable function such as a box function). An image of the function based on the target anchor location may be used to calculate a fluence for delivery. In some variations, the anchor function that is used to generate an image of the target anchor location during a radiation delivery session is the same as the anchor function used in treatment planning.

For example, the method 701 includes calculating 752 the radiation fluence by applying a firing filter to the target anchor location, where the firing filter is calculated based on previously-acquired imaging data of the target region (e.g., calculated during treatment planning based on an image of the target anchor location defined using treatment planning images). In one implementation, applying the firing filter to the target anchor location includes convolving an image of the target anchor location with the firing filter. Herein, the image of the target anchor location may be generated by placing a Gaussian (or any other distribution function such as a delta function, etc.) over the location of the target anchor. In some variations, the anchor function that is used to generate an image of the target anchor location during the radiation delivery session is the same as the anchor function used in treatment planning. The location of the target anchor may be determined from a filtered or cross-correlation image using any of the methods described herein. For example, the filtered or cross-correlation image may be obtained by cross-correlating the ACF h with a particular one of PET images of the target region, and the location of the target anchor may be the region of the cross-correlation image having pixels and/or voxels above a certain intensity threshold.

In one implementation, the method 701 may include calculating 752 the radiation fluence for delivery by convolving the generated delta function or Gaussian function with a firing filter. As described above, firing filters are a mapping function calculated during treatment planning that transform imaging data into a corresponding radiation fluence. The distribution function used to generate the image of the target anchor location may be the same distribution function used to generate the ACF during treatment planning. For example, if the image of the anchor location used during treatment planning to calculate the firing filters and/or ACF is a Gaussian function centered over the anchor location, the image of the target anchor location during radiation delivery may also be the same Gaussian function (e.g., having the same height and/or RMS width or “spread”) centered over the updated target anchor location.

In various implementations, the method 701 includes segmenting 754 the delivery radiation fluence into a set of radiotherapy machine instructions (e.g., MLC configuration and linac pulse characteristics) and delivering 756 radiation beamlets according to the radiotherapy machine instructions while acquiring additional PET imaging data. Segmenting 754 the calculated radiation fluence may include dividing the radiation fluence into portions of radiation (e.g., beamlets having a certain spatial extent, location, and intensity) deliverable at a firing position of the therapeutic radiation source, and generating the machine instructions that would shape the radiation beam to attain the deliverable portions of radiation at each firing position. In some variations, the radiotherapy machine instructions may also comprise instructions that specify beam station positions of the patient platform during radiation delivery.

In some variations, the method may include evaluating 758 whether the radiation dose for the treatment session has been delivered. If the radiation dose has been delivered (step 758, Yes) the method 701 may include ending 760 the treatment session. Alternatively, if the radiation dose has not been delivered (step 758, No), the method 701 may optionally include updating the ACF h using the additional PET imaging data acquired during radiation fluence delivery. Further, the method 701 includes returning to the step 740 to acquire new PET imaging data to guide radiation delivery until the prescribed dose for the treatment session has been delivered.

FIGS. 7E-7H conceptually depict one variation of a method of updating or training an ACF with additional imaging data (e.g., described above in methods 700 and 701). For example, FIG. 7E conceptually depicts obtaining PET imaging data of a target region during a PET prescan (i.e., obtaining imaging data 712, 732 in methods 700, 701). The PET imaging data 772 may be overlaid with the target region contour 771, which may be delineated on a CT image (or a PET/CT image). The contour of the target region may be any of the contours described herein (e.g., PTV, ITV, BTZ, CTV, GTV). FIG. 7F conceptually depicts aligning the PET imaging data 772 to the target contour 771 (e.g., step 734, of method 701), which may be, in this variation, aligning a central portion of the PET imaging data 772 over the target anchor location 773 that corresponds with the target contour 771 (as defined during treatment planning). In some variations, the target anchor location in the PET imaging 772 data may be determined using any of the methods described herein, and the newly determined location of the target anchor may be aligned with the target anchor location from treatment planning. FIG. 7G conceptually depicts an image of the target anchor location 773, which is an anchor function (e.g., a numerical delta or Gaussian function as described above in step 748 of method 701) that is centered over the target anchor location. FIG. 7H conceptually depicts updating the ACF h (e.g., the step 714 of method 700 or step 736 of method 701) by updating a Fourier transform H=FT(h). The updated ACF h may then be applied to imaging data acquired thereafter. For example, FIG. 7I is a conceptual representation of additionally acquired PET imaging data (e.g., step 740 of method 701). The ACF h calculated/updated above may be applied to the newly acquired imaging data to generate a filtered image as depicted in FIG. 7J (e.g., corresponding to step 742 of method 701). The target anchor location may be determined from the filtered image of FIG. 7J and an image of the target anchor location may be generated, for example, of a numerical delta function centered over the target anchor location as depicted in FIG. 7K (e.g., corresponding to step 748 of method 701). FIG. 7L is a conceptual depiction of a radiation fluence for delivery calculated based on the image of the image of the target anchor location of FIG. 7K. For example, the radiation fluence may be generated by convolving the image of the target anchor region with firing filters, and/or may be generated by shifting the planned fluence map to align with the updated location of the target anchor (e.g., steps 752-756). During the delivery of the radiation fluence of FIG. 7L, additional PET imaging data may be acquired (concurrently or sequentially with radiation delivery), as conceptually depicted in FIG. 7M (e.g., step 756 of method 701).

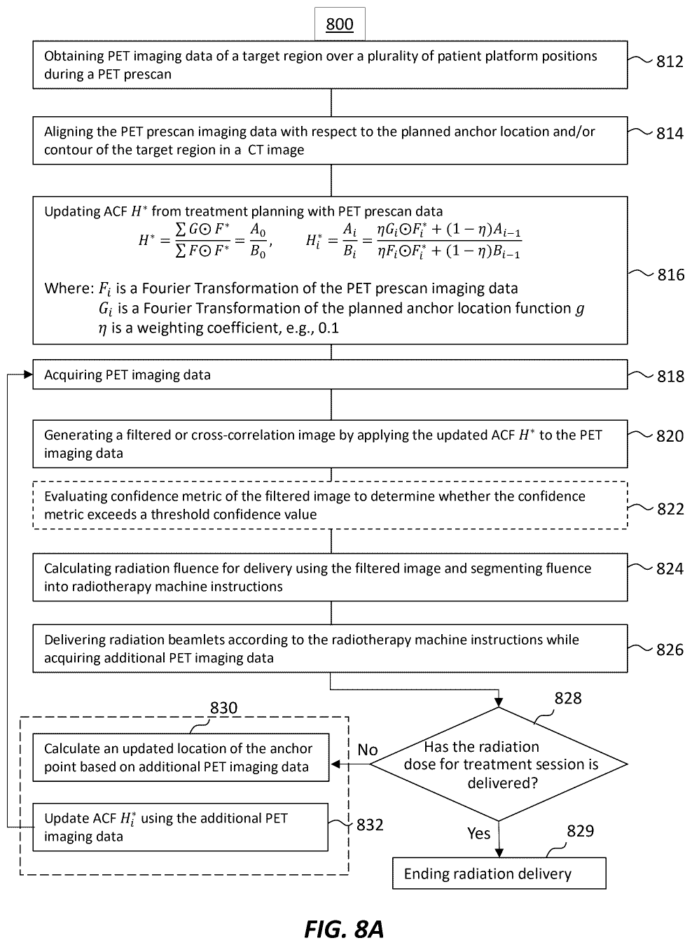

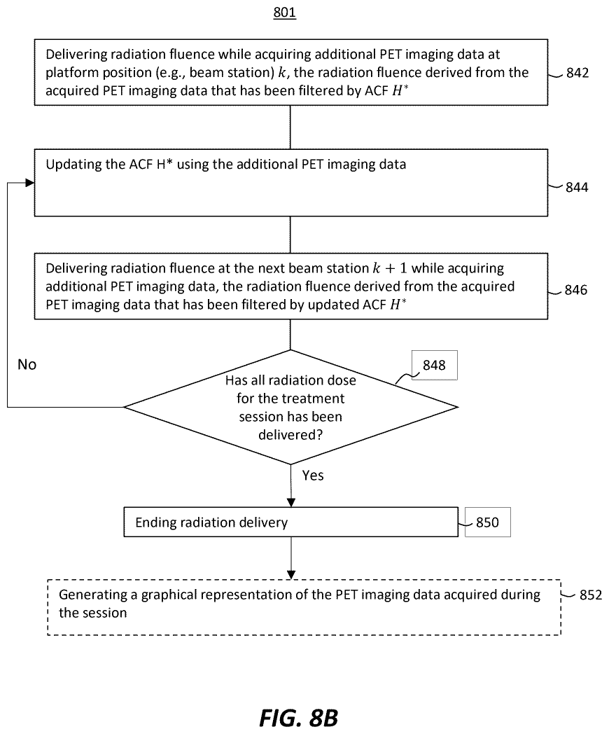

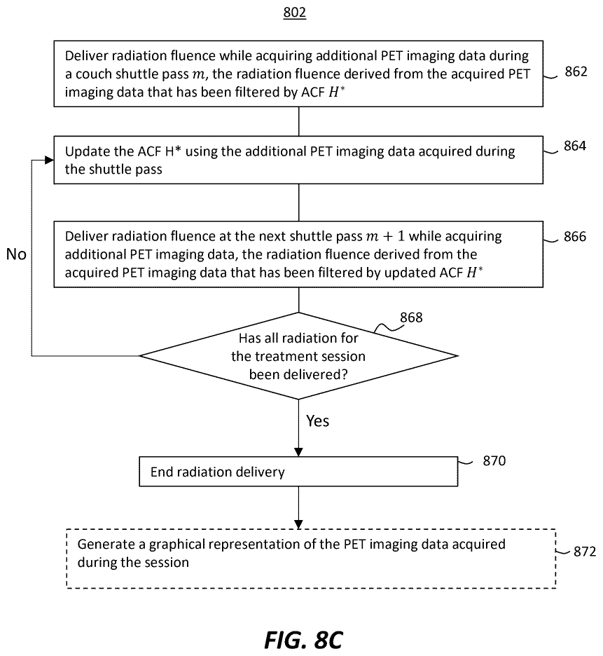

Optionally, in some variations, a target anchor location may be updated during a BgRT treatment session. In various cases, during the BgRT treatment session, the patient platform may be placed into a plurality of positions or beam stations. An example method 800 of updating the target anchor location is shown in FIG. 8A. The method 800 includes obtaining 812 PET imaging data of a target region over a plurality of patient platform positions during a PET prescan procedure. The PET prescan procedure may be performed at the beginning of a BgRT treatment session.

Further, the method 800 may include aligning 814 the PET prescan imaging data with respect to a planned target anchor location and/or CT image contour of the target region in one or more CT images. The planned target anchor location may be determined based on the one or more CT images (e.g., as described above the planned target anchor location may be determined as a centroid of the contour, as a point on the contour, and/or may be defined based on the relative position to the target contour during treatment planning).

The ACF may optionally be updated (i.e., trained) with the imaging data acquired during the PET prescan procedure. For example, the method 800 may include updating 816 the ACF h. The updating step 816 may be similar to or the same as the updating step 616 of method 600. The updated ACF may be used to calculate the radiation fluence for the treatment session. In one variation, method 800 may comprise acquiring 818 additional PET imaging data, and generating 820 a filtered or cross-correlation image by applying the updated ACF h to the acquired additional PET imaging data (step 820, for example, may be similar to or the same as step 742 of the method 701). After completion of the step 820, the method 800 may include an optional step of evaluating 822 a confidence metric (CM) of the filtered image to determine whether the confidence metric exceeds a threshold confidence value (similar to the confidence metric calculations described for method 701, e.g., as described in FIG. 7D).

The method 800 may include calculating 824 radiation fluence for delivery using the cross-correlation image. In one variation, calculating 824 the radiation fluence for delivery may comprise determining the location of the target anchor in the cross-correlation image, generating an image of the target anchor location, and convolving the image of the target anchor location with a firing filter. In some variations, calculating 824 the radiation fluence for delivery may comprise determining the location of the target anchor in the cross-correlation image, and shifting the planned fluence map to align with the current location of the target anchor (e.g., to best match the relative positions of the fluence map and the target anchor location as defined during treatment planning). Alternatively, calculating 824 the radiation fluence for delivery may comprise convolving an image of the target anchor location (i.e., derived from the cross-correlation image) with a firing filter. In some variations, calculating 824 the radiation fluence for delivery may comprise convolving the cross-correlation image with a firing filter. The calculated radiation fluence may then be segmented into radiotherapy machine instructions. Following step 824, the method 800 includes delivering 826 radiation beamlets according to the radiotherapy machine instructions while acquiring additional PET imaging data. Further, the method 800 includes evaluating 828 whether the planned radiation dose for the treatment session has been delivered. If the planned radiation dose has been delivered (step 828, Yes) the method 800 proceeds to ending 829 the treatment session. Alternatively, if the radiation dose has not been delivered (step 828, No), the method 800 may optionally comprise updating 830 the location of the target anchor based on the additional acquired PET imaging data (e.g., the PET imaging data acquired at the step 826). The location of the target anchor may have moved due to motion of the target region and/or the patient. Further, the method 800 may optionally include updating 832 the ACF h using the additional PET imaging data (e.g., using the method depicted in FIG. 6). Further, the method 800 includes returning to the step 818 to acquire new PET imaging data to guide further radiation delivery. For any of the methods described herein, the ACF may be updated at any point prior to, or during, a radiation delivery session. For example, the ACF may be updated after radiation is delivered at a particular beam station (i.e., patient platform position), before radiation is delivered at a beam station, at the end of a shuttle pass, at the beginning of a shuttle pass, and/or any time when there is additional imaging data of the target region that may help improve the accuracy of the ACF template to identify a target region within an image.

FIG. 8B depicts a flowchart of one variation of a method 801 for updating ACF h during a BgRT treatment session and delivering radiation fluence, where the update and the radiation fluence delivery can be performed at each beam station (e.g., patient platform position). The method 801 includes delivering 842 radiation fluence while acquiring additional PET imaging data at a platform position pₖ (e.g., at a beam station, herein the index k is used to refer to different platform positions). In some variations, the radiation fluence may be derived based on the acquired PET imaging data that has been filtered by the ACF h. Further, the method 801 may include updating 844 the ACF h based on the acquired additional PET imaging data in step 842. In one variation, the ACF may be updated using the cumulative PET imaging data acquired at a beam station. Following the step 844, the method 801 includes delivering 846 radiation fluence at the next platform position pₖ₊₁ (e.g., at a next beam station k+1) while acquiring additional PET imaging data. Similar to the step 842, the radiation fluence at step 846 may be derived based on the acquired PET imaging data that has been filtered by the updated ACF h. Further, the method 801 includes evaluating 848 whether the planned radiation dose for the treatment session has been delivered. If the radiation dose has been delivered (step 848, Yes) the method 801 proceeds to ending 850 the treatment session. Alternatively, if the planned radiation dose has not been delivered (step 848, No), the method 801 may optionally comprise updating 844 the ACF h based on the additional acquired PET imaging data (e.g., the PET imaging data acquired at the step 842 or step 846) and continuing radiation delivery based the additional acquired PET imaging data.

In some cases, the method 801 optionally includes generating 852 a graphical representation of the PET imaging data (e.g., generating the graphical representation on a display associated with the BgRT imaging system) acquired during the BgRT treatment session. The step 852 may be similar to or the same as step 728 of method 700, as described above in relation to FIG. 7A.

FIG. 8C shows a method 802 for updating the ACF h during a BgRT treatment session and delivering radiation fluence, where the update and the radiation fluence delivery can be performed at each couch shuttle pass. The method 802 may include delivering 862 radiation fluence while acquiring additional PET imaging data during a couch shuttle pass m. In some variations, the radiation fluence may be derived based on the acquired PET imaging data that has been filtered by the ACF h, for example by convolving an image of the target anchor location derived from the cross-correlated PET imaging data with firing filters, or shifting the planned fluence map to the target anchor location derived from the cross-correlated PET imaging data. Further, the method 802 may include updating 864 the ACF h based on the acquired additional PET imaging data in step 862. In some variations, the acquired additional data may be the cumulation of PET imaging data acquired during the entire couch shuttle pass. Following the step 864, the method 801 includes delivering 866 radiation fluence during the next shuttle pass m+1 while acquiring additional PET imaging data. Similar to the step 862, the radiation fluence at step 866 may be derived based on the acquired PET imaging data that has been filtered (e.g., cross-correlated) by the updated ACF h. Further, the method 802 may include evaluating 868 whether the planned radiation dose for the treatment session has been delivered. If the planned radiation dose has been delivered (step 868, Yes) the method 802 proceeds to ending 870 the treatment session. Alternatively, if the planned radiation dose has not been delivered (step 868, No), the method 803 may comprise continuing radiation delivery (e.g., step 866), though some variations may optionally include updating the ACF h based on the additional acquired PET imaging data (e.g., the PET imaging data acquired at the step 862) before continuing radiation delivery.

In some cases, the method 802 optionally includes generating 872 a graphical representation of the PET imaging data (e.g., generating the graphical representation on a display associates with the BgRT imaging system) acquired during the BgRT treatment session. The step 872 may be similar to or the same as step 852 of method 801 or step 728 of method 700.

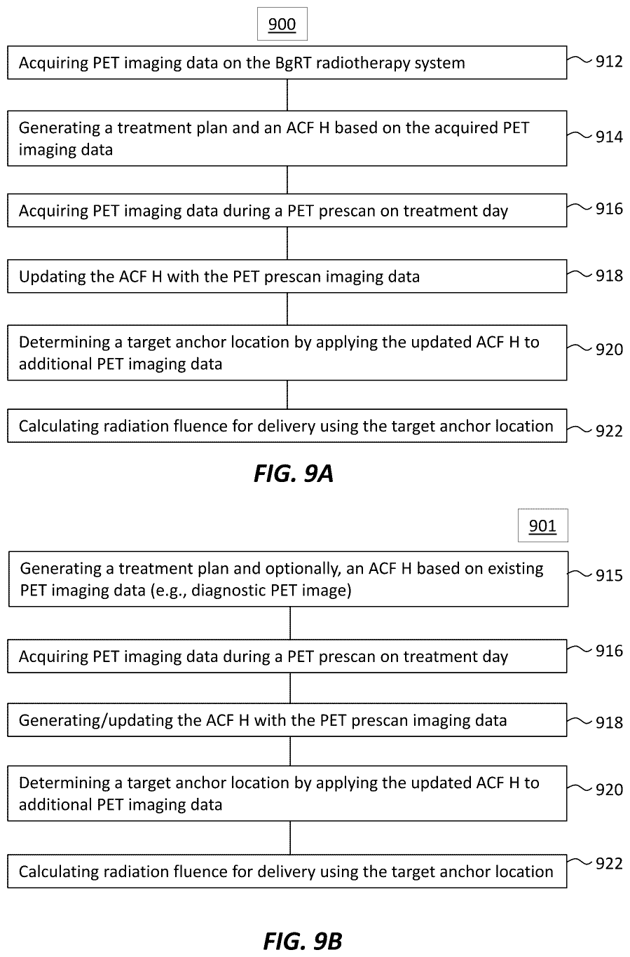

FIGS. 9A and 9B two variations of methods 900 and 901 for calculating radiation fluence for delivery using an adaptive correlation filter trained on different types of imaging data.

As described herein, after the ACF has been defined during treatment planning, it may be further updated (e.g., trained, adapted) using additional imaging data. In one variation, the ACF may be generated during treatment planning based on existing patient images that may not have been acquired on the PET (and/or CT) imaging system of a BgRT radiotherapy system, but then adapted on the day of treatment using imaging data acquired on the PET (and/or CT) imaging system of the BgRT radiotherapy system. Examples of existing patient images may include, but are not limited to, diagnostic imaging data obtained using a diagnostic PET imaging system, diagnostic CT imaging system, and/or a diagnostic MR imaging system. Alternatively, or additionally, in another variation, the ACF may be generated during treatment planning based on patient images that have been acquired on the BgRT radiotherapy system (e.g., during an additional PET imaging-only session). FIG. 9A depicts a flowchart representation of one variation of a method of treatment planning and delivery that includes the acquisition of imaging data on the BgRT radiotherapy system as part of treatment planning. Method 900 may include acquiring 912 PET imaging data on a BgRT radiotherapy system, and generating 914 a treatment plan and an adaptive correlation filter H based on the acquired PET imaging data. Before and/or on the day of the radiotherapy delivery session, method 900 may comprise acquiring 916 PET imaging data during a PET prescan, updating 916 the ACF H with the PET prescan imaging data (i.e., train the ACF H with the additional imaging data using any of the methods described herein), determining 920 a target anchor location by applying the updated ACF H to additionally acquired PET imaging data, and then calculating 922 the radiation fluence for delivery using the target anchor location.

In contrast, FIG. 9B depicts a flowchart representation of a method 901 that does not include an imaging session on the BgRT radiotherapy system as part of treatment planning. This method may help to streamline the overall workflow for the clinic by reducing the number of visits that a patient makes to the clinic and increasing BgRT radiotherapy system availability of radiotherapy delivery (instead of treatment planning). Method 901 may comprise generating 915 a treatment plan. Optionally, an adaptive correlation filter H may be generated based on existing PET imaging data, such as diagnostic PET/CT imaging data during treatment planning. Before and/or on the day of the radiotherapy delivery session, method 901 may comprise acquiring 916 PET imaging data during a PET prescan, and generating 918 the ACF H (or updating the ACF H if it was initially generated during planning) with the PET prescan imaging data (i.e., train the ACF H with the additional imaging data using any of the methods described herein), determining 920 a target anchor location by applying the updated ACF H to additionally acquired PET imaging data, and then calculating 922 the radiation fluence for delivery using the target anchor location. The ACF H may be generated using, for example, the methods described above in FIGS. 4A-4F, 5A-5C, and may be updated using, for example, the methods described above in FIG. 6.

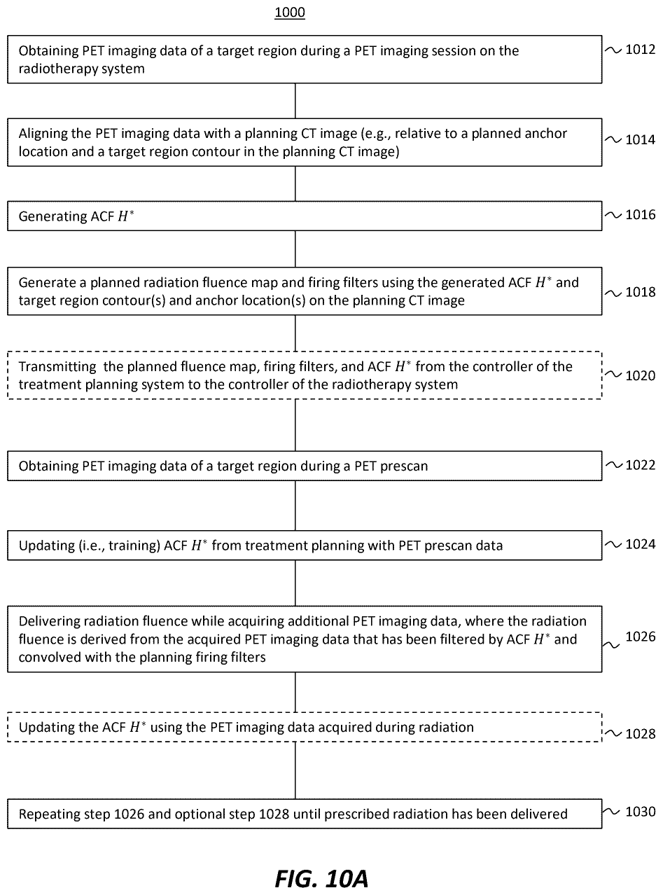

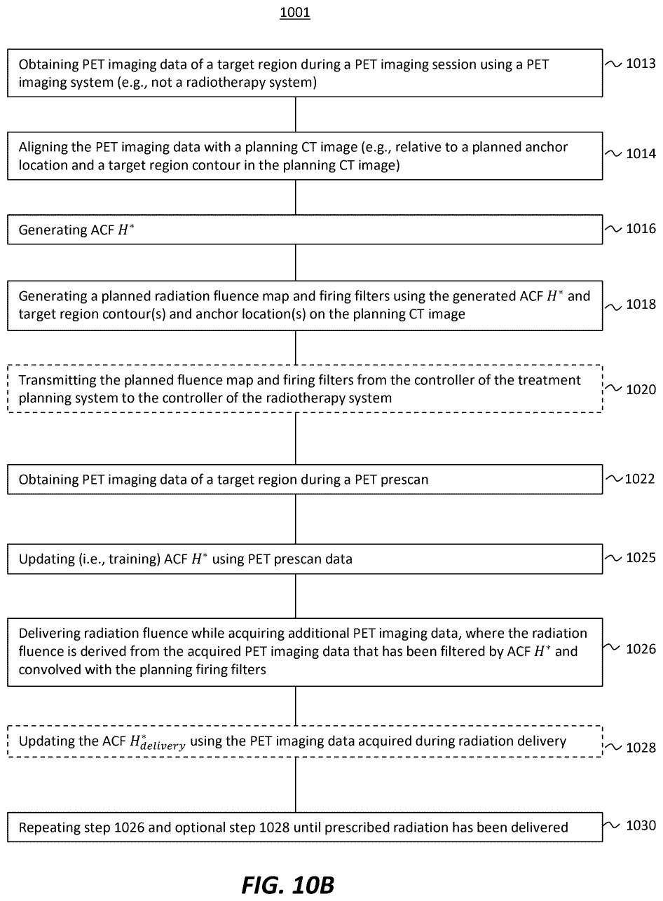

FIG. 10A depicts a flowchart representation of one variation of a method for BgRT treatment planning and delivery that includes a PET imaging session on the BgRT radiotherapy system. The ACF may be initially generated during BgRT treatment planning and then updated using PET prescan data (which is PET imaging data) and/or CT imaging data acquired at the start of a BgRT treatment session. Optionally, the ACF may be updated during the BgRT treatment session using the PET imaging data acquired during the treatment session. In one variation, method 1000 may comprise treatment planning steps 1012-1022 and radiation delivery (e.g., treatment session) steps 1024-1030 (noting that in some variations, obtaining PET imaging data during a prescan may be considered part of the radiation delivery session). The method 1000 may comprise obtaining 1012 PET imaging data of a target region during a PET imaging session using a BgRT radiotherapy system (e.g., the PET imaging system of a BgRT radiotherapy system may be used for obtaining the PET imaging data), and aligning 1014 the obtained PET imaging data with a planning CT image (e.g., relative to a planned anchor location and a target region contour in the planning CT image). The planning CT image may be a CT image obtained using a diagnostic CT imaging system (e.g., the CT imaging system not associated with the BgRT imaging system) or it may be obtained using a CT imaging system on the BgRT radiotherapy system. The planned anchor location relative to the defined target region contours may be selected by the medical professional, as described previously.

The method 1000 may further include generating 1016 ACF h using the obtained PET imaging data. The step 1016 may be, for example, the same as the method 400, as shown in FIG. 4A. Further, the method 1000 includes generating 1018 a planned radiation fluence map and firing filters. The method of generating a planned fluence map and firing filters described below may be used in any of the methods described herein. The planned fluence map is a radiation fluence map that when delivered, would provide the prescribed dose to the target region(s) and may be generated (e.g., calculated) through an iterative process using the dose prescription determined by a medical professional and a dose calculation matrix. In one variation, the dose prescription determined by the medical professional may include, for example, dose goals and objectives for a patient target region and/or OAR. Radiation dose (i.e., the amount of radiation absorbed by a subject) and radiation fluence FM (i.e., the amount of radiation emitted by a radiation source, and usually designated by radiation beams or beamlets) are related to each other (e.g., mapped to each other) by a dose calculation matrix A. That is,

$$D = A \cdot FM$$

A dose calculation matrix represents a dose contribution from each of a plurality of radiation beamlet to each voxel of a patient target region (and/or OAR). For example, a dose calculation matrix A may be a (k×n) matrix where n may be a number of possible radiation beamlets {bi} and k may be the number of pre-selected voxels for a patient target region. An i-th column of the dose calculation matrix A (which has k elements) represents a dose contribution from a unity-weighted beamlet bi to each of the k voxels. Dose calculation matrix A may be calculated column-by-column, for example, by ray-tracing each beamlet's aperture along the path through a patient target region and calculating the contribution of a unity-weighted beamlet to each of the k voxels. A beamlet aperture may be an MLC aperture defined by a single MLC leaf opening (i.e., of a binary MLC or a 2-D MLC). Examples of algorithms for calculating a dose calculation matrix that may be used in any of the methods described herein may include Monte-Carlo simulation, collapsed-cone convolution superposition, pencil-beam convolution, and others. Each patient target region and/or OAR may have its own dose calculation matrix.

The planned radiation fluence map FM may be the radiation fluence to be emitted to the patient at each firing position in order to deliver the prescribed dose D. In one variation of BgRT planning and delivery, the planned radiation fluence map FM may be represented by a firing filter p convolved with an image N of a function (e.g., delta function or Gaussian function, an image of a target anchor at its defined location) centered over the selected target anchor location:

$$D = A \cdot FM = A \cdot ( p * N )$$

In some variations, the image N may be imaging data (e.g., PET imaging data) of the target region. In addition to defining a dose prescription, a medical professional may set one or more constraints and/or cost or penalty functions C (D, FM) that specify characteristics of the dose distribution and/or radiation fluence map. Examples of cost functions may include, but are not limited to, minimum dose to target region, average or maximum dose on OARs, and/or fluence smoothness, total radiation output, total tissue dose, treatment time, etc. In one variation, a BgRT radiotherapy treatment planning system may be configured to calculate a radiation fluence map FM such that the dose prescription and constraints C (D, FM) are met. The radiotherapy treatment planning system may iterate through different radiation fluence values and/or radiation fluence maps to find the fluence values and/or fluence maps that minimize the cost function C (D, FM) while still meeting the dose prescription requirements. To calculate firing filters p, a radiotherapy treatment planning system may set up an optimization problem for minimizing the cost function C (D, FM), given a dose calculation matrix A and an image N of a function centered over the target anchor location. The anchor function in the image N used for this optimization may be the same anchor function that is used to generate an image of the target anchor location during a radiation delivery session. Calculating firing filters p may comprise iterating through different firing filter values such that the cost function C (D, FM) is minimized while still attaining dose goals and objectives in accordance with the dose prescription. Generating firing filters by iterating on an image of a target anchor (which may be a sharp peak at the anchor location) instead of imaging data (which may be noisy or diffuse) may help de-couple quality (e.g., conformality) of the radiation delivery from the quality of the imaging data acquired on the day of treatment. That is, the ACF may be applied to the newly acquired imaging data to identify the location of the anchor, which suppresses or removes imaging noise. The image of the anchor location, which has less noise than the acquired imaging data (and in some variations, no imaging noise at all), may then be convolved with the firing filters calculated here to generate the radiation fluence for delivery.

The method 1000 may optionally include transmitting 1020 the planned fluence map FM, the firing filters, and the ACF h from a controller of the system for planning a BgRT treatment to a controller (e.g., a suitable computing device, such as a processor, an integrated circuit, a microchip, and the like) of the BgRT radiotherapy system.

The method 1000 further includes obtaining 1022 new PET imaging data of a target region during a PET prescan. The PET prescan may be performed using a BgRT PET imaging system at the beginning of a BgRT treatment session. Additionally, the method 1000 includes updating 1024 the ACF h using the obtained new PET imaging data (e.g., the ACF h may be updated based on the newly acquired PET imaging data during the prescan). The updating 1024 of the ACF h may be performed using, for example, method 600. Additionally, or alternatively, the ACF may optionally be trained or updated using other PET imaging data, including, for example, simulated PET images, and/or simulated LORs for a corresponding PET image, either or both of which may include synthetic noise and/or PET signal variability based on models of tracer uptake characteristics, kinetics, and distribution, PET images from another imaging session or other imaging sessions (e.g., diagnostic images), synthetic PET images that simulate variability in PET tracer uptake characteristics, LOR count rate, shifts in the target region location, geometric changes to the target region (e.g., size, shape), background noise levels, or combination thereof.

The method 1000 further includes delivering 1026 radiation fluence while acquiring additional PET imaging data. In various cases, the radiation fluence is derived based on a filtered (or cross-correlation) image convolved with the planning firing filters, where the filtered image may be generated by applying the ACF to the acquired PET imaging data. As described throughout, the radiation fluence for delivery may be calculated based on an image of the target anchor location as determined from the filtered (or cross-correlation) image. In one variation, the location of the target anchor may be used to shift the planned fluence map. Alternatively, an image of the target anchor location (e.g., a Gaussian or delta function centered over the target anchor location) may be convolved with the firing filters calculated during treatment planning.

Further, the method 1000 may include an optional step of updating 1028 the ACF h using acquired additional PET imaging data. Similar to other updating steps, the updating 1028 of the ACF h may be performed using, for example, method 600.

In various cases, method 1000 may include repeating 1030 radiation delivery, i.e., step 1026, as well as optional step 1028 until a prescribed radiation dose is delivered to the target region during the BgRT treatment session.

FIG. 10B depicts a flowchart representation of one variation of a method for BgRT treatment planning and delivery that uses PET images that may be acquired as part of diagnosing and evaluating a patient's disease for BgRT treatment planning and ACF generation. In contrast to the method of FIG. 10A, the method of FIG. 10B does not require a separate PET imaging session on the BgRT radiotherapy system. The ACF generated during treatment planning is then further trained (e.g., updated) using the PET prescan imaging data acquired at the beginning of a BgRT treatment session. Additionally, or alternatively, the ACF may optionally be trained or updated using other PET imaging data, including, for example, simulated PET images, and/or simulated LORs for a corresponding PET image, either or both of which may include synthetic noise and/or PET signal variability based on models of tracer uptake characteristics, kinetics, and distribution, PET images from another imaging session or other imaging sessions (e.g., diagnostic images), synthetic PET images that simulate variability in PET tracer uptake characteristics, LOR count rate, shifts in the target region location, geometric changes to the target region (e.g., size, shape), background noise levels, or combination thereof. The method 1001 may include obtaining 1013 PET imaging data of a target region during a PET imaging session using a PET imaging system that may not be part of a radiotherapy system. The PET imaging system, for example, may be a diagnostic PET imaging system. In some variations, patients may already have diagnostic-quality CT images and/or diagnostic-quality PET images acquired as part of their disease evaluation and treatment planning. Steps 1014-1022 and 1026-1030 of method 1001 may be the same as the same numbered steps of method 1000.

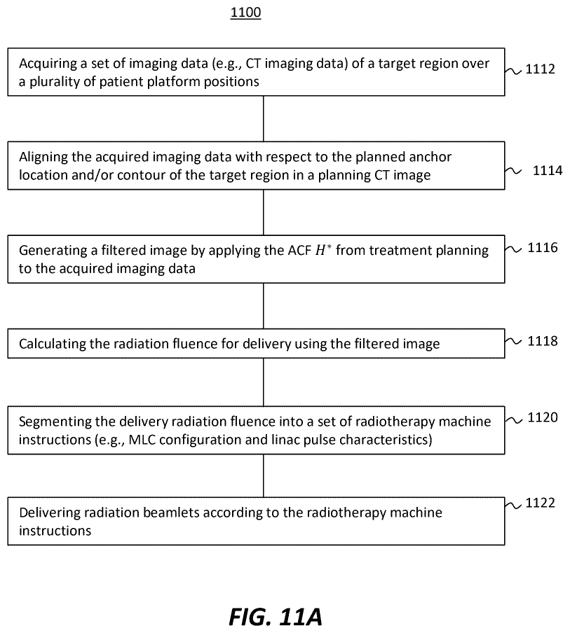

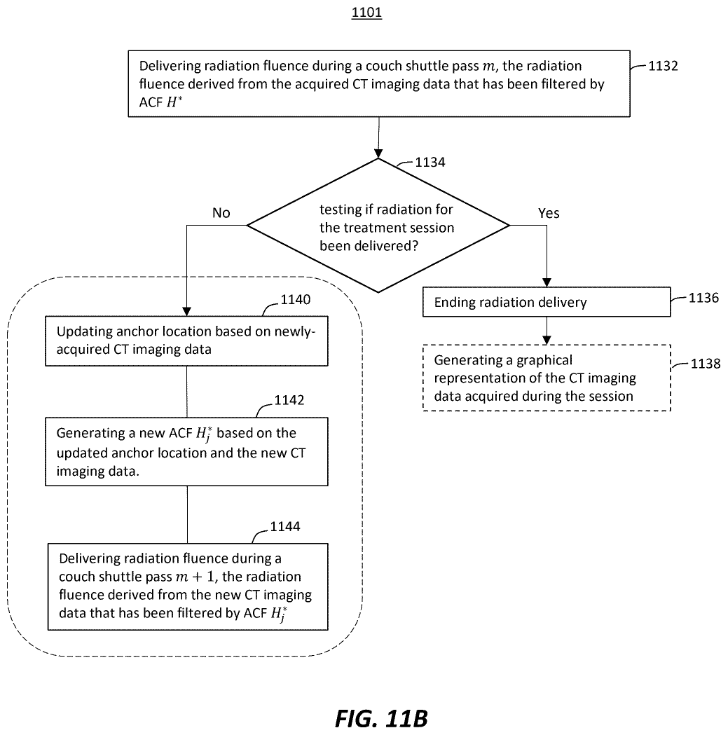

In some variations, the ACF may be used for image-guided radiotherapy treatments (e.g., for IMRT, SBRT, SRS treatments) when no PET images are acquired. For example, FIG. 11A shows an example method 1100 for delivering radiotherapy dose during a radiotherapy treatment session based on a CT image data. The method 1100 includes acquiring 1112 a set of imaging data (e.g., CT imaging data) of a target region over a plurality of patient platform positions and aligning 1114 the acquired CT imaging data with respect to a planned anchor location and/or contour of the target region in a planning CT image. The CT imaging data may be acquired at the beginning of the radiotherapy treatment session. In some variations, the acquired CT imaging data may comprise a CT image that is used for positioning the patient on the platform or couch (i.e., for localization, which may also be referred to as setup). The method 1100 includes generating 1116 a filtered image by applying an ACF h to the CT imaging data. The ACF h may be generated using a method 500 as described in relation to FIG. 5A. Further, the method 1100 includes calculating 1118 radiation fluence for delivery to a target region during the radiotherapy treatment session by using the filtered image. For example, the step 1118 may be performed by convolving the filtered image with a firing filter. In some variations, calculating the radiation fluence for delivery may comprise convolving an image of the target anchor location (generated from the filtered image using any of the methods described herein) with a firing filter. Alternatively, the step 1118 may be performed by shifting a planned radiation fluence to a target anchor location. The target anchor location may be determined using the ACF h and the CT imaging data (e.g., the target anchor location may be determined as a coordinates of a pixel having a maximum value in the filtered image).

Method 1100 may comprise segmenting 1120 the delivery radiation fluence into a set of radiotherapy machine instructions (e.g., MLC configuration and linac pulse characteristics), and delivering 1128 radiation beamlets according to the radiotherapy machine instructions.

FIG. 11B shows an example method 1101 which may be a variation of method 1100 and applied when multiple couch shuttles passes are used for delivering radiation dose during a radiotherapy treatment session. Method 1101 may optionally comprise calculating a new ACF based on updates to the target anchor location and target region contour(s) that have been changed according to the newly acquired CT imaging data. Since the relative position between the target anchor and the target region contour(s) may be changed, a new ACF may be generated (instead of updating the existing ACF which was generated based on a previous relative position between the target anchor and the target contour). The method 1101 may include delivering 1132 radiation fluence during a couch shuttle pass m. In some variations, the radiation fluence may be determined using the acquired CT imaging data that has been filtered by an ACF H. The ACF H may be generated using, for example, method 500.

Further, the method 1101 includes testing 1134 whether the planned radiation dose for the treatment session has been delivered. If the planned radiation dose has been delivered (step 1134, Yes), the method 1101 includes ending 1136 the treatment session. Alternatively, if the radiation dose has not been delivered (step 1134, No), the method 1101 may optionally include acquiring new CT images and updating 1140 a target anchor location based on the new acquired CT images. In an example implementation, the target anchor location may be updated by first generating a filtered image. The filtered image may be generated by cross-correlating the ACF H with a CT image. The target anchor location may be then obtained as a location of a pixel of the filtered image that exhibits a maximum value.

The method 1101 also includes generating 1142 a new ACF

$${H}_{j}^{*}$$

based on the updated target anchor location and newly acquired CT images, as described, for example, by method 500 and/or method 400.

The method 1101 further includes delivering 1144 the radiation fluence during the next shuttle pass, i.e., a couch shuttle pass m+1. The radiation fluence may be determined based on the new CT imaging data and the new ACF

$${H}_{j}^{*} .$$

For example, the radiation fluence may be determined by generating a filtered image and convolving the filtered image with a firing filter. The filtered image may be generated by cross-correlating the ACF

$${H}_{j}^{*}$$

with a CT image obtained using the CT imaging data.

While the examples and variations of methods described above comprise using a single target anchor per target region for treatment planning and delivery, other examples and variations may comprise using a plurality of target anchors (i.e., two or more) per target region for treatment planning and delivery. For example, some methods may comprise defining three target anchors for a target region contour, where the three target anchors together define a plane, such as a target orientation plane (which may be referred to as the planning orientation plane). The target orientation plane may have a particular orientation in 3-D space relative to a fixed coordinate system, for example, the patient coordinate system and/or the radiotherapy system coordinate system. A correlation filter, such as an ACF described herein, may be calculated for each of the target anchors based on their relative position to the target region contour. During the radiation delivery session, imaging data may be acquired and the ACF for each target anchor may be applied to (e.g., cross-correlated with) the acquired imaging data, and the cross-correlated image may be used to determine the location of each target anchor. Once the locations of the three target anchors have been determined (e.g., using 3-D coordinates, for example, (x, y, z) coordinates), the three target anchors may define the target orientation plane during radiation delivery (which may be referred to as the delivery orientation plane). The delivery orientation plane and the planning orientation plane may be compared to determine whether the target region shifted and/or rotated and/or tilted (i.e., any 3-D orientation changes) on the day of delivery relative to its position during planning. The shifts and/or rotations and/or tilts may then be applied to the planned fluence map to calculate the delivery fluence map. In some variations, the delivery fluence map (i.e., which is the planned fluence map that has adapted according to the changes in the delivery orientation plane) may be segmented into radiotherapy machine instructions during the delivery session, optionally in real-time, and then executed by the radiotherapy machine to deliver the prescribed radiation to the target region. Adjusting the radiation fluence according to the rotation and/or tilt, in addition to the shift, of a target region during a treatment session may help improve the accuracy and conformality of the dose distribution so that the prescribed dose is applied to the target.

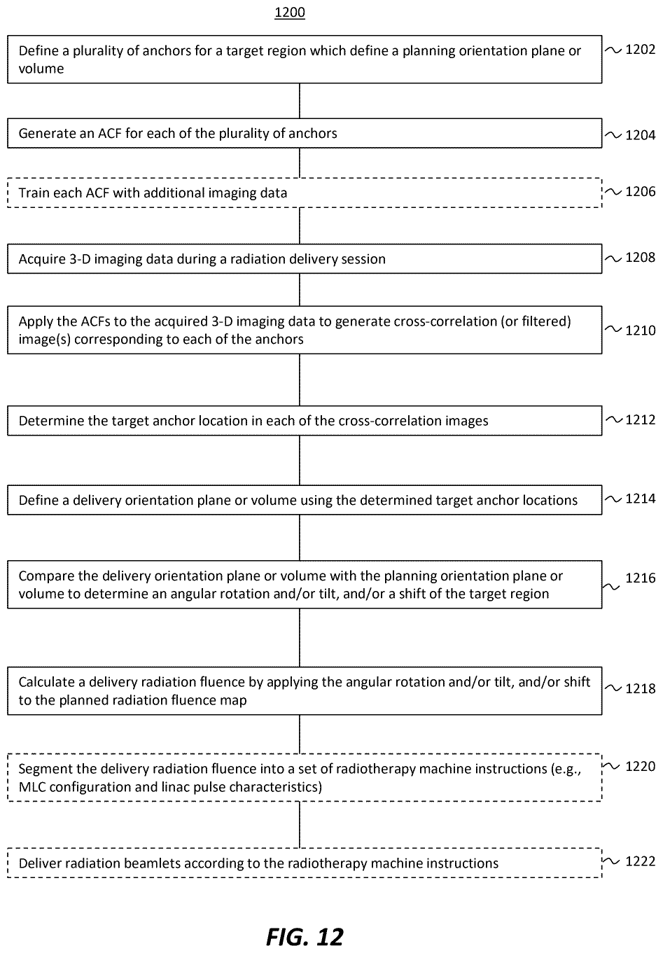

FIG. 12 is a flowchart depiction of one variation of a method of defining multiple anchors for a target region, calculating multiple corresponding correlation filters (e.g., ACFs) for each anchor, and then using the correlation filters during radiation delivery to determine orientation changes in the target so that the delivered fluence accounts for those orientation changes. Method 1200 may comprise treatment planning steps (e.g., steps 1202-1206) and radiation delivery steps (e.g., steps 1206-1222), where the optional training of the ACFs on additional imaging data may take place before and/or during the radiation delivery session. In one variation, method 1200 may comprise defining 1202 a plurality of anchors for a target region, generating 1204 an ACF for each of the plurality of anchors, and optionally, training 1206 each ACF with additional imaging data. The target anchors may be selected such that they define a planning orientation plane and/or volume, and their location may be defined relative to the target region contour, as explained above. The anchors and the target region contour may be defined on one or more sets of imaging data, including for example, planning CT images, planning MR images, planning PET images, etc. In some variations, the target anchors may be selected to be the centroid of a target region (e.g., as explained in the examples above), any distinctive feature that can be determined from imaging data, and/or any arbitrary location relative to the target region contour. During treatment planning, the orientation of the planned fluence map may be defined relative to the defined target anchors such that knowing the orientation of the target anchors provides information on how to adjust the planned fluence map to account for any orientation changes.

After an ACF for each target anchor has been generated and optionally trained on additional imaging data, method 1200 may comprise acquiring 1208 imaging data (e.g., 3-D imaging data), applying 1210 the ACFs to the acquired 3-D imaging data to generate cross-correlation (or filtered) image(s) corresponding to each of the anchors, determining 1212 each of the plurality of target anchor locations in the cross-correlation images, and defining 1214 a delivery orientation plane or volume using the determined target anchor locations. The generation of cross-correlation (or filtered) images for each of the target anchors and methods of identifying the target anchor location in the cross-correlation images may be similar to any of the methods described above, e.g., FIGS. 3A-B, 7B-C, etc. For example, determining 1212 the target anchor locations may comprise locating regions in the cross-correlation images that have pixel or voxel intensities that are higher than a predetermined threshold. Alternatively, or additionally, determining 1212 a target anchor location may comprise defining a contour of the target region based on the cross-correlation image and identifying the distinctive features or landmarks on the target region contour that correspond with the defined target anchors. In some variations, the distinctive features or landmarks may be an anatomical structure (e.g., bone) or it may be geometrically derived from the contour of the target region. For example, during planning, a first target anchor may be defined as the midpoint of a line segment that spans the widest portion of the target region contour, and a second target anchor may be defined as the midpoint of a line segment that spans the narrowest portion of the target region contour. Optionally, a third target anchor may be the centroid of the target region contour. When the cross-correlation image is generated during radiation delivery, the method may comprise identifying the first target anchor by drawing a line segment that spans the widest portion of the target region contour and finding its midpoint, identifying the second target anchor by drawing a line segment that spans the narrowest portion of the target region contour and finding its midpoint, and optionally, calculating a centroid of the target region contour. In situations where the acquired imaging data and/or cross-correlation image is not of sufficient quality to determine the location of all of the target anchor locations, the method may comprise generating a notification to the operator that includes which target anchors can be located and which ones cannot, so that the operator can decide whether to pause the radiation delivery session. In one variation, the operator may be presented with the option to select the target anchor whose location is known with the highest level of certainty as the target anchor upon which to generate the delivery radiation fluence. For example, if the location of the target anchor at the centroid of the target contour is known with the greatest certainty, and/or if the location of the off-centroid anchors are not know with sufficient certainty, the delivery radiation fluence may simply be shifted according to the location of the centroid target anchor (e.g., the high intensity pixels in the center of the cross-correlation image). The delivery radiation fluence would not be adjusted for any rotation and/or tilt of the target region.

After the delivery orientation plane or volume is defined, method 1200 may comprise comparing 1216 the delivery orientation plane or volume with the planning orientation plane or volume to determine an angular rotation and/or tilt, and/or a shift, and calculating 1218 a delivery radiation fluence by applying the corresponding angular rotation and/or tilt, and/or shift to the planned radiation fluence map. Optionally, method 1200 may comprise segmenting 1220 the delivery radiation fluence into a set of radiotherapy machine instructions (e.g., MLC configuration and linac pulse characteristics) and delivering 1222 radiation beamlets according to the radiotherapy machine instructions.

In a first example, a method may comprise selecting three anchors for a target region contour. The first anchor may be defined at a centroid of the target region contour, the second anchor may be defined as the midpoint of a line segment that spans the widest portion of the target region contour, and a third target anchor may be defined as the midpoint of a line segment that spans the narrowest portion of the target region contour. Alternatively, in a second example, each of the three anchors may be defined on orthogonal planes in a 3-D coordinate system, and/or projections of the target region contour on orthogonal planes in a 3-D coordinate system. For example, in a 3-D coordinate system with X, Y, and Z axes, the first anchor may be defined at a point or landmark on the X-Y plane, the second anchor may be defined at a point or landmark on the X-Z plane, and the third anchor may be defined at a point or landmark on the Y-Z plane. Landmarks may be features in the image that have a unique pattern (e.g., distinctive anatomical features or shapes), and/or high-intensity pixels or voxels, and the like. Three filters or templates (e.g., ACFs) may be generated for the three anchors. During a radiation delivery session, an image of the target region may be cross-correlated with (i.e., filtered by) the three filters or templates to generate three cross-correlation (or filtered) images. The locations of the three anchors may be identified as described above. For the first example, the method may comprise calculating the centroid of a first cross-correlation image to identify the location of the first anchor, calculating the midpoint of the line segment spanning the widest dimension of the target contour to identify the location of the second anchor, and calculating the midpoint of the line segment spanning the narrowest dimension of the target contour to identify the location of the third anchor. For the second example, the method may comprise projecting the imaging data to each of the 3-D orthogonal planes (i.e., X-Y plane, X-Z plane, Y-Z plane) and determining the location of the point or landmark on each plane to identify the location of the first anchor, second anchor, and third anchor. Once the target anchor locations are identified, the method may comprise defining 1214 a delivery orientation plane or volume using the three target anchor locations, comparing 1216 the delivery orientation plane or volume with the planning orientation plane or volume, and then calculating 1218 a delivery radiation fluence that is adapted to include the changes in the orientation plane or volume.

Another variation of a method for determining orientation changes in the target may not require the use of multiple anchors for a target region. To determine the rotation and/or tilt of a target region, the method may comprise iteratively (e.g., repeatedly) rotating and/or tilting an ACF template that has been calculated for a target region, applying each of the rotated and/or tilted ACF templates to the acquired imaging data of the target region to generate a plurality of filtered images, and evaluating the response (i.e., intensity, magnitude) of each of the filtered images. The ACF template may be calculated (using the methods described herein) for the original or initial position and orientation of the target region, for example, the position and orientation at treatment planning and/or before the treatment session. Evaluating the response of the filtered images may comprise comparing all of the filtered images and identifying the filtered image with the largest response, and the rotation and/or tilt of the ACF template that gave rise to the filtered image with the largest response may be the rotation and/or tilt of the target region from its original position. This rotation and/or tilt may then be applied to planned fluence map so that the delivered fluence accounts for those orientation changes. Since the ACF template functions to identify portions of the imaging data that have the highest correlation with the training data set (i.e., the original target region position), testing different ACF template rotations on the imaging data and finding which ACF template rotation has the largest response may provide an estimate of the rotation of the target region. This method may similarly be used to determine a shift of the target region. Instead of iteratively (e.g., repeatedly) rotating an ACF template, the method may comprise iteratively shifting the position of ACF template, applying each of the shifted ACF templates to the acquired imaging data of the target region to generate a plurality of filtered images, and evaluating the response (i.e., intensity, magnitude) of each of the filtered images. Evaluating the response of the filtered images may comprise comparing all of the filtered images and identifying the filtered image with the largest response, and the shift the ACF template that gave rise to the filtered image with the largest response may be the shift of the target region from its original position.

### Workflow Methods for Biology-Guided Radiotherapy (BgRT)

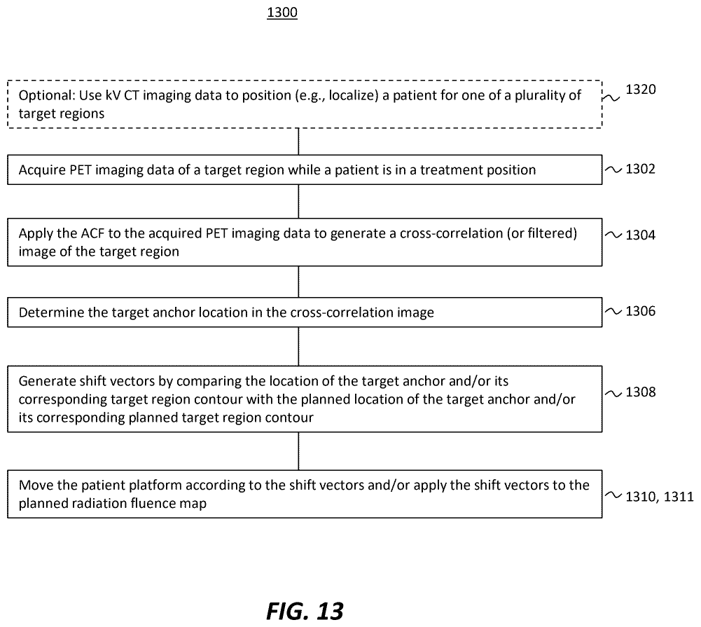

The BgRT treatment planning methods described herein comprising the generation of an ACF based on a target (or OAR) region contour and corresponding target (or OAR) region contour and generating firing filters based on an image of the target (or OAR) region anchor at a planned location may be used to facilitate the localization of a patient at the start of a treatment session. Patient localization is a process by which a patient is positioned in the same position they were in when the planning image was acquired. In some variations, a planning image may be a CT image and so for localization, the patient is positioned on the treatment system based on CT imaging data acquired on the day of treatment and aligned to the planning CT image. Alternatively, or additionally, the methods described herein may be used for patient localization using PET imaging data. That is, the PET imaging data acquired on the day of treatment, e.g., at the beginning of a treatment session, may be processed using the methods described herein using an adaptive correlation filter that results in an image that facilitates the identification of the location of the target anchor. The location of the target anchor determined on the day of treatment may be compared with the location of the target anchor during planning. Any offset(s) between the target anchor locations may then be applied to the patient (e.g., by moving the patient platform or adjusting the patient's position on the platform) and/or the planned radiation fluence. One variation of a method for patient localization using PET imaging data and an adaptive correlation filter is depicted in FIG. 13. Method 1300 may comprise acquiring 1302 PET imaging data of a target region while a patient is in a treatment position, applying 1304 the ACF to the acquired PET imaging data to generate a cross-correlation (or filtered) image of the target region, determining 1306 the target anchor location in the cross-correlation image, generating 1308 shift vectors by comparing the location of the target anchor and/or its corresponding target region contour with the planned location of the target anchor and/or its corresponding planned target region contour, and moving 1310 the patient platform according to the shift vectors. Alternatively, or additionally, method 1300 may comprise applying 1311 the shift vectors to the planned radiation fluence map. Since shifting the planned radiation fluence map 1311 does not involve physically moving the patient platform, this may be referred to as “virtual localization”. Optionally, localization may include a combination of kV CT imaging-based localization and PET imaging-based localization. That is, method 1300 may optionally comprise using 1320 kV CT imaging data to position (e.g., localize) a patient for one or more of a plurality of target regions, and the one or more remaining target regions may be localized using the PET imaging-based method 1302-1311. In some variations, the method 1300 may be performed serially for multiple target regions. For example, steps 1302-1311 (and optionally 1320) may be performed for a first target region, radiotherapy provided to the first target region, and then repeated for a second target region (with a second ACF applied to the imaging data to determine the second target anchor location), followed by radiotherapy provided to the second target region. In certain variations, the second ACF may be applied to the initially acquired PET imaging data or it may be applied to newly acquired PET imaging data, such as PET imaging data acquired during the irradiation of the first target region. In another variation, a first target region may be physically localized (e.g., steps 1302 or 1320, 1304, 1306, 1308, and 1310) while a second target region may be virtually localized (e.g., steps 1302, 1304, 1306, 1308, and 1311). For example, one or more of a plurality of target regions may be physically localized (involving motion of the patient platform and/or patient position on the platform), and the one or more remaining target regions may be virtually localized (solely shifting the planned fluence map).

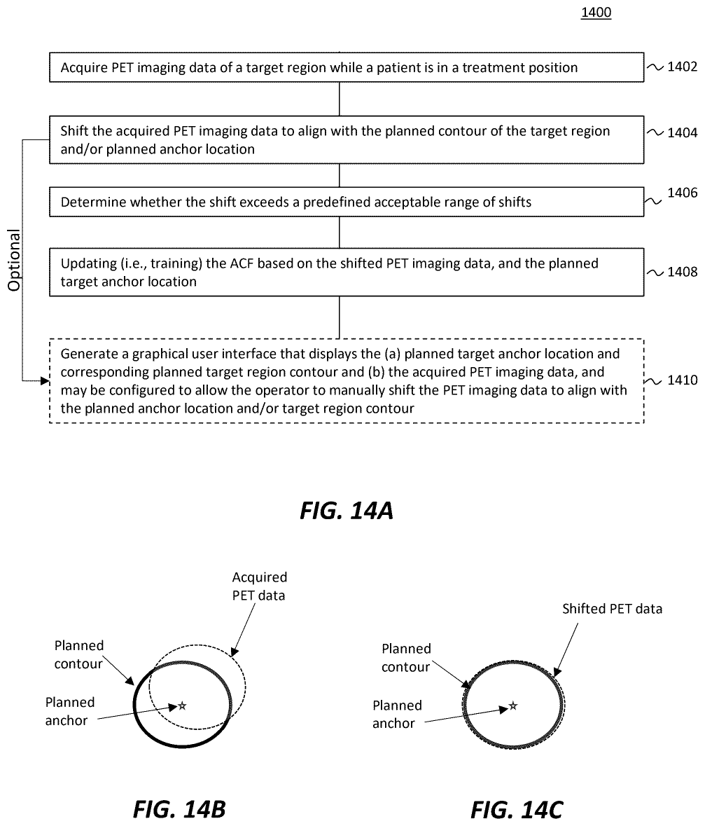

As described herein, the ACF may be updated periodically before or during radiation delivery to improve its accuracy in identifying the target tumor(s) (and/or OARs). Since BgRT utilizes PET signals from an injected PET tracer that accumulates at tumors to help guide the radiation delivery, the ACF may be generated and/or updated using PET imaging data acquired on the day of radiation delivery. This allows the ACF to be trained to find a tumor's position even if the characteristics of the PET imaging data (e.g., SUV, intensity distribution, tracer uptake profile or distribution, noise levels, etc.) vary from previously-acquired PET imaging data. Some variations of updating the ACF to account for day-of PET imaging data variability may include safety checks that alert an operator if the PET imaging data has deviated or varied too much from what is expected or acceptable. Optionally, some variations may include generating a graphical user interface to allow operator involvement in updating or generating an ACF. FIG. 14A depicts one variation of a method of updating an ACF that includes a check as to whether the PET imaging data has changed more than an acceptable range, e.g., the PET data has shifted beyond an acceptable threshold from its location at an earlier PET imaging session (e.g., during planning). Method 1400 may comprise acquiring 1402 PET imaging data of a target region while a patient is in a treatment position, shifting 1404 the acquired PET imaging data to align with a planned contour of the target region and/or a planned anchor location, determining 1406 whether the shift exceeds a predefined acceptable range of shifts, and updating 1408 (or in some variations, generating) an adaptive correlation filter (ACF) based on the shifted PET imaging data and the planned target anchor location. In some variations, if it is determined 1406 that the offset that is needed to shift the PET data into alignment with the planned target region contour exceeds an acceptable range of shifts, method 1400 may comprise generating a notification to the operator. The notification may be a graphical notification and/or an audible notification, which may indicate that the shift or offset amount is outside of the acceptable range and that the operator may wish to intervene. Possible operator interventions may include, but are not limited to, pausing (i.e., not proceeding with) the radiation delivery session, re-scanning the patient (i.e., acquiring a new set of PET imaging data), and/or adjusting the patient's position (e.g., by moving the patient platform and/or adjusting the patient's position on the patient platform).

Optionally, method 1400 may comprise generating 1410 a graphical user interface that displays the (a) planned target anchor location and corresponding planned target region contour and (b) the acquired PET imaging data. In some variations, the graphical user interface may be configured to allow the operator to manually shift the PET imaging data to align with the planned anchor location and/or target region contour. FIG. 14B depicts one example of a graphical user interface that depicts the acquired PET imaging data overlaid on a planned contour and anchor at the planned location. If the acquired PET imaging data is shifted from the planned contour and/or planned anchor, the graphical user interface may comprise control indicia that allows the operator to manually shift the PET imaging data to align with the target region contour. Examples of control indicia may include a graphical element having a first mode that allows the user to select the PET imaging data and a second mode that allows the user to drag the PET imaging data to the desired location (e.g., aligned with the target region contour). The graphical element may be a geometric shape (e.g., circle, triangle, square, etc.) or an icon (e.g., arrow, hand, cursor indicator, pointer, etc.). Optionally, the graphical user interface may include guidance indicators that suggest to the operator a direction in which they may wish to shift the PET imaging data. The guidance indicators may be, for example, arrows indicating the direction, and may optionally flash or flicker to be distinctive over the background graphics. FIG. 14C depicts the graphical user interface of FIG. 14B after the PET imaging data has been shifted to align with the target region contour. If the PET imaging data is already aligned with the planned target region contour, then no shifting of the PET imaging data, manual or automatic, needs to be done. The ACF may be updated 1408 using the shifted PET imaging data and the planned anchor location (e.g., as depicted in FIG. 14C), as described above (e.g., method 600 of FIG. 6).

While the examples above describe using an ACF to identify target regions and anchor locations for a therapeutic session, for example a BgRT radiotherapy session and/or an image-guided radiotherapy session (e.g., IMRT, SBRT, SRS), any of the methods described herein may be used in a non-therapeutic radiation delivery session. For example, the methods described herein may be used for a quality assurance (QA) radiation delivery session where radiation is delivered to a phantom and/or radiation measurement devices, and measurements of the emitted radiation are used to evaluate the quality of the radiotherapy treatment plan as well as the function of the radiotherapy system (i.e., mechanical and electronic functions are performing within specification to deliver the radiation designated by the radiotherapy treatment plan). BgRT treatment plans may be evaluated for quality by translating the corresponding planned fluence map into machine instructions and then executing those machine instructions on a BgRT radiotherapy system with a phantom on the patient platform. The phantom may comprise measurement devices, such as radiation sensors and/or film, that measure the radiation emitted. The radiation measurements may then be used to calculate the delivered dose distribution, which is compared to an expected (e.g., the prescribed) dose distribution. However, this method of BgRT treatment plan QA does not include the acquisition of PET imaging data, convolving the PET imaging data with BgRT treatment plan firing filters to derive the radiation fluence for delivery, and then segmenting the derived fluence for delivery to the phantom. This method of BgRT radiation delivery may be referred to as “convolutional firing” because the radiation fluence for delivered is derived by convolving real-time acquired imaging data (e.g., PET imaging data) with firing filters. The firing filters may be shift-invariant, which results in a delivered radiation fluence that follows the tumor target as it moves. In order to QA the convolutional firing aspect of BgRT delivery, the QA session needs to provide PET imaging data that may be used by the BgRT system to derive the delivery fluence. Since it can be difficult to re-create a PET-emitting source that emits positrons in a similar manner to a PET tracer within a patient, QA of the convolutional firing aspect of a BgRT radiotherapy plan may be challenging. However, with the methods described herein where the firing filters are derived from an image of a target anchor location, and not from PET imaging data, the convolutional firing aspects of a BgRT delivery may be tested using a positron-emitting point source. The “focal” emission of positrons from a point source may be a reasonable approximation of an image of a target anchor location derived from PET images filtered by an ACF. This may facilitate the QA evaluation of a patient-specific BgRT treatment plan using a positron-emitting point source.

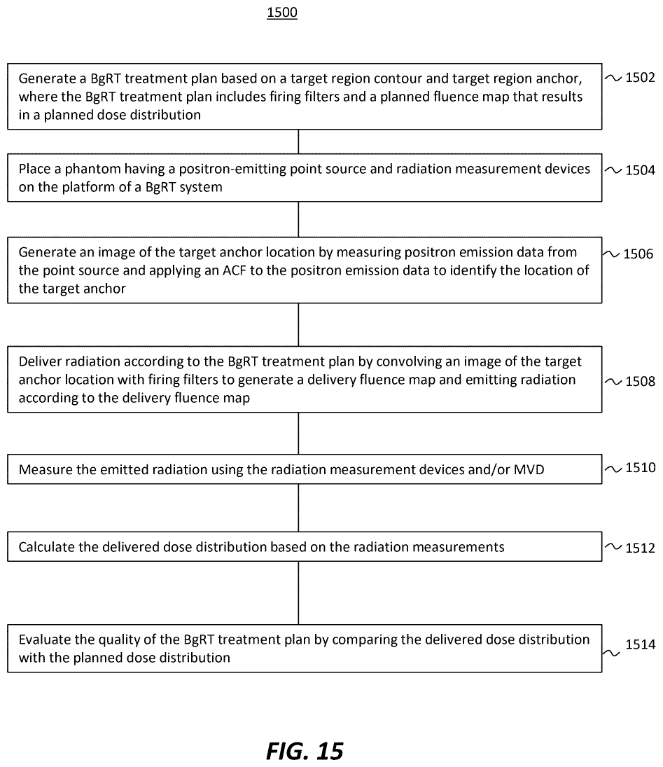

One example of a quality assurance (QA) method for a BgRT treatment plan is depicted in the flowchart of FIG. 15. Method 1500 may comprise generating 1502 a BgRT treatment plan based on a target region contour and target region anchor, where the BgRT treatment plan includes firing filters and a planned fluence map that results in a planned dose distribution, placing 1504 a phantom having a positron-emitting point source and radiation measurement devices on the platform of a BgRT system, generating 1506 an image of the target anchor location by measuring positron emission data from the point source and applying an ACF to the positron emission data to identify the location of the target anchor, delivering 1508 radiation according to the BgRT treatment plan by convolving an image of the target anchor location with firing filters to generate a delivery fluence map and emitting radiation according to the delivery fluence map, measuring 1510 the emitted radiation using the radiation measurement devices and/or MV detector, calculating 1512 the delivered dose distribution based on the radiation measurements, and evaluating 1514 the quality of the BgRT treatment plan by comparing the delivered dose distribution with the planned dose distribution. The ACF may be generated using any of the methods described above (e.g., referring to FIGS. 4A-4F and FIGS. 5A-D). Optionally, in some variations, the method 1500 may comprise calculating the planned radiation dose to the phantom using a CT image of the phantom, and evaluating 1514 the quality of the BgRT plan by comparing the measured radiation dose with the planned radiation dose to the phantom. This method of BgRT treatment plan QA may comprehensively test the quality of most, if not all, aspects of BgRT delivery, including the quality of the BgRT planned fluence map, PET detector functionality, computational robustness and speed (for the convolving the firing filter(s) with the images of the target anchor location) of calculating the fluence map for delivery, segmentation into machine instructions, and finally, emitting radiation according to the machine instructions (which includes proper functioning of the linac, beam-shaping components, gantry motion, and the like). The above is an example of a non-therapeutic use of the methods described herein.

The examples provided herein use an adaptive correlation filter as a tracking filter to facilitate the determination of an estimated location of a target region, however, it should be understood that in some variations, any tracking filter or template may be used to determine an estimated location of the target region. For example, the tracking filter or template may be defined during treatment planning and/or at the beginning of a treatment session, and not updated or trained on additional imaging data. The tracking filter, without any further training or updates, may be applied to imaging data acquired during a radiation delivery session.

Aspects of the present disclosure also describe a radiation therapy system similar or the same as the system 100, as shown in FIGS. 1A and 2A-2B. The radiation therapy system may include a rotatable gantry, a therapeutic radiation source mounted on the gantry, and one or more imaging sensors mounted on the gantry to acquire imaging data of a target region. The imaging sensors may be configured to acquire PET imaging data and, in some cases, CT imaging data. Further, the radiation therapy system may include a controller in communication with the gantry, the therapeutic radiation source, and the one or more imaging sensors. The controller may be any suitable computing device such as a microprocessor, integrated circuit, computer cluster, and the like. The controller may include a memory for storing instructions to be executed by the computing device of the controller. The controller may be configured to generate a target anchor location by applying an adaptive correlation filter to acquired imaging data. The target anchor location may be obtained by cross-correlating the adaptive correlation filter with at least one image obtained using acquired imaging data. Further, the controller is configured to calculate a radiation fluence to be delivered to the target region by convolving the target anchor location with a firing filter, as described above, for example, by step 752 of method 701 or by step 1118 of method 1100.

Further, aspects of the present disclosure also describe a system for generating a tracking filter, such as an adaptive correlation filter, using suitable imaging data (e.g., CT imaging data and/or PET imaging data). The system may be a computing system that may include a memory for storing imaging data and instructions and a processor configured to execute the instructions to perform various operations. The operations may include receiving an input from a user to select a planned target anchor location based on a contour of a target region in CT imaging data. The planned target anchor location may be determined based on any suitable approaches described herein (e.g., the planned target anchor location may be determined as a centroid of the contour of the target region obtained using CT image data as described in step 413 of method 400). Further, the operations may include aligning the PET imaging data with respect to the contour of the target region (e.g., such aligning may be performed using step 415 of method 400) and defining an image of an anchor function centered on the planned target anchor location. The image of the anchor function may be determined numerically as an image representation of a numerical delta function or a Gaussian function, as described above in relation to method 701. Alternatively, or additionally, the anchor function may comprise a plurality of delta functions (or Gaussian functions) where the peaks are in proximity to the target anchor location. Further, the operations may include generating an ACF h using the PET imaging data and the image of the anchor function. The step of generating the ACF h may be the same as method 400 or method 500, as described above.

In some variations, the system may include a display for displaying a graphical user interface for interacting with the processor. In some cases, the processor is configured to execute instructions to generate signals for displaying a graphical user interface (GUI) that includes CT imaging data and/or PET imaging data and to output the signals to the display for displaying GUI on the display. In some cases, the GUI includes a graphical representation of the planned target anchor location and/or image of the anchor function depicted simultaneously with the contour of the target region.

While various variations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described systems and methods can be implemented in any of numerous ways. For example, at least some methods of the present technology may be implemented using hardware, firmware, software, or a combination thereof. For example, various controllers or computing devices described herein may be implemented using hardware, firmware, software, or a combination thereof. When implemented in firmware and/or software, the firmware and/or software code can be executed on any suitable processor or collection of logic components, whether provided in a single device or distributed among multiple devices.

In this respect, various aspects described herein may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various variations of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of variations as discussed above. Additionally, it should be appreciated that according to one aspect, one or more computer programs that when executed perform methods of the present invention need not reside on a single computer or processor but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various variations.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of radiation pointers, tags or other mechanisms that establish relationship between data elements.

Also, various inventive concepts may be embodied as one or more methods, of which an example has been provided. The acts performed as part of the method may be ordered in any suitable way. Accordingly, variations may be constructed in which acts are performed in an order different than illustrated, which may include performing some acts simultaneously, even though shown as sequential acts in illustrative variations.

1. A method for determining a radiation fluence for delivery, the method comprising:
acquiring imaging data of a target region;
generating a filtered image by applying an adaptive correlation filter to the imaging data;
determining a target anchor location from the filtered image; and
calculating a radiation fluence to be delivered to the target region using the target anchor location.

2. The method of claim 1, wherein calculating the radiation fluence comprises applying a firing filter to an image of the target anchor location, wherein the firing filter is calculated based on previously-acquired imaging data of the target region.

3. The method of claim 1, wherein calculating the radiation fluence comprises shifting a planned fluence according to the target anchor location.

4. The method of claim 1, further comprising delivering the calculated radiation fluence to the target region with a therapeutic radiation source.

5. The method of claim 1, further comprising updating the adaptive correlation filter with additional imaging data.

6. The method of claim 5, further comprising acquiring the additional imaging data and updating the adaptive correlation filter before generating the filtered image.

7. The method of claim 6, wherein the additional imaging data is simulation imaging data.

8. The method of claim 1, wherein the acquired imaging data is obtained using a positron emission tomography (PET) imaging system.

9. The method of claim 1, further comprising:
determining a location of a planning contour using the acquired imaging data and wherein determining the target anchor location comprises locating the target anchor relative to the location of the planning contour.

10. The method of claim 9, wherein the planning contour is the boundary of a planning target volume (PTV), and the target anchor location is a location of a centroid of the PTV.

11. The method of claim 9, wherein the planning contour is the boundary of a planning target volume (PTV), and the target anchor location is a point on or within the boundary of the PTV.

12. The method of claim 9, wherein the planning contour is the boundary of an organ-at-risk (OAR), and the target anchor location is a point on or within the boundary of the OAR.

13. The method of claim 1, further comprising:
delivering the calculated radiation fluence;
acquiring additional imaging data;
updating the target anchor location;
calculating a second radiation fluence using the updated target anchor location; and
delivering the second radiation fluence.

14. The method of claim 13, wherein:
the acquired imaging data is obtained using a diagnostic positron emission tomography (PET) imaging system; and
the acquired additional image data is obtained using a biology-guided radiotherapy (BgRT) PET imaging system.

15. The method of claim 3, wherein the planned fluence comprises a segmented multi-leaf collimator (MLC) leaf pattern and calculating the radiation fluence comprises shifting the segmented MLC leaf pattern according to the target anchor position.

16. The method of claim 1, wherein applying the adaptive correlation filter to the imaging data comprises cross-correlating the adaptive correlation filter with the imaging data.

17. The method of claim 2, wherein applying the firing filter to the target anchor location comprises convolving the image of the target anchor location with the firing filter.

18. The method of claim 1, wherein the imaging data includes a set of PET images of the target region obtained over a plurality of patient platform positions during a PET pre-scan, the method further including, prior to generating the filtered image:
aligning one or more images from the set of PET images with at least one CT image using a planned anchor location and/or contour of the target region;
updating the adaptive correlation filter based on the aligned set of PET images; and
acquiring additional imaging data.

19. The method of claim 18, further including, after calculating the radiation fluence:
delivering the calculated radiation fluence to the target region while acquiring further additional PET imaging data;
updating the anchor location based on the further additional PET imaging data; and
updating the adaptive correlation filter based on the additional PET imaging data.

20. The method of claim 19, further including evaluating a confidence metric of the filtered image to determine whether the confidence metric exceeds a threshold confidence value.

21. The method of claim 1, further comprising:
delivering the calculated radiation fluence while acquiring additional PET imaging data at a position of a patient platform; and
updating the adaptive correlation filter based on the additional PET imaging data.

22. The method of claim 21, wherein the patient platform may be placed into a plurality of positions, and wherein the method includes repeating at least once:
moving the patient platform to a next position, such that the next position becomes the position at which the calculated radiation fluence is being delivered, and at which the adaptive correlation filter is being updated.

23. (canceled)

24. The method of claim 21, further comprising generating a graphical representation of the acquired PET imaging data.

25. The method of claim 21, wherein the calculated radiation fluence is derived from the acquired PET imaging data that has been filtered by the adaptive correlation filter.

26. The method of claim 1, further comprising:
delivering the calculated radiation fluence while acquiring additional PET imaging data during a patient platform shuttle pass; and
updating the adaptive correlation filter based on the additional PET imaging data.

27. The method of claim 26, wherein a plurality of couch shuttle passes is performed, and wherein steps of the method of claim 26 are performed for each pass of the plurality of couch shuttle passes.

28. The method of claim 1, wherein the target anchor location is the location of a first target anchor, the adaptive correlation filter is a first adaptive correlation filter, and
the filtered image is a first filtered image, and wherein the method further comprises determining a target region rotation or tilt using the location of the first target anchor, a location of a second target anchor and a location of a third target anchor, and
wherein calculating the radiation fluence to be delivered comprises applying the target region rotation or tilt to a planned radiation fluence.

29. The method of claim 28, wherein determining the target region rotation or tilt comprises:
generating a second filtered image by applying a second adaptive correlation filter to the imaging data;
determining the second target anchor location from the second filtered image;
generating a third filtered image by applying a third adaptive correlation filter to the imaging data;
determining the third target anchor location from the third filtered image;
defining a delivery orientation plane using the first, second and third target anchor locations; and
determining an angular rotation or tilt of the target region by comparing the delivery orientation plane with a planning orientation plane of the target region.

30. The method of claim 28, wherein the acquired imaging data is 3-D imaging data, and the first target anchor location, the second target anchor location, and the third target anchor location are defined using 3-D coordinates.

31. A radiation therapy system comprising:
a rotatable gantry;
a therapeutic radiation source mounted on the gantry;
one or more imaging sensors mounted on the gantry to acquire imaging data of a target region; and
a controller in communication with the gantry, the therapeutic radiation source, and the one or more imaging sensors, the controller configured to apply an adaptive correlation filter to the acquired imaging data to generate a cross-correlation image, determine a target anchor location from the cross-correlation image, and to calculate a radiation fluence to be delivered to the target region by convolving an image of the target anchor location with a firing filter.

32. The system of claim 31, wherein the firing filter is calculated based on previously-acquired imaging data of the target region.

33. The system of claim 31, wherein the controller is further configured to deliver the calculated radiation fluence to the target region with the therapeutic radiation source.

34. The system of claim 31, wherein the controller is further configured to update the adaptive correlation filter with additional imaging data.

35. The system of claim 34, wherein the controller is further configured to acquire the additional imaging data prior to updating the adaptive correlation filter.

36. The system of claim 35, wherein the additional imaging data is simulation imaging data.

37. The system of claim 31, wherein the acquired imaging data is obtained using a diagnostic positron emission tomography (PET) imaging system.

38. The system of claim 31, wherein the controller is further configured to:
determine a location of a planning contour using the acquired imaging data, and
wherein determining the target anchor location comprises locating the target anchor relative to the location of the planning contour.

39. The system of claim 38, wherein the planning contour is the boundary of a planned target volume (PTV), and the target anchor location is a location of a centroid of the PTV.

40. The system of claim 38, wherein the planning contour is the boundary of a planned target volume (PTV), and the target anchor location is a point on or within the boundary of the PTV.

41. The system of claim 40, wherein the planning contour is the boundary of an organ-at-risk (OAR), and the target anchor location is a point on or within the boundary of the OAR.

42. The system of claim 31, wherein the controller is further configured to:
deliver the calculated radiation fluence;
acquire additional imaging data;
update the target anchor location;
calculate a second radiation fluence using the updated target anchor location; and
deliver the second radiation fluence.

43. The system of claim 42, wherein:
the acquired imaging data is obtained using a diagnostic positron emission tomography (PET) imaging system; and
the acquired additional image data is obtained using a biology-guided radiotherapy (BgRT) PET imaging system.

44. The system of claim 31, wherein applying the adaptive correlation filter to the imaging data includes cross-correlating the adaptive correlation filter with the imaging data.

45. The system of claim 31, wherein the imaging data includes a set of PET images of the target region obtained over a plurality of patient platform positions during a PET pre-scan, the controller is further configured to:
align one or more images from the set of PET images with at least one CT image using a planned target anchor location and/or contour of the target region;
update the adaptive correlation filter based on the aligned set of PET images; and
acquire additional imaging data.

46. The system of claim 45, wherein the controller is further configured to, after calculating the radiation fluence:
deliver the calculated radiation fluence to the target region while acquiring additional PET imaging data;
update the target anchor location based on the further additional PET imaging data; and
update the adaptive correlation filter based on the additional PET imaging data.

47. The system of claim 31, wherein applying the adaptive correlation filter to the imaging data includes cross-correlating the adaptive correlation filter with the imaging data resulted in a filtered image, and wherein the controller is further configured to evaluate a confidence metric of the filtered image to determine whether the confidence metric exceeds a threshold confidence value.

48. The system of claim 31, wherein the controller is further configured to:
deliver the calculated radiation fluence while acquiring additional PET imaging data at a position of a patient platform; and
update the adaptive correlation filter based on the additional PET imaging data.

49. The system of claim 48, wherein the patient platform may be placed into a plurality of positions, and wherein the controller is further configured to repeat at least once:
move the patient platform to a next position, such that the next position becomes the position at which the calculated radiation fluence is being delivered, and at which the adaptive correlation filter is being updated.

50. The system of claim 49, further including a display, and wherein the controller is further configured to generate a signal corresponding to a graphical representation of the acquired PET imaging data and output the signal to the display.

51. The system of claim 50, wherein the calculated radiation fluence is derived from the acquired PET imaging data that has been filtered by the adaptive correlation filter.

52. The system of claim 31, wherein the target anchor location is the location of a first target anchor location, the adaptive correlation filter is a first adaptive correlation filter, and the cross-correlation image is a first cross-correlation image, and wherein the controller is configured to
determine a target region rotation or tilt using the location of the first target anchor, a location of a second target anchor and a location of a third target anchor, and
wherein calculating the radiation fluence to be delivered comprises applying the target region rotation or tilt to a planned radiation fluence.

53. The system of claim 52, wherein determining the target region rotation or tilt comprises:
generating a second cross-correlation image by applying a second adaptive correlation filter to the imaging data;
determining the second target anchor location from the second cross-correlation image;
generating a third cross-correlation image by applying a third adaptive correlation filter to the imaging data;
determining the third target anchor location from the third cross-correlation image;
defining a delivery orientation plane using the first, second and third target anchor locations; and
determining an angular rotation or tilt of the target region by comparing the delivery orientation plane with a planning orientation plane of the target region.

54. The system of claim 52, wherein the acquired imaging data is 3-D imaging data, and the first target anchor location, the second target anchor location, and the third target anchor location are defined using 3-D coordinates.

55-69. (canceled)

70. A method of calculating a radiation fluence for delivery, the method comprising:
defining a plurality of target anchors for a contour of a target region on a planning image, wherein the target anchors define a planning orientation volume;
generating an adaptive correlation filter corresponding to each of the plurality of target anchors and the target region contour;
acquiring imaging data of the target region during radiation delivery session;
applying the adaptive correlation filters to the acquired imaging data to generate a cross-correlation image corresponding to each of the plurality of target anchors;
determining a target anchor locations in each of the cross-correlation images;
defining a delivery orientation volume using the identified target anchor locations;
determining an angular rotation or tilt of the target region by comparing the delivery orientation volume with the planning orientation volume; and
calculating a delivery radiation fluence by applying the angular rotation or tilt to a planned radiation fluence map.

71. The method of claim 70, wherein the planning orientation volume is a planning orientation plane and the delivery orientation volume is a delivery orientation plane.

72. The method of claim 70, further comprising training each of the generated adaptive correlation filters with additional imaging data.

73. The method of claim 70, wherein the plurality of target anchors comprise one or more of distinctive features or landmarks relative to the contour of the target region.

74. The method of claim 73, wherein the distinctive features or landmarks comprise locations that are geometrically-derived from the contour of the target region.

75. The method of claim 73, wherein the distinctive features or landmarks comprise locations of anatomical structures.

76. The method of claim 70, wherein the plurality of target anchors comprise points on projections of the target region contour on orthogonal planes in a 3-D coordinate system.

77. The method of claim 70, further comprising segmenting the delivery radiation fluence into a set of radiotherapy machine instructions.

78. The method of claim 77, wherein the radiotherapy machine instructions comprise multi-leaf collimator configurations and therapeutic radiation source pulse characteristics.

79. The method of claim 77, further comprising delivering radiation beamlets according to the radiotherapy machine instructions.

80-95. (canceled)
